(19) United States
(12) Patent Application Publication
(10) Pub. No.: US 2024/0419246 A1
(43) Pub. Date: Dec. 19, 2024

(54) HUMAN AUGMENTATION PLATFORM USING CONTEXT, BIOSIGNALS, AND LANGUAGE MODELS

(71) Applicant: Cognixion Corporation, Santa Barbara, CA (US)

(72) Inventors: Christopher Jason Ullrich, Ventura, CA (US); Joseph Andreas Forsland, Santa Barbara, CA (US); Benjamin Cortens, Kentville (CA); Carl Hagmann, Syracuse, NY (US); Lucas Carl Steuber, Santa Barbara, CA (US)

(73) Assignee: Cognixion Corporation, Santa Barbara, CA (US)

(21) Appl. No.: 18/335,864

(22) Filed: Jun. 15, 2023

(51) Int. Cl. G06F 3/01; A61N 1/20; G06F 40/284; G10L 13/08

(52) U.S. Cl. CPC G06F 3/015; A61N 1/20; G06F 40/284; G10L 13/08

(57) ABSTRACT

There are disclosed herein systems and methods for human agency support and facilitation through an integrated use of context information, historical work product, biosensors, explicit user input and a generative AI or generalist agent. The system comprises input means, tokenization, a generative AI or generalist agent and an output stage capable of enacting agency on a user's behalf using output tokens from the language model.

### BACKGROUND

Human agency as a term of human psychology may refer to an individual's capacity to actively and independently make choices and to impose those choices on their surroundings. There are many situations in which people have a need and desire to make choices in interacting with their environment but are unable to do so without assistance. In this manner. such people find themselves impaired in their human agency to effect a change in their surroundings or communicate with those around them.

Advances in augmented and virtual reality, as well as the field of robotics and artificial intelligence (AI), offer a host of tools whereby a user unable to enact their agency to interact with the world around them unassisted may be supported in doing so. These systems may remain partially or fully inaccessible to users unable to speak, users with limited mobility, users with impaired perception of their surroundings, either sensory perception or social perception, and most of all, users inexperienced in interacting with augmented reality (AR), virtual reality (VR), and robotics.

Recent advances in Generative AI (GenAI) may allow those unfamiliar with coding to interact with AI and robotic assistants, as well as the people around them, using GenAI outputs. “Generative AI” or “GenAI” in this disclosure refers to a type of Artificial Intelligence (AI) capable of creating a wide variety of data, such as images, videos, audio, text, and 3D models. It does this by learning patterns from existing data, then using this knowledge to generate new and unique outputs. GenAI is capable of producing highly realistic and complex content that mimics human creativity, making it a valuable tool for many industries such as gaming, entertainment, and product design (found on https://generativeai.net, accessed May 24, 2023).

GenAI may include large language models (LLMs), Generative Pre-trained Transformer (GPT) including chatbots such as ChatGPT. OpenAI, text-to-image and other visual art creators such as Midjourney and Stable Diffusion, and even more comprehensive models such as the generalist agent Gato. However, conventional interaction with these entities involves formulating effective natural language queries, and this may not be possible for all users.

There is, therefore, a need for a system capable of creating effective GenAI prompts, based on information other than natural language provided by a user, in support of augmenting or facilitating the user's human agency.

### Brief Summary

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates a user agency and capability augmentation system 100 in accordance with one embodiment.

FIG. 2 illustrates a biosignals subsystem 200 in accordance with one embodiment.

FIG. 3 illustrates a context subsystem 300 in accordance with one embodiment.

FIG. 4 illustrates a routine 400 in accordance with one embodiment.

FIG. 5 illustrates a turn-taking capability augmentation system 500 in accordance with one embodiment.

FIG. 6 illustrates a user agency and capability augmentation system with output adequacy feedback 600 in accordance with one embodiment.

FIG. 7 illustrates a routine 700 in accordance with one embodiment.

FIG. 8 illustrates an exemplary tokenizer 800 in accordance with one embodiment.

FIG. 9A illustrates an isometric view of a BCI headset system 900 in accordance with one embodiment.

FIG. 9B illustrates a rear view of a BCI headset system 900 in accordance with one embodiment.

FIG. 9C and FIG. 9D illustrate exploded views of a BCI headset system 900 in accordance with one embodiment.

FIG. 10 illustrates a routine 1000 for improving indoor navigation for a user in accordance with one embodiment.

FIG. 11 illustrates a routine 1100 in accordance with one embodiment.

FIG. 12 illustrates a routine 1200 in accordance with one embodiment.

FIG. 13 illustrates a routine 1300 in accordance with one embodiment.

FIG. 14 illustrates a routine 1400 in accordance with one embodiment.

FIG. 15 illustrates a routine 1500 in accordance with one embodiment.

FIG. 16 illustrates a routine 1600 in accordance with one embodiment.

FIG. 17 illustrates a routine 1700 in accordance with one embodiment.

FIG. 18 illustrates a routine 1800 in accordance with one embodiment.

FIG. 19 illustrates a routine 1900 in accordance with one embodiment.

FIG. 20 illustrates a routine 2000 in accordance with one embodiment.

FIG. 21 illustrates a routine 2100 in accordance with one embodiment.

FIG. 22 illustrates a routine 2200 in accordance with one embodiment.

FIG. 23 illustrates a routine 2300 in accordance with one embodiment.

FIG. 24 illustrates a routine 2400 in accordance with one embodiment.

FIG. 25 illustrates a routine 2500 in accordance with one embodiment.

FIG. 26 illustrates a routine 2600 in accordance with one embodiment.

FIG. 27 illustrates a routine 2700 in accordance with one embodiment.

FIG. 28 illustrates a routine 2800 in accordance with one embodiment.

FIG. 29 illustrates a routine 2900 in accordance with one embodiment.

FIG. 30 illustrates a routine 3000 in accordance with one embodiment.

FIG. 31 illustrates a routine 3100 in accordance with one embodiment.

FIG. 32 illustrates a routine 3200 in accordance with one embodiment.

FIG. 33 illustrates a routine 3300 in accordance with one embodiment.

FIG. 34 illustrates a routine 3400 in accordance with one embodiment.

FIG. 35 illustrates a routine 3500 in accordance with one embodiment.

FIG. 36 illustrates a routine 3600 in accordance with one embodiment.

FIG. 37 illustrates a BCI+AR environment 3700 in accordance with one embodiment.

FIG. 38 illustrates an augmented reality device logic 3800 in accordance with one embodiment.

FIG. 39 illustrates a block diagram of nonverbal multi-input and feedback device 3900 in accordance with one embodiment.

FIG. 40 illustrates a block diagram of a single framework of a nonverbal multi-input and feedback device 4000 in accordance with one embodiment.

FIG. 41 illustrates a block diagram of nonverbal multi-input and feedback device 4100 in accordance with one embodiment.

FIG. 42 illustrates a logical diagram of a user wearing an augmented reality headset 4200 in accordance with one embodiment.

FIG. 43 a logical diagram of a user wearing an augmented reality headset 4300 in accordance with one embodiment.

FIG. 44 illustrates a diagram of a use case including a user wearing an augmented reality headset 4400 in accordance with one embodiment.

FIG. 45 illustrates a flow diagram 4500 in accordance with one embodiment.

FIG. 46 illustrates a flow diagram 4600 in accordance with one embodiment.

FIG. 47 illustrates a block diagram 4700 in accordance with one embodiment.

FIG. 48 illustrates a block diagram 4800 in accordance with one embodiment.

FIG. 49 illustrates a block diagram 4900 in accordance with one embodiment.

FIG. 50 illustrates a computing device 5000 in accordance with one embodiment.

FIG. 51 illustrates a cloud computing system 5100 in accordance with one embodiment.

FIG. 52 illustrates cloud computing functional abstraction layers 5200 in accordance with one embodiment.

### DETAILED DESCRIPTION

There are disclosed herein systems and methods for human agency support through an integrated use of context information from the user's environment, the user's historical data such as recorded usage of the system, a body of recorded historical work product, etc., biosensor signals indicating information about the user's physical state and bodily actions, and explicit user input. These are input to a prompt composer capable of taking in these inputs, generating a prompt for a generative AI or generalist agent.

The output of the generative AI or generalist agent may drive an output stage that is able to support and facilitate the human agency of the user based on the output. In this manner, the user may interact with the system with ease and rapidity, the system generating communications to or instructing the actions of supportive and interactive entities surrounding the user, such as other people, robotic aids, smart systems, etc.

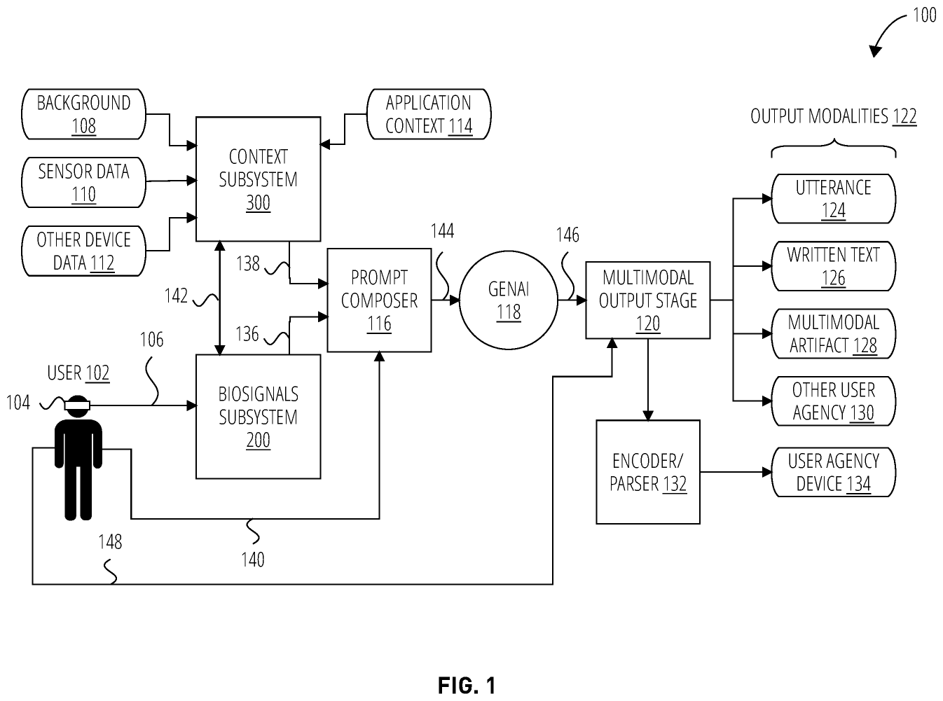

FIG. 1 illustrates a user agency and capability augmentation system 100 in accordance with one embodiment. The user agency and capability augmentation system 100 comprises a user 102, a wearable computing and biosignal sensing device 104, biosignals 106, background material 108, sensor data 110, other device data 112, application context 114 a prompt composer 116, a GenAI 118, a multimodal output stage 120, an encoder/parser 132, output modalities 122 such as an utterance 124, a written text 126, a multimodal artifact 128, an other user agency 130, and a non-language user agency device 134, a biosignals subsystem 200, and a context subsystem 300.

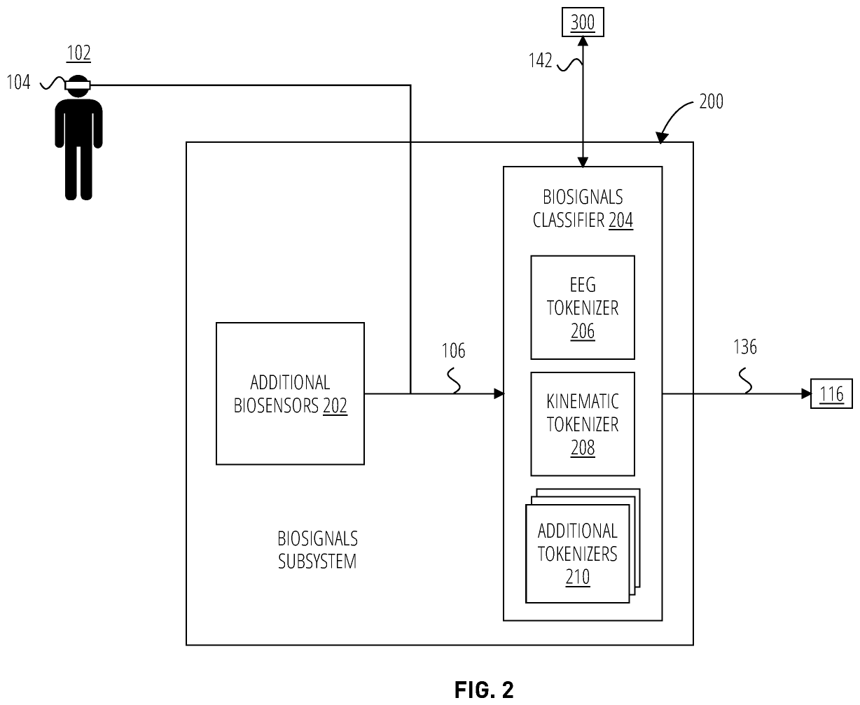

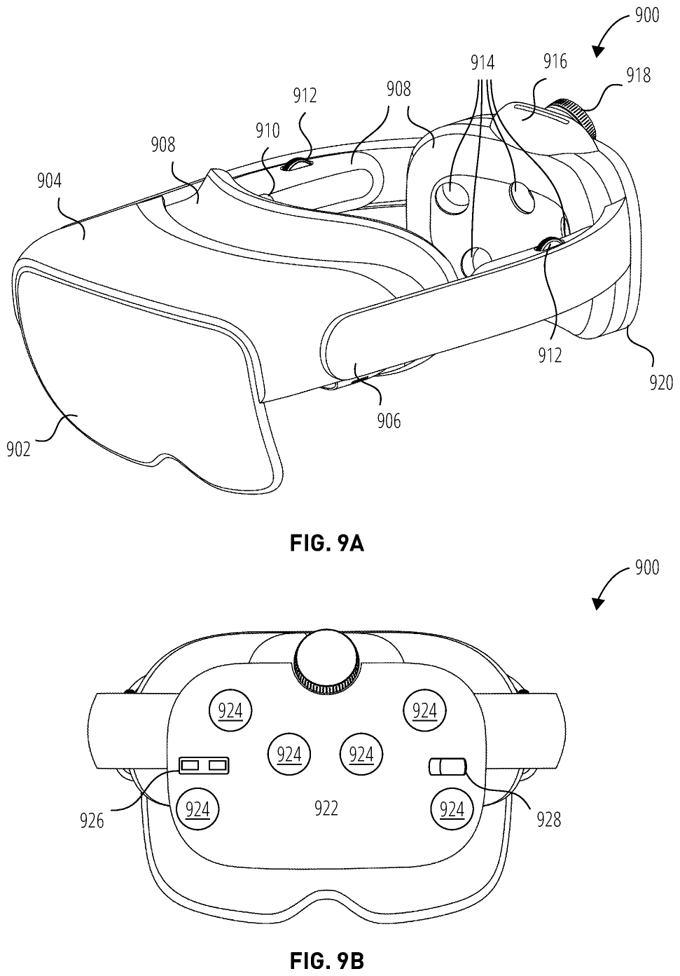

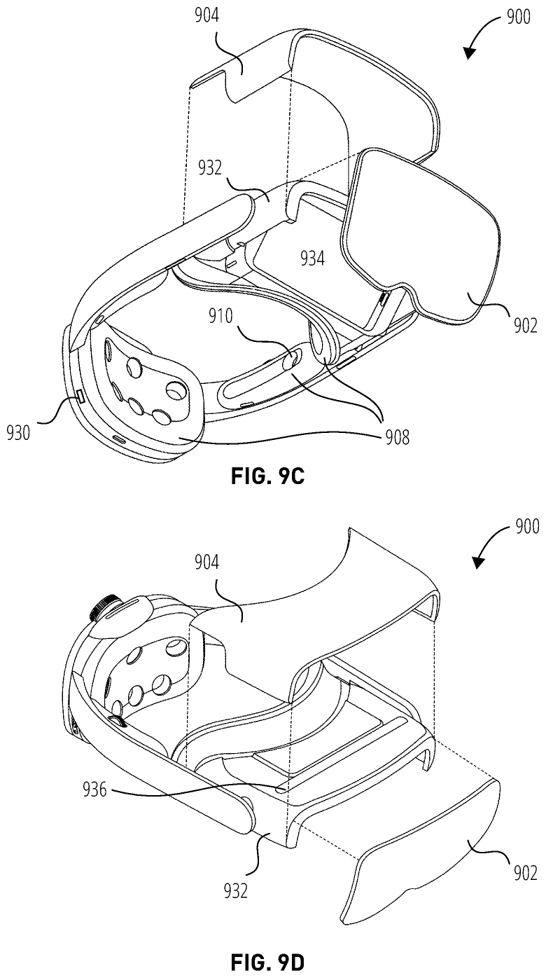

The user 102 in one embodiment may be equipped with and interact with a wearable computing and biosignal sensing device 104. The wearable computing and biosignal sensing device 104 may be a device such as the brain computer interface or BCI headset system 900 described in greater detail with respect to FIG. 9A through FIG. 9D. This embodiment may provide the user 102 with capability augmentation or agency support by utilizing biosignals 106, such as neurologically sensed signals and physically sensed signals, detected from the wearable computing and biosignal sensing device 104 and sent to a biosignals subsystem 200, in addition to data from biosignal sensors that may be part of the biosignals subsystem 200. The biosignals subsystem 200 may produce as its output a tokenized biosignals prompt 136. The action of the biosignals subsystem 200 is described in detail with respect to FIG. 2.

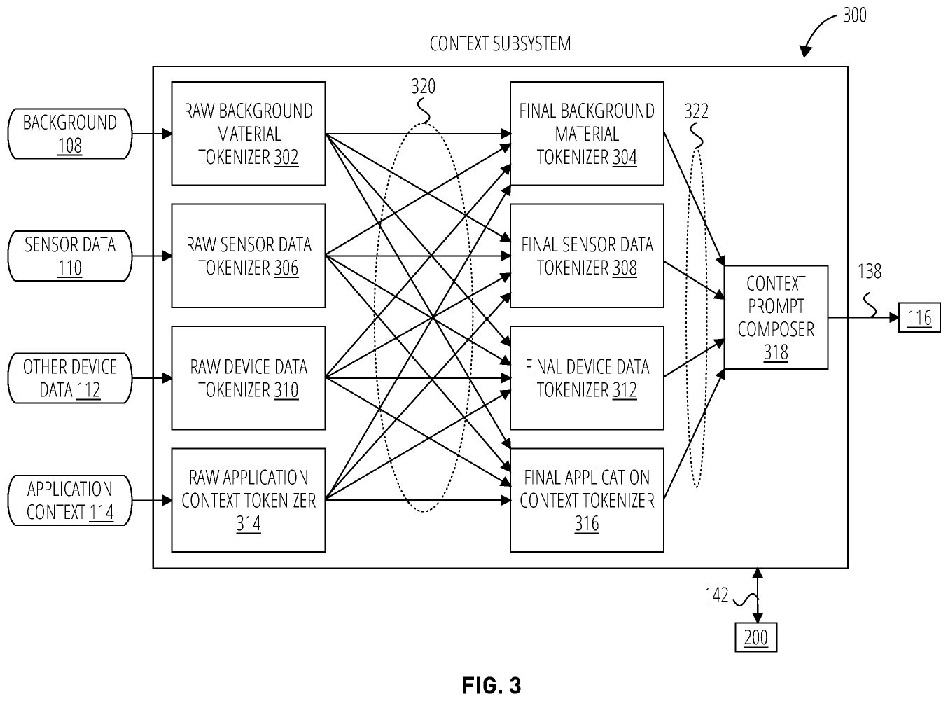

This embodiment may provide the user 102 with capability augmentation or agency support by utilizing inference of the user's environment, physical state, history, and current desired capabilities as a user context, to be gathered at a context subsystem 300, described in greater detail with respect to FIG. 3. This data may be provided as background material 108 on the user stored in a database or other storage structure, sensor data 110 and other device data 112 from a range of devices and on-device and off-device sensors, and application context 114 provided by applications, interfaces, or parameters configured to provide the capability augmentation sought by the user 102. The context subsystem 300 may produce as its output a tokenized context prompt 138.

In one embodiment, the biosignals subsystem 200 and the context subsystem 300 may be coupled or configured to allow shared data 142 to flow between them. For instance, some sensor data 110 or other device data 112 may contain biosignal information that may be useful to the biosignals subsystem 200. Or the biosignals subsystem 200 may capture sensor data 110 indicative of the user 102 context. These systems may communicate such data, in raw, structured, or tokenized forms, between themselves by means of wired or wireless communication. In one embodiment, these systems may operate as part of a device that is also configured and utilized to run other services.

This embodiment may finally provide the user 102 capability augmentation or agency support by utilizing direct user 102 input in the form of a user input prompt 140, such as mouse, keyboard, or biosignal-based selections, typed or spoken language, or other form of direct interaction the user 102 may have with a computational device that is part of or supports the user agency and capability augmentation system 100 disclosed. In one embodiment, the user 102 may provide an additional token sequence in one or more sensory modes, which may include a sequence of typed or spoken words, an image or sequence of images, and a sound or sequence of sounds. The biometric and optional multimodal prompt input from the user may be tokenized using equivalent techniques as for the context data.

The biosignals prompt 136, context prompt 138, and user input prompt 140 may be sent to a prompt composer 116. The prompt composer 116 may consume the data including the biosignals prompt 136, context prompt 138, and user input prompt 140 tokens, and may construct a single token, a set of tokens, or a series of conditional or unconditional commands suitable to use as a prompt 144 for a GenAI 118 such as a Large Language Model (LLM), a Generative Pre-trained Transformer (GPT) like GPT-4, or a generalist agent such as Gato. For example, a series such as “conditional on command A success, send command B, else send command C” may be built and sent all at once given a specific data precondition, rather than being built and sent separately.

The prompt composer 116 may also generate tokens that identify a requested or desired output modality (text vs. audio/visual vs. commands to a computer or robotic device, etc.) from among available output modalities 122 such as those illustrated. In one embodiment, the prompt composer 116 may further generate an embedding which may be provided separately to the GenAI 118 for use in an intermediate layer of the GenAI 118. In another embodiment, the prompt composer 116 may generate multiple tokenized sequences at once that constitute a series of conditional commands. In one exemplary use case, the user 102 submits a general navigational command to an autonomous robot or vehicle, such as “go to the top of the hill.” The prompt composer 116 may then interact with satellite and radar endpoints to construct specific motor commands, such as “Move forward 20 feet and turn left,” that navigate the robot or vehicle to the desired destination.

In one exemplary use case, the context subsystem 300 may generate a context prompt 138 token sequence corresponding to the plaintext, “The user has travelled to Los Angeles to visit a doctor specializing in rare diseases. The user is sitting in the doctor's office and preparing to discuss their disease. The user is looking at the doctor who has just asked the user for an update on their condition.” Such a context prompt 138 may be generated by utilizing sensors on a computing device worn or held by the user 102, such as a smart phone or the wearable computing and biosignal sensing device 104. Such sensors may include global positioning system (GPS) components, as well as microphones configured to feed audio to a speech to text (STT) device or module in order to identify the doctor and the questions. The biosignals subsystem 200 may generate a biosignals prompt 136 including a token sequence corresponding to the user selecting “speak” with a computing device to select this directive using an electroencephalography-based brain computer interface. The user input prompt 140 may include a token sequence corresponding to the plaintext, “The user has selected ‘summarize my recent disease experience’.” In this case, the prompt composer 116 may simply append these three token sequences into a single prompt 144 and may then pass it to the GenAI 118. In an alternate embodiment, the prompt composer 116 may replace the biosignals prompt 136 with a token sequence corresponding to the plaintext “Generate output in a format suitable for speech synthesis.”

In some embodiments, the prompt composer 116 may utilize a formal prompt composition language such as Microsoft Guidance. In such a case, the composition language may utilize one or more formal structures that facilitate deterministic prompt composition as a function of mixed modality inputs. For example, the prompt composer 116 may contain subroutines that process raw signal data and utilize this data to modify context prompt 138 and/or biosignals prompt 136 inputs in order to ensure specific types of GenAI 118 outputs.

A more intricate exemplary prompt from the prompt composer 116 to the GenAI 118, incorporating information detected from user 102 context and biosignals 106, may be as follows:

I am Sarah, a 60-year-old retired schoolteacher with advanced ALS. I enjoy the peaceful sounds of birds chirping. I just finished reading a mystery novel recommended by my friend Donna. As a literature enthusiast, I have a long history of discussing books with my friends and family I need help communicating and you are my assistant.

This is the current context:
Conversation history: Recent discussions about books with family and friends, including favorite authors, genres, and specific titles
Personal preferences: Fondness for mystery novels, historical fiction, and biographies; appreciation for strong character development and engaging plots
Language corpus and demographics: 60-year-old, retired school teacher, well-versed in literary terms and expressions
Mood: Content, relaxed, and eager to share her thoughts on the novel
Conversation Partner: Donna, my friend with shared interest in literature
Environmental audio: Sounds of birds chirping, rustling leaves, and distant neighborly conversations in the garden
Front-facing camera: Images of blooming flowers, lush greenery, and the mystery novel's cover
Location, motion, and positioning: At home, sitting in a comfortable garden chair
Reading preferences: Mystery novels, historical fiction, biographies, and classic literature
Educational background: Years of teaching experience in literature, familiarity with various literary periods and styles.

If I send an emoji, use it to topically or thematically improve prediction and alter tone. Use all contextual information and prior conversation history to modulate your responses. After each input, review the prior inputs and modify your subsequent predictions based on the context of the thread. Taking into account the current context, with spartan language, return a JSON string called ‘suggestions’ with three different and unique phrases without quotes. They should be complete sentences longer than two words. Do not include explanations. The phrases you respond with will be spoken by my speech generating device.

The GenAI 118 may take in the prompt 144 from the prompt composer 116 and use this to generate a multimodal output 146. The GenAI 118 may consist of a pre-trained machine learning model, such as GPT. The GenAI 118 may generate a multimodal output 146 in the form of a token sequence that may be converted back into plaintext, or which may be consumed by a user agency process directly as a token sequence. In an alternate embodiment, the output of the GenAI 118 further constitutes embeddings that may be decoded into multimodal or time-series signals capable of utilization by agency endpoints. Once determined, the output is digitally communicated to an agency endpoint capable of supporting the various output modalities 122.

In some embodiments, the GenAI 118 may generate two or more possible multimodal outputs 146 and the user 102 may be explicitly prompted at the multimodal output stage 120 to select between the choices. In the case of language generation, the user 102 may at the multimodal output stage 120 select between alternative utterances 124. In the case of robot control, the choices may consist of alternative paths that a robot could take in order to achieve a user-specified goal. In these embodiments, there may be an output mode selection signal 148 provided by the user 102 explicitly or indicated through biosignals 106, to the multimodal output stage 120. The output mode selection signal 148 may instruct a choice between the multimodal outputs 146 available from the GenAI 118 at the multimodal output stage 120. In one embodiment, the user 102 may further direct one or more of the alternatives to alternate endpoints supporting the various output modalities 122. For example, the user 102 may select one utterance 124 for audible presentation and a different one for transformation and/or translation to written text 126.

In an alternate configuration, the user agency and capability augmentation system 100 may contain multiple GenAIs 118, each of which is pre-trained on specific application, context, or agency domains. In this configuration, the context subsystem 300 may be responsible for selecting the appropriate GenAI 118 or GenAIs 118 for the current estimated user context. In some embodiments, mixture-of-experts models such as a generalist language model may be used for this.

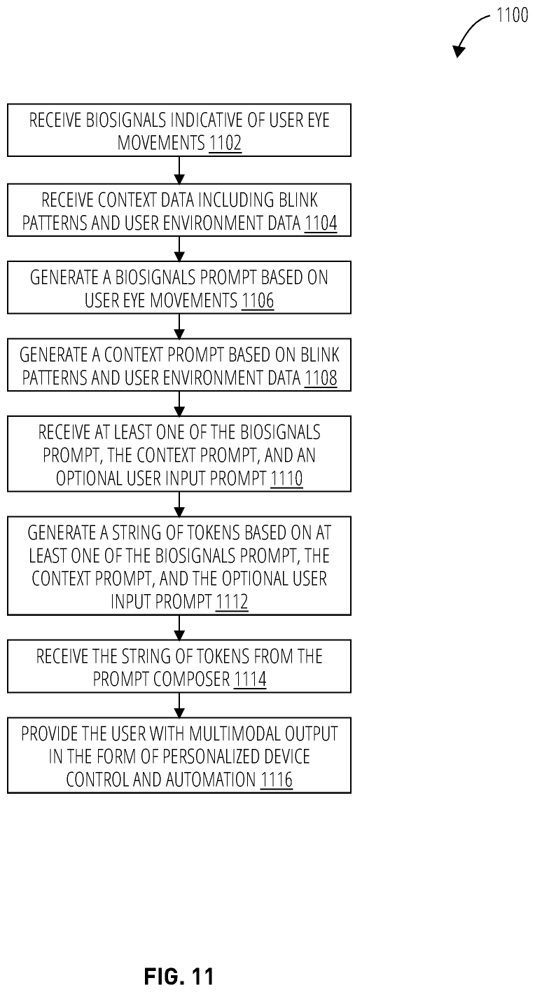

In some embodiments, models may be fine-tuned by the user 102. For example, the user 102 may provide a GenAI 118 LLM classifier model with exemplars of classes, either by speaking or writing them, and through few-shot learning, the model may improve accuracy. The multimodal outputs 146 may be made available to the user 102 through the agency endpoints supporting the various output modalities 122, and the user 102 may respond in a manner detectable through the user's biosignals 106, or directly through an additional user input prompt 140, and in this manner may also provide data through which the GenAI 118 may be refined. This is described more thoroughly with respect to FIG. 11.

The multimodal outputs 146 may be used to extend and support user 102 agency and augment user 102 capability into real and virtual endpoints. In one embodiment, the selected user agency process may be a speech synthesis system capable of synthesizing a token sequence or text string as a spoken language utterance 124 in the form of a digital audio signal. In another embodiment, the system's output may be constrained to a subset of domain-relevant utterances 124 for applications such as employment, industry, or medical care. This output constraint may be implemented using a domain specific token post-processing system or it may be implemented with an alternate GenAI that has been pre-trained on the target domain. In another embodiment, the endpoint may be a written text 126 composition interface associated with a communication application such as email, social media, chat, etc., or presented on the user's or their companions' mobile or wearable computing device. In a further embodiment, the output may be a multimodal artifact 128 such as a video with text, an audio file, etc. In another embodiment, the output may augment some other user agency 130, such as by providing haptic stimulation, or through dynamic alteration of a user's interface, access method, or complexity of interaction, to maximize utility in context.

In some embodiments, the multimodal outputs 146 may be additionally encoded using an encoder/parser 132 framework such as an autoencoder. In this system, the output of the encoder/parser 132 framework may be a sequence of control commands to control a non-language user agency device 134 or robotic system such as a powered wheelchair, prosthetic. powered exoskeleton, or other smart, robotic, or AI-powered device. In one embodiment, the prompt 144 from the prompt composer 116 may include either biosignals prompt 136 or user input prompt 140 tokens which represent the user's desired configuration, and the multimodal output includes detailed steps that a robotic controller may digest, once encoded by the encoder/parser 132. In this embodiment, the user 102 may express a desire to move from location A to location B, and the combination of the GenAI 118 and the robot controller may generate an optimal path as well as detailed control commands for individual actuators. In another embodiment, biosignals 106 may be used to infer a user's comfort with the condition of their surroundings, their context indicating that they are at home, and a prompt may be developed such that the GenAI 118 provides multimodal outputs 146 instructing a smart home system to adjust a thermostat, turn off music, raise light levels, or perform other tasks to improve user comfort. In a further embodiment, the GenAI 118 may generate a novel control program which is encoded by parsing or compiling it for the target robot control platform at the encoder/parser 132. The multimodal output 146 may through these methods be available as information or feedback to the user 102, through presentation via the wearable computing and biosignal sensing device 104 or other devices in the user's immediate surroundings. The multimodal output 146 may be stored and become part of the user's background material 108. The user 102 may respond to the multimodal output 146 in a manner detectable through biosignals 106, and thus a channel may be provided to train the GenAI 118 based on user 102 response to multimodal output 146.

In general, the user agency and capability augmentation system 100 may be viewed as a kind of application framework that uses the biosignals prompt 136, context prompt 138, and user input prompt 140 sequences to facilitate interaction with an application, much as a user 102 would use their finger to interact with a mobile phone application running on a mobile phone operating system. Unlike a touchscreen or mouse/keyboard interface, this system incorporates real time user inputs along with an articulated description of their physical context and historical context to facilitate extremely efficient interactions to enable user agency. FIG. 1 shows the pathways signals take from input, by sensing devices, stored data, or the user 102, to output in the form of text-to-speech utterances 124, written text 126, multimodal artifacts 128, other user agency 130 supportive outputs, and/or commands to a non-language user agency device 134. It will be well understood by one of skill in the art that not all components of the disclosed user agency and capability augmentation system 100 may be used in every application such a system may operate within or may not be used with equal weight. Some applications may make greater use of biosignals 106 than of context indicating the user history and surroundings. Some applications may necessitate operation completely independent from user input prompt 140 data. The disclosed user agency and capability augmentation system 100 may be used in support of such user applications as are described in the embodiments disclosed herein.

FIG. 2 illustrates a biosignals subsystem 200 in accordance with one embodiment. The biosignals subsystem 200 may comprise additional biosensors 202, a biosignals classifier 204, an electroencephalography or EEG Tokenizer 206, a kinematic tokenizer 208, and additional tokenizers 210, each of which may be suitable for one or more streams of biosignal data.

In addition to sensors which may be available on the wearable computing and biosignal sensing device 104 worn by the user 102, additional biosensors 202 may be incorporated into the biosignals subsystem 200. These may be of a mixture of physical sensors on or near the user's body that connect with network-connected and embedded data sources and models to generate a numerical representation of a biosignal estimate. An appropriate biosignal tokenizer may encode the biosignal estimate with associated data to generate at least one biosignal token sequence. In some embodiments, the mobile or wearable computing and biosignal sensing device 104 may include a set of sensory peripherals designed to capture user 102 biometrics. In this manner, the biosignals subsystem 200 may receive biosignals 106, which may include at least one of a neurologically sensed signal and a physically sensed signal.

Biosignals 106 may be tokenized through the use of a biosignals classifier 204. In some embodiments, these biometric sensors may include some combination of electroencephalography (EEG), electrocorticography (ECoG), electrocardiogram (ECG or EKG), electromyography (EMG), electrooculography (EOG), pulse, heart rate variability, blood sugar sensing, dermal conductivity, etc. These biometric data may be converted into a biosignal token sequence in the biosignals classifier 204, through operation of the EEG Tokenizer 206, kinematic tokenizer 208, or additional tokenizers 210, as appropriate.

It is common practice for biosignal raw signal data to be analyzed in real time using a classification system. For EEG signals, a possible choice for an EEG Tokenizer 206 may be canonical correlation analysis (CCA), which ingests multi-channel time series EEG data and outputs a sequence of classifications corresponding to stimuli that the user may be exposed to. However, one skilled in the art will recognize that many other signal classifiers may be chosen that may be better suited to specific stimuli or user contexts. These may include but are not limited to independent component analysis (ICA), xCCA (CCA variants), power spectral density (PSD) thresholding, and machine learning. One skilled in the art will recognize that there are many possible classification techniques. In one example, these signals may consist of steady state visually evoked potentials (SSVEP) which occur in response to specific visual stimuli. In other possible embodiments, the classification may consist of a binary true/false sequence corresponding to a P300 or other similar neural characteristic. In some embodiments, there will be a user or stimuli specific calibrated signal used for the analysis. In other embodiments, a generic reference may be chosen. In yet other possible embodiments, the classes may consist of discrete event related potential (ERP) responses. It may be clear to one of ordinary skill in the art that other biosignals including EOG, EMG, and EKG, may be similarly classified and converted into symbol sequences. In other embodiments, the signal data may be directly tokenized using discretization and a codebook. The resulting tokens may be used as part of the biosignals prompt 136.

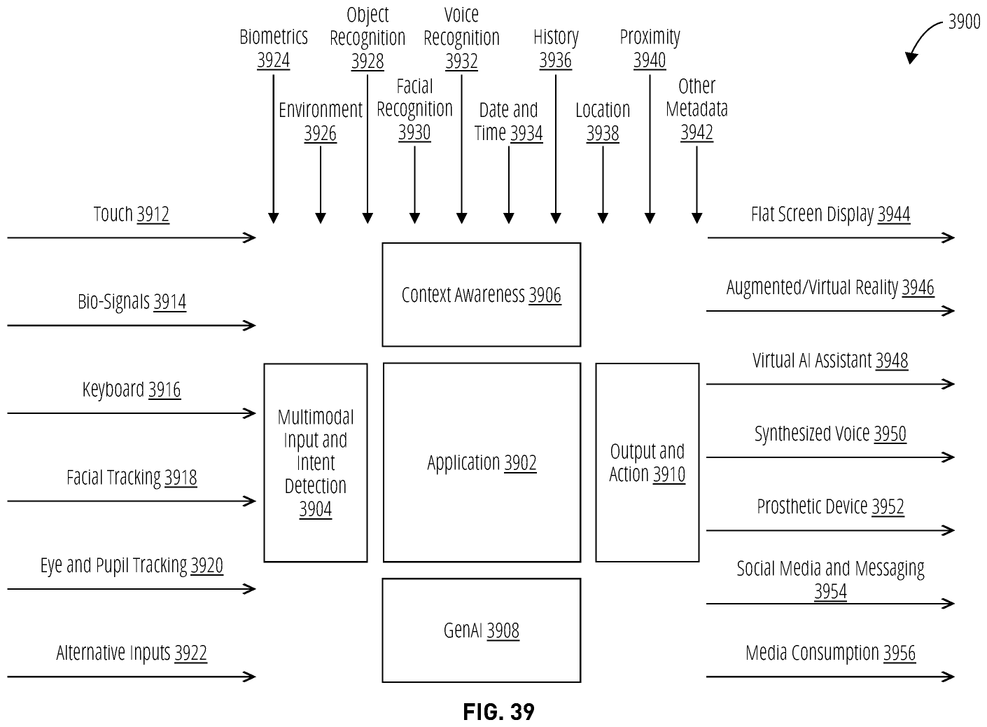

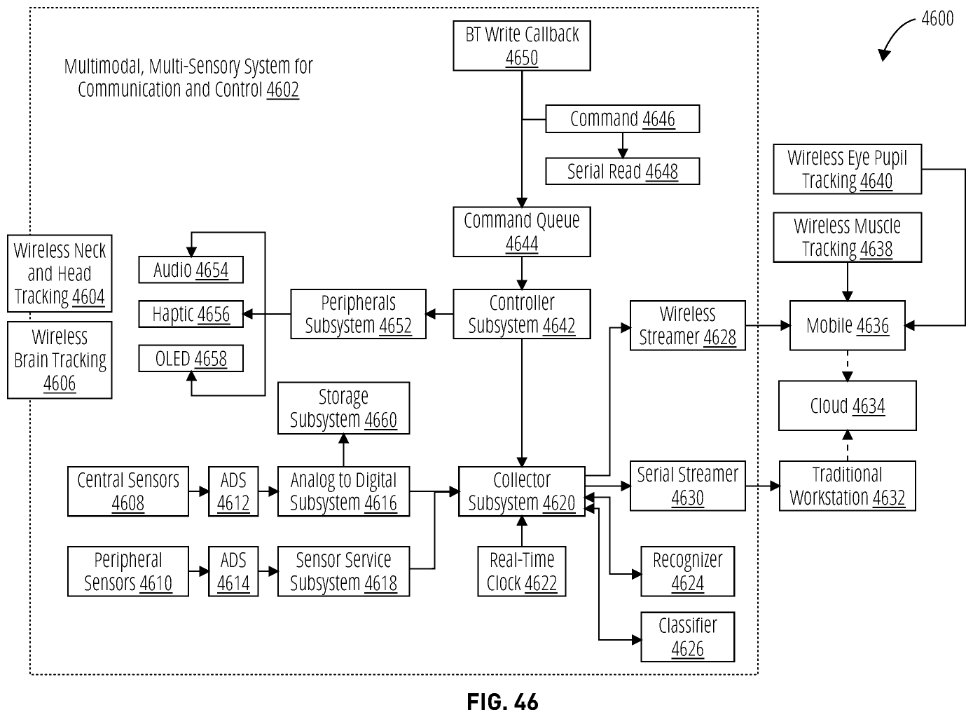

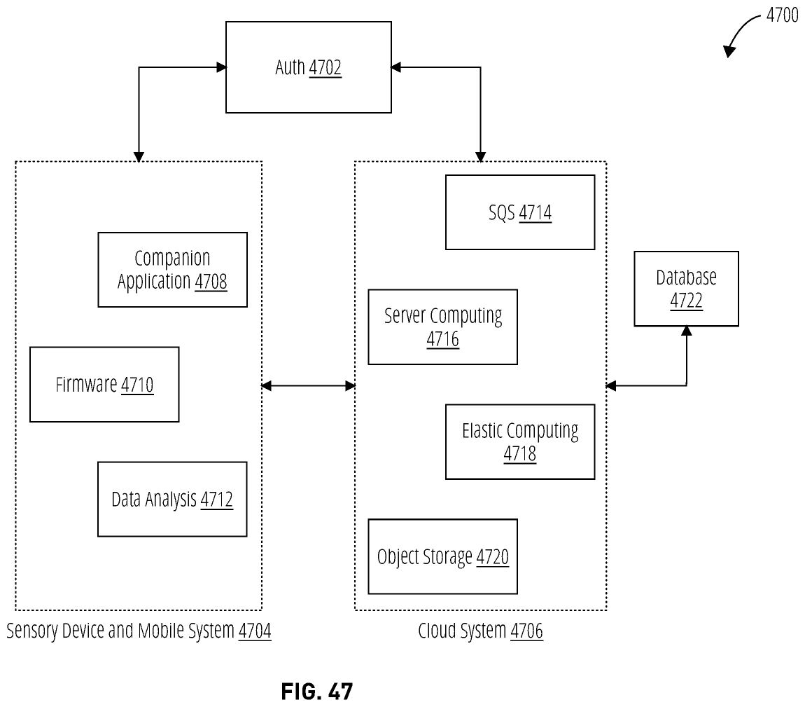

The kinematic tokenizer 208 may receive biosignals 106 indicative of user 102 motion, or motion of some part of a user's body, such as gaze detection based on the orientation and dilation of a user's pupils, through eye and pupil tracking discussed with reference to FIG. 39 or FIG. 46. Such kinematic biosignals 106 may be tokenized through the operation of the kinematic tokenizer 208 for inclusion in the biosignals prompt 136. Additional tokenizers 210 may operate similarly upon other types of biosignals 106. In one possible embodiment, the kinematic tokenizer 208 may utilize a codebook that maps state-space values (position/orientation, velocity/angular velocity) into codes which form the sequence of codes. In other embodiments, a model-based tokenizer may be used to convert motion data into discrete code sequences.

The final output from the biosignals subsystem 200 may be a sequence of text tokens containing a combination of the token sequences generated from the biosignals 106, in the form of the biosignals prompt 136. The biosignals subsystem 200 may also have a connection with the context subsystem 300 in advance of any prompt composition. This shared data 142 connection may bidirectionally inform each of the subsystems to enable more precise, or more optimal token generation.

FIG. 3 illustrates a context subsystem 300 in accordance with one embodiment. The context subsystem 300 may comprise a raw background material tokenizer 302, a final background material tokenizer 304, a raw sensor data tokenizer 306, a final sensor data tokenizer 308, a raw device data tokenizer 310, a final device data tokenizer 312, a raw application context tokenizer 314, a final application context tokenizer 316, and a context prompt composer 318.

Broadly speaking, the user's context consists of prompts generated from a variety of different data sources, including background material 108 that provides information about the user's previous history, sensor data 110 and other device data 112 captured on or around the user 102, and application context 114. i.e., information about the current task or interaction the user 102 may be engaged in.

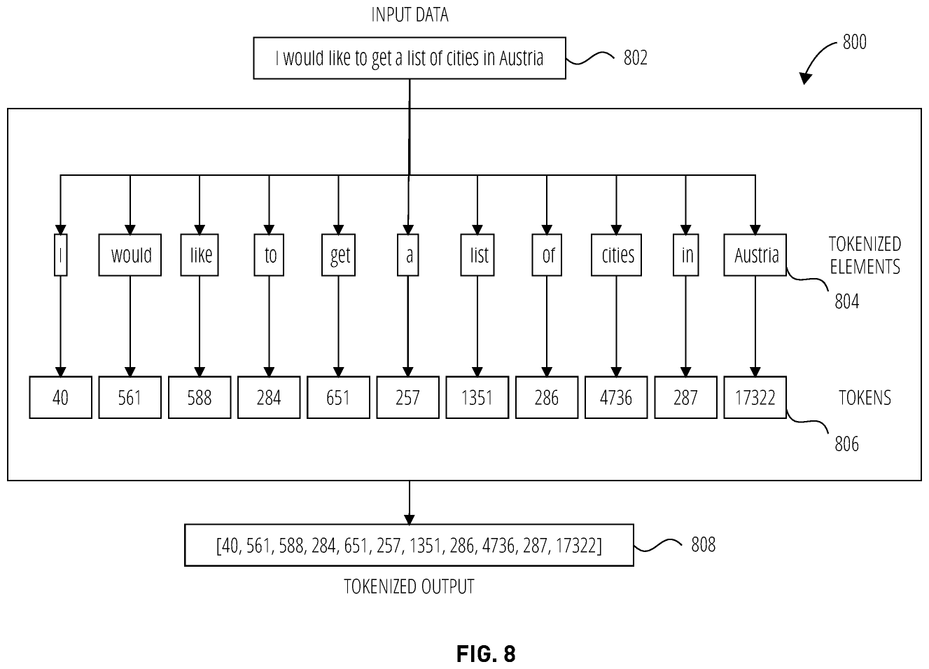

Background material 108 may be plain text, data from a structured database or cloud data storage (structured or unstructured), or any mixture of these data types. In one embodiment background material 108 may include textual descriptions of activities that the user 102 has performed or requested in a similar context and their prior outcomes, if relevant. In one embodiment, background material 108 may include general information about the user 102, about topics relevant to the user's current environment, the user's conversational histories, a body of written or other work produced by the user 102, or notes or other material related to the user's situation which is of a contextual or historical nature. In some embodiments, the background material 108 may first be converted into a plain text stream and then tokenized using a plaintext tokenizer. This is illustrated in greater detail with respect to FIG. 8.

Sensor data 110 may include microphone output indicative of sound in the user's environment, temperature, air pressure, and humidity data from climactic sensors, etc., output from motion sensors, and a number of other sensing devices readily available and pertinent to the user's surroundings and desired application of the user agency and capability augmentation system 100. Other device data 112 may include camera output, either still or video, indicating visual data available from the user's surrounding environment, location information from a global positioning system device, date and time data, information available via a network based on the user's location, and data from a number of other devices readily available and of use in the desired application of the user agency and capability augmentation system 100. Scene analysis may be used in conjunction with object recognition to identify objects and people present in the user's environment, which may then be tokenized. The context subsystem may also include a mixture of physical sensors such as microphones and cameras that connect with network-connected and embedded data sources and models to generate a numerical representation of a real-time context estimate.

In some instances, the user 102 may interact with an application on a computing device, and this interaction may be supported and expanded through the integration of a user agency and capability augmentation system 100. In these instances, explicit specification of the application may greatly enhance the context subsystem 300 knowledge of the user 102 context and may facilitate a more optimal context token set. Application context 114 data may in such a case be made available to the user agency and capability augmentation system 100, and data from the application context 114 data source may be tokenized as part of the operation of the context subsystem 300, for inclusion in the context prompt 138. Application context 114 data may include data about the current application (e.g., web browser, social media, media viewer, etc.) along with the user's interactions associated with the application, such as a user's interaction with a form for an online food order, data from a weather application the user is currently viewing, etc.

For each data source, a raw data tokenizer may generate a set of preliminary tokens 320. These preliminary tokens 320 may be passed to final tokenizers for all of the data sources to be consumed as input for the final tokenizers for each data source. Each data source final tokenizer may refine its output based on the preliminary tokens 320 provided by other data sources. This may be particularly important for background material 108. For example, the context used by the final background material tokenizer 304 to determine which background material 108 elements are likely to be relevant may be the prompt generated by the raw data source tokenizers. For example, camera data and microphone data may indicate the presence and identity of another person within the user's immediate surroundings. Background material 108 may include emails, text messages, audio recordings, or other records of exchanges between this person and the user, which the final background material tokenizer 304 may then include and tokenize as of particular interest to the user's present context.

The context subsystem 300 may send the final tokens output from the final tokenizers for each data source to a context prompt composer 318. The context prompt composer 318 may use these final tokens 322, in whole or in part, to generate a context prompt 138, which may be the final output from the context subsystem 300. The context prompt 138 may be a sequence of text tokens containing the combination of the background, audio/video, and other final tokens 322 from the final background material tokenizer 304, final sensor data tokenizer 308, final device data tokenizer 312, and final application context tokenizer 316. In the simplest embodiment, the context prompt composer 318 concatenates all the final tokens 322. In other possible embodiments, the context prompt composer 318 creates as its context prompt 138 a structured report that includes additional tokens to assist the GenAI in parsing the various final tokens 322 or prompts.

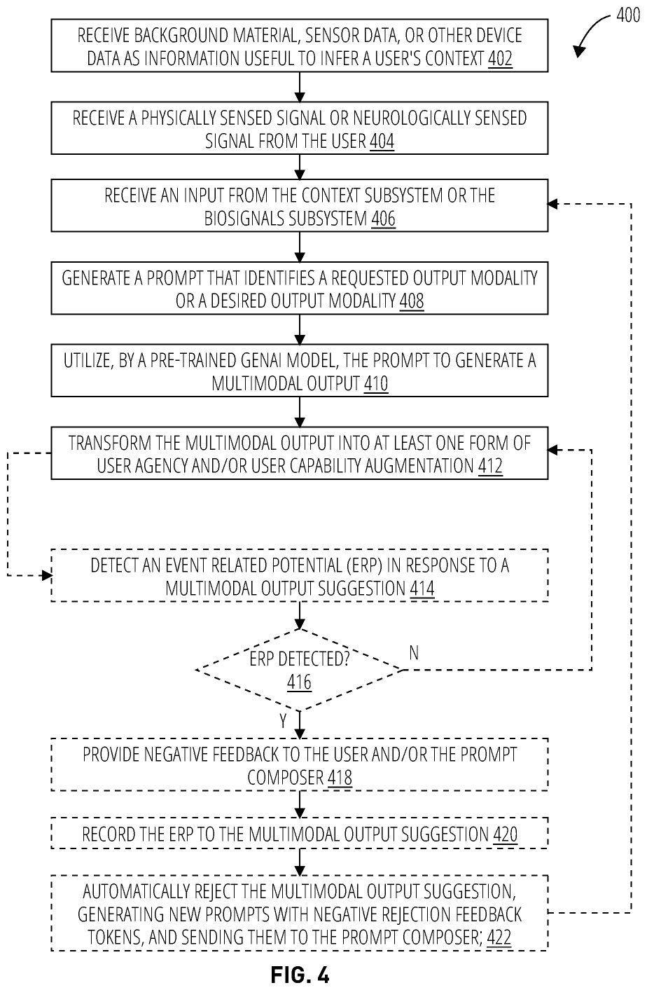

FIG. 4 illustrates an example routine 400 for implementation using a human augmentation platform using context, biosignals, and Generative AI. Such a routine 400 may be performed through the operation of a user agency and capability augmentation system 100 such as that illustrated and described with respect to FIG. 1. Although the example routine 400 depicts a particular sequence of operations, the sequence may be altered without departing from the scope of the present disclosure. For example, some of the operations depicted may be performed in parallel or in a different sequence that does not materially affect the function of the routine 400. In other examples, different components of an example device or system that implements the routine 400 may perform functions at substantially the same time or in a specific sequence.

According to some examples, the method includes receiving, by a context subsystem, at least one of background material, sensor data, and other device data as information useful to infer a user's context at block 402. For example, the context subsystem 300 illustrated in FIG. 3 may receive background material, sensor data, or other device data as information useful to infer a user's context. In one embodiment, the at least one of the sensor data and the other device data may be received by the context subsystem from at least one of a camera and a microphone array. In one embodiment, the context subsystem may also receive application context from applications installed on computing or smart devices in communication with the context subsystem.

According to some examples, the method includes receiving, by a biosignals subsystem, at least one of a physically sensed signal and a neurologically sensed signal from the user at block 404. For example, the biosignals subsystem 200 illustrated in FIG. 2 may receive a physically sensed signal or neurologically sensed signal from the user as the biosignals 106 introduced in FIG. 1. In one embodiment, the biosignals subsystem may receive biosignals data from biometric sensors for at least one of electroencephalography (EEG), electrocorticography (ECoG), electrocardiogram (ECG or EKG), electromyography (EMG), electrooculography (EOG), pulse determination, heart rate variability determination, blood sugar sensing, and dermal conductivity determination.

According to some examples, the method includes receiving, by a prompt composer, an input from at least one of the context subsystem and the biosignals subsystems; at block 406. For example, the prompt composer 116 illustrated in FIG. 1 may receive an input from the context subsystem or the biosignals subsystem. In one embodiment, the prompt composer may additionally receive a user input prompt. In this manner, a user may directly and explicitly provide instruction to the prompt composer.

According to some examples, the method includes generating, by the prompt composer, a prompt that identifies at least one of a requested output modality and a desired output modality at block 408. For example, the prompt composer 116 illustrated in FIG. 1 may generate a prompt that identifies a requested output modality or a desired output modality. In one embodiment, the prompt composer may generate at least one of a single token, a string of tokens, a series of conditional or unconditional commands suitable to prompt the GenAI model, tokens that identify at least one of the requested output modality and the desired output modality, an embedding to be provided separately to the GenAI model for use in an intermediate layer of the GenAI model, and multiple tokenized sequences at once that constitute a series of conditional commands.

According to some examples, the method includes utilizing, by a pre-trained Generative Artificial Intelligence (GenAI) model, the prompt to generate a multimodal output at block 410. For example, the GenAI 118 illustrated in FIG. 1 may utilize, by a pre-trained GenAI model, the prompt to generate a multimodal output. In one embodiment, the GenAI model may be at least one of large language models (LLMs), Generative Pre-trained Transformer (GPT) models, text-to-image creators, visual art creators, and generalist agent models.

According to some examples, the method includes transforming, by an output stage, the multimodal output into at least one form of user agency, user capability augmentation, and combinations thereof at block 412. For example, the multimodal output stage 120 illustrated in FIG. 1 may transform the multimodal output into at least one form of user agency and/or user capability augmentation. In one embodiment, the at least one form of the user agency includes neural stimulation to the user with Transcranial Direct Current Stimulation (tDCS), direct brain stimulation (DBS), or other known neural stimulation techniques. In one embodiment, the multimodal output may be in the form of at least one of text-to-speech utterances, written text, multimodal artifacts, other user agency supportive outputs, and commands to a non-language user agency device. In one embodiment, the output stage may receive an output mode selection signal from the user. The output mode selection signal may be a direct selection or may be generated from the user's biosignals. The output mode selection signal may instruct the output stage of a choice between multimodal outputs or may direct one or more alternative multimodal outputs to alternate endpoints. In one embodiment, an encoder/parser framework, such as the encoder/parser 122 of FIG. 1, may encode the multimodal output to provide control commands to control at least one of a non-language user agency device, a robot system, and smart AI-powered devices.

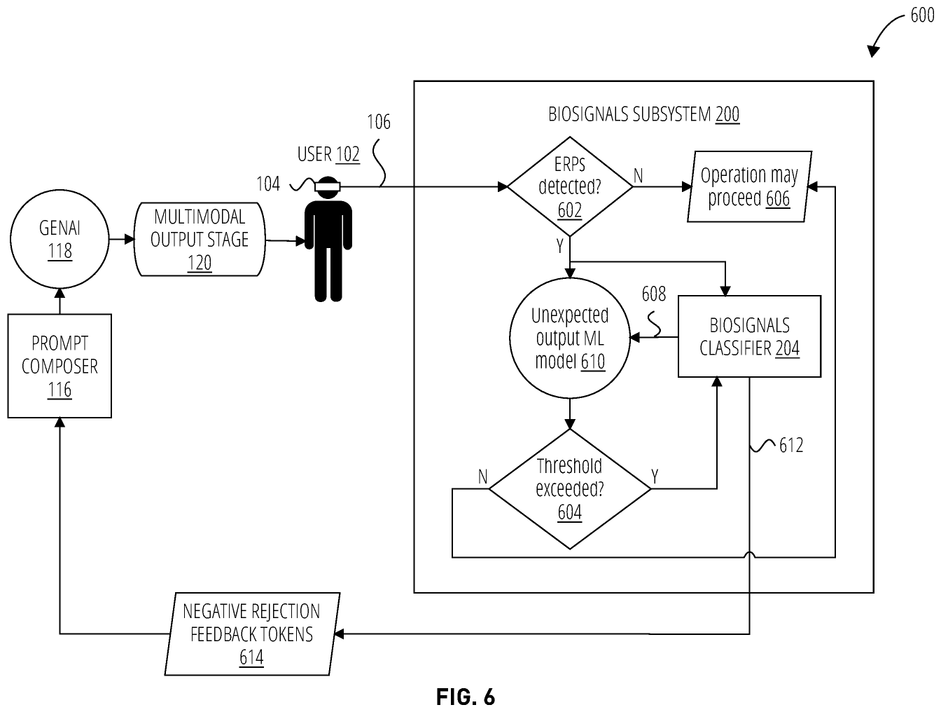

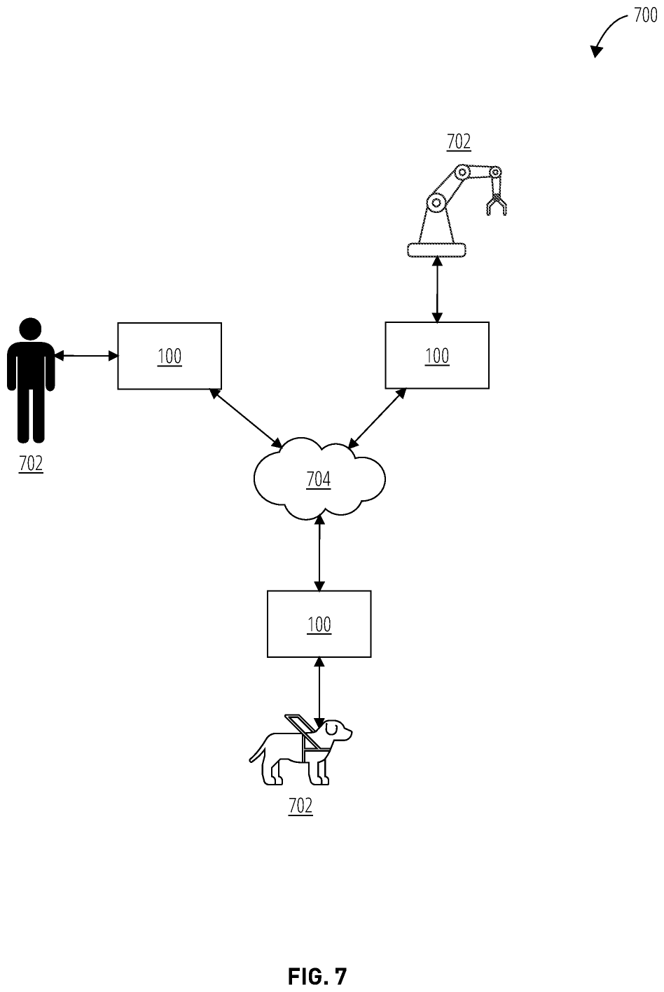

According to some examples, the method includes detecting, using an output adequacy feedback system, an event related potential (ERP) which may be an error-related negativity in response to a multimodal output suggestion at block 414. For example, the user agency and capability augmentation system with output adequacy feedback 600 illustrated in FIG. 6 may support detection of an ERP in response to a multimodal output suggestion. If an ERP is not detected at decision block 416, the method may include allowing the multimodal output suggestion to proceed.

According to some examples, the method includes, if an ERP is detected at decision block 416, providing negative feedback to at least one of the user and the prompt composer at block 418. The prompt composer may provide the negative feedback to the GenAI model.

According to some examples, the method includes, if an ERP is detected at decision block 416, recording the ERP to the multimodal output suggestion at block 420.

According to some examples, the method includes, if an ERP is detected at decision block 416, automatically rejecting the multimodal output suggestion, generating new prompts with negative rejection feedback tokens, and sending the negative rejection feedback tokens to the prompt composer at block 422.

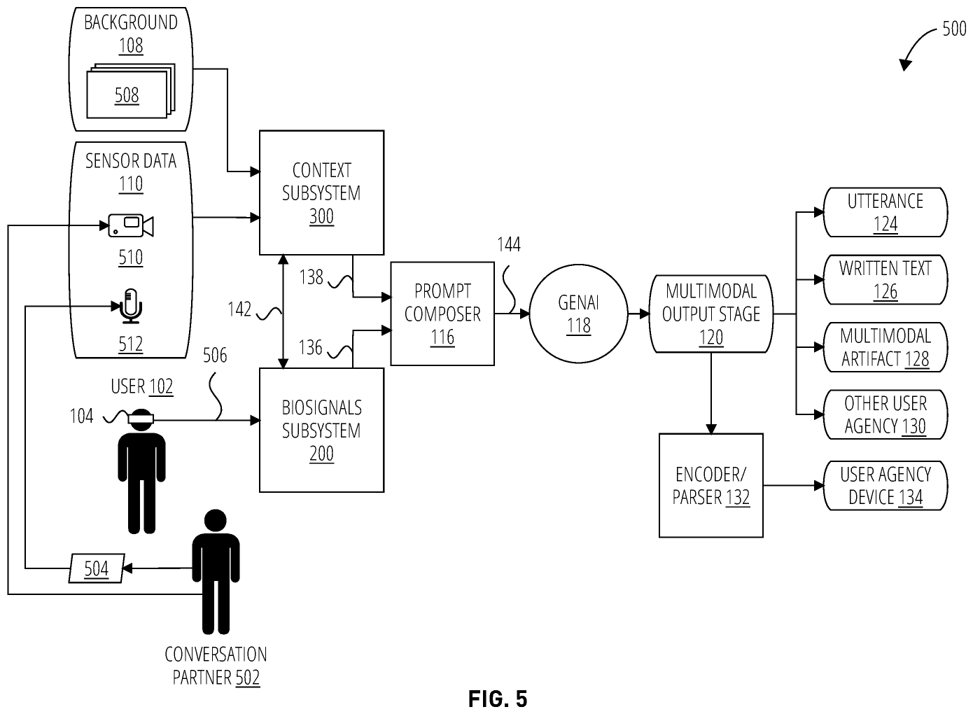

FIG. 5 illustrates a turn-taking capability augmentation system 500 in accordance with one embodiment. In the turn-taking capability augmentation system 500, the biosignals subsystem 200, context subsystem 300, prompt composer 116, and GenAI 118 may work together to detect when it is time for the user 102 to respond to a conversation partner 502 contributing audible speech 504. The turn-taking capability augmentation system 500 may comprise all components of the user agency and capability augmentation system 100, though certain elements are shown in additional detail or are simplified herein for ease of illustration and description.

The biosignals subsystem 200 may utilize brain sensing to capture and tokenize EEG or similar biosignals 506 indicating that the user 102 has detected or is anticipating a question or declination in speech which they are expected to respond to. The tokenized EEG or similar biosignals 506 may be used as the biosignals prompt 136 and may include anticipatory speech response EEG tokens. Microphone data 512 may record speech 504 from the conversation partner 502 for use in determining the appropriate response. At an experimentally determined threshold level of brain sensing anticipation and microphone data 512 silence, the conversation partner 502 speech 504 may be converted to text and tokenized by the context subsystem 300, along with conversation history and user knowledge of a topic 508. Camera data 510 showing the motion, stance, lip movements, etc., of the conversation partner 502 may also be tokenized by the context subsystem 300. This tokenized data may be used to generate the context prompt 138.

The biosignals prompt 136 and context prompt 138 may be combined by the prompt composer 116 as an automatic response to receiving the conversation partner 502 input. The prompt composer 116 may send a resulting prompt 144 to the GenAI 118. The GenAI 118 may take this input and generate multimodal outputs that may be used to produce responses that are tonally, semantically, modally, and temporally appropriate to the context provided, including but not limited to the new anticipatory brain sensing data, the speech to text from the conversation partner 502 microphone data 512, and the rest of the conversation history and user knowledge of a topic 508. In at least one embodiment, the user 102 may further provide input or direction to the turn-taking capability augmentation system 500 to select from among possible responses generated by the GenAI 118.

FIG. 6 illustrates a user agency and capability augmentation system with output adequacy feedback 600 in accordance with one embodiment. The biosignals subsystem 200 of the user agency and capability augmentation system with output adequacy feedback 600 may be configured to detect whether or not the multimodal output of a GenAI 118 may be automatically rejected based on a user's biosignals 106 in response to the output at the multimodal output stage 120. The user agency and capability augmentation system with output adequacy feedback 600 may comprise all components of the user agency and capability augmentation system 100, though certain elements are shown in additional detail or are simplified herein for case of illustration and description.

In one embodiment, after the GenAI 118 has generated a suggested item in the form of multimodal output, that multimodal output may be available to the perception of the user 102 through various endpoint devices, as previously described. Biosensors configured in the wearable computing and biosignal sensing device 104 or biosignals subsystem 200 may detect biosignals 106 indicating user 102 surprised or negative response, potentially indicating an unexpected or undesired multimodal output. Event related potentials (ERPs) are well known to those of skill in the art as indicating user surprise when presented with unexpected or erroneous stimuli. If no ERPs are detected in the biosignals 106 at decision block 602, operation may proceed 606 as usual.

If the user agency and capability augmentation system with output adequacy feedback 600 detects error/surprise in the form of an ERP at decision block 602, the user's response and actions in response to the multimodal output stage 120 output suggestion may be recorded, whether the multimodal output is ultimately accepted or rejected. The user 102 response itself, the strength of the response, and the number of sensors agreeing with the response, may be used in combination with the input tokens to the system (from the original prompt 608 for which the GenAI 118 produced the undesired multimodal output) to feed into an unexpected output machine learning model 610. This model may use supervised learning to determine what combination of error/surprise response+prompt token may be relied upon to predict when a user will reject or accept a suggestion. If the likelihood of suggestion rejection is too low (below an experimentally determined threshold or a user configured threshold), operation may proceed 606 as usual.

If the likelihood of suggestion rejection is sufficient to exceed the experimentally determined threshold or user configured threshold at decision block 604, the system may automatically reject the suggestion and generate a new prompt 612 including negative rejection feedback tokens 614. The automatic rejection feedback in the form of the new prompt 612 with negative rejection feedback tokens 614 may then be passed back into the prompt composer 116 to provide negative feedback to the GenAI 118. Feedback to the GenAI 118 may include the current context state (e.g. user heart rate, location, conversation partner, history, etc.) as well as the negative ERP.

For example, the GenAI 118 may generate an utterance that is positive in tone. However, the user 102 may be expecting a message with a negative tone. This incongruity may be detected in the user's biosignals 106. Sensing in the user agency and capability augmentation system with output adequacy feedback 600 may include a wearable computing and biosignal sensing device 104, such as a BCI headset system 900, which may be capable of eye and pupil tracking, smart device sensors, third-party sensing integrations, etc. These sensors may be capable of detecting EEG signals, EKG signals, heart rate, gaze direction, and facial expression. Such biosignals 106 as detected herein may show an elevation in heart rate, a widening of the user's eyes, a user's facial expression indicative of puzzlement or displeasure, etc. The biosignals subsystem 200 may then operate to generate a new prompt 612 that includes a rejection of the statements generated by the GenAI 118 in response to the original prompt 608. In some embodiments, the sensed user response information may be collected and used to refine the model after some period of time.

In the above embodiments, it may be understood by one skilled in the art that a record of inputs and responses may be used to retrain and enhance the performance of any of the system components. For example, a record of natural language outputs from the GenAI 118 may be scored based on some external measure and this data may then be used to retrain or fine-tune the GenAI 118.

All of the disclosed embodiments may provide for some type of feedback to a user 102 or another entity. One of ordinary skill in the art will readily apprehend that this feedback may be in the form of a sensory stimuli such as visual, auditory or haptic feedback. However, it may also be clear that this feedback may be transmitted over a network to a server which may be remote from the user 102. This remote device may further transmit the output from the system and/or it may transform the output into some other type of feedback which may then be communicated back to the user 102 and rendered as visual, auditory or haptic stimuli.

Some or all of the elements of the processing steps of the system may be local or remote to the user 102. In some embodiments, processing may be both local and remote while in others, key steps in the processing may leverage remote compute resources. In some embodiments, these remote resources may be edge compute while in others they may be cloud compute.

In some of the embodiments, the user 102 may explicitly select or direct components of the system. For example, the user 102 may be able to choose between GenAIs 118 that have been trained on a different corpus or training set if they prefer to have a specific type of interaction. In one example, the user 102 may select between a GenAI 118 trained on clinical background data or a GenAI 118 trained on legal background data. These models may provide distinct output tokens that are potentially more appropriate for a specific user-intended task or context.

### Simultaneous Users

In some embodiments more than one user 102 may be interacting simultaneously with a common artifact, environment, or in a social scenario. In these embodiments, each simultaneous user 702 may interact with an instance of one or more of the user agency and capability augmentation system 100 embodiments described herein.

Further, when multiple such user agency and capability augmentation systems 100 are present, they may establish direct, digital communication with each other via a local area or mesh network 704 to enable direct context transmission and exchange of GenAI 118 outputs.

In some instances, one or more of the simultaneous users 102 may be a robot or other autonomous agent. In yet other instances, one or more of the users 102 may be an assistive animal such as a sight impairment support dog.

FIG. 8 illustrates an exemplary tokenizer 800 in accordance with one embodiment. Input data 802 may be provided to the exemplary tokenizer 800 in the form of a text string typed by a user. In one embodiment, the text string may be generated by performing voice-to-text conversion on an audio stream. The exemplary tokenizer 800 may detect tokenizable elements 804 within the input data 802. Each tokenizable element 804 may be converted into a token 806. The set of tokens 806 created from the tokenizable elements 804 of the input data 802 may be sent from the exemplary tokenizer 800 as tokenized output 808. The set of tokens 806 may be such as are used to create the prompt 144 of FIG. 1.

For structured historical data such as plaintext, database, or web-based textual content, tokens may consist of the numerical indexes in an embedded or vectorized (e.g., word2vec or similar) representation of the text content such as are shown here. In some embodiments, a machine learning technique called an autoencoder may be utilized to transform plaintext inputs into high dimensional vectors that are suitable for indexing and tokenization ingestion by the prompt composer 116 introduced with respect to FIG. 1.

In some embodiments, data to be tokenized may include audio, visual, or other multimodal data. For images, video, and similar visual data, tokenization may be performed using a convolution-based tokenizer such as a vision transformer. In some alternate embodiments, multimodal data may be quantized and converted into tokens 806 using a codebook. In yet other alternate embodiments, multimodal data may be directly encoded and for presentation to a language model as a vector space encoding. An exemplary system that utilizes this tokenizer strategy is Gato, a generalist agent capable of ingesting a mixture of discrete and continuous inputs, images, and text as tokens.

FIG. 9A illustrates an isometric view of a BCI headset system 900 in accordance with one embodiment. The BCI headset system 900 comprises an augmented reality display lens 902, a top cover 904, an adjustable strap 906, a padding 908, a ground/reference electrode 910, a ground/reference electrode adjustment dial 912, a biosensor electrodes 914, a battery cell 916, a fit adjustment dial 918, and a control panel cover 920.

The augmented reality display lens 902 may be removable from the top cover 904 as illustrated in FIG. 9C. The augmented reality display lens 902 and top cover 904 may have magnetic portions that facilitate removably securing the augmented reality display lens 902 to the top cover 904. The augmented reality display lens 902 may in one embodiment incorporate a frame around the lens material allowing the augmented reality display lens 902 to be handled without depositing oils on the lens material.

The adjustable strap 906 may secure the BCI headset system 900 to a wearer's head. The adjustable strap 906 may also provide a conduit for connections between the forward housing 932 shown in FIG. 9C and the components located along the adjustable strap 906 and to the rear of the BCI headset system 900. Padding 908 may be located at the front and rear of the BCI headset system 900, as well as along the sides of the adjustable strap 906, as illustrated. A fit adjustment dial 918 at the rear of the BCI headset system 900 may be used to tighten and loosen the fit of the BCI headset system 900 by allowing adjustment to the adjustable strap 906.

A snug fit of the BCI headset system 900 may facilitate accurate readings from the ground/reference electrodes 910 at the sides of the BCI headset system 900, as illustrated here in FIG. 9A as well as in FIG. 9C. A snug fit may also facilitate accurate readings from the biosensor electrodes 914 positioned at the back of the BCI headset system 900. Further adjustment to these sensors may be made using the ground/reference electrode adjustment dials 912 shown, as well as the biosensor electrode adjustment dials 924 illustrated in FIG. 9B.

In addition to the padding 908, biosensor electrodes 914, and fit adjustment dial 918 already described, the rear of the BCI headset system 900 may incorporate a battery cell 916, such as a rechargeable lithium battery pack. A control panel cover 920 may protect additional features when installed, those features being further discussed with respect to FIG. 9B.

FIG. 9B illustrates a rear view of a BCI headset system 900 in accordance with one embodiment. The control panel cover 920 introduced in FIG. 9B is not shown in this figure, so that underlying elements may be illustrated. The BCI headset system 900 further comprises a control panel 922, a biosensor electrode adjustment dials 924, an auxiliary electrode ports 926, and a power switch 928.

With the control panel cover 920 removed, the wearer may access a control panel 922 at the rear of the BCI headset system 900. The control panel 922 may include biosensor electrode adjustment dials 924, which may be used to calibrate and adjust settings for the biosensor electrodes 914 shown in FIG. 9A.

The control panel 922 may also include auxiliary electrode ports 926, such that additional electrodes may be connected to the BCI headset system 900. For example, a set of gloves containing electrodes may be configured to interface with the BCI headset system 900, and readings from the electrodes in the gloves may be sent to the BCI headset system 900 wirelessly, or via a wired connection to the auxiliary electrode ports 926.

The control panel 922 may comprise a power switch 928, allowing the wearer to power the unit on and off while the control panel cover 920 is removed. Replacing the control panel cover 920 may then protect the biosensor electrode adjustment dials 924 and power switch 928 from being accidentally contacted during use. In one embodiment, a power light emitting diode (LED) may be incorporated onto or near the power switch 928 as an indicator of the status of unit power, e.g., on, off, battery low, etc.

FIG. 9C illustrates an exploded view of a BCI headset system 900 in accordance with one embodiment. The BCI headset system 900 further comprises a universal serial bus or USB port 930 in the rear of the BCI headset system 900 as well as a forward housing 932 which may be capable of holding a smart phone 934. The USB port 930 may in one embodiment be a port for a different signal and power connection type. The USB port 930 may facilitate charging of the battery cell 916 and may allow data transfer through connection to additional devices and electrodes.

The top cover 904 may be removed from the forward housing 932 as shown to allow access to the forward housing 932, in order to seat and unseat a smart phone 934. The smart phone 934 may act as all or part of the augmented reality display. In a BCI headset system 900 incorporating a smart phone 934 in this manner, the augmented reality display lens 902 may provide a reflective surface such that a wearer is able to see at least one of the smart phone 934 display and the wearer's surroundings within their field of vision.

The top cover 904 may incorporate a magnetized portion securing it to the forward housing 932, as well as a magnetized lens reception area, such that the augmented reality display lens 902 may, through incorporation of a magnetized frame, be secured in the front of the top cover 904, and the augmented reality display lens 902 may also be removable in order to facilitate secure storage or access to the forward housing 932.

FIG. 9D illustrates an exploded view of a BCI headset system 900 in accordance with one embodiment. The BCI headset system 900 further comprises a smart phone slot 936 in the forward housing 932. When the augmented reality display lens 902 and top cover 904 are removed to expose the forward housing 932 as shown, the smart phone slot 936 may be accessed to allow a smart phone 934 (not shown in this figure) to be inserted.

### Improving Indoor Navigation for a User

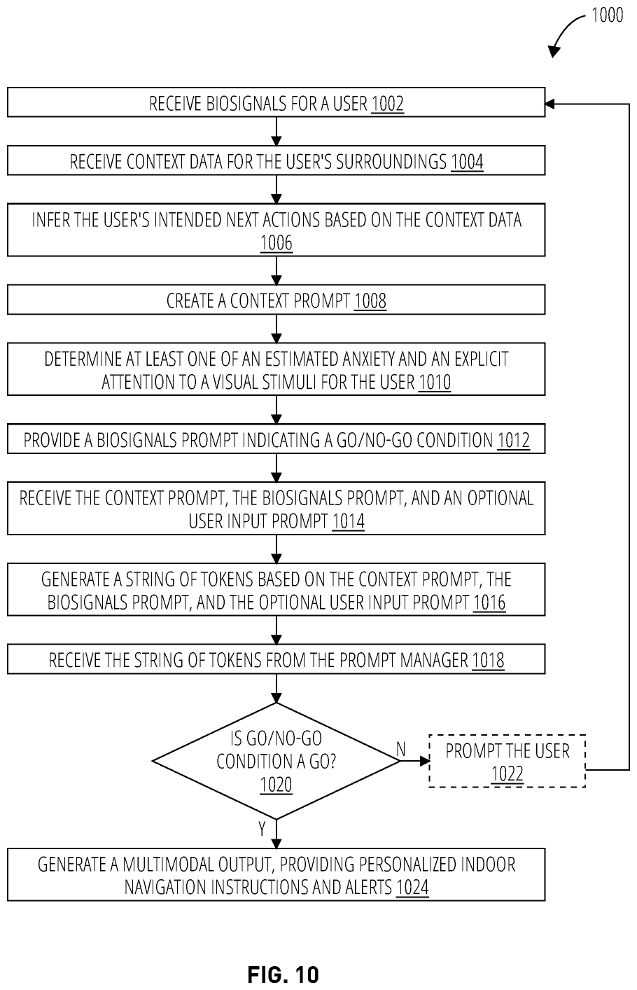

FIG. 10 illustrates an example routine 1000 for improving indoor navigation for a user. Although the example routine 1000 depicts a particular sequence of operations, the sequence may be altered without departing from the scope of the present disclosure. For example, some of the operations depicted may be performed in parallel or in a different sequence that does not materially affect the function of the routine 1000. In other examples, different components of an example device or system that implements the routine 1000 may perform functions at substantially the same time or in a specific sequence.

According to some examples, the method includes receiving biosignals for a user at block 1002. For example, the biosignals subsystem 200 illustrated in FIG. 2 may receive biosignals for a user. The biosignals may include at least one of heart rate, respiratory rate, and gaze direction of the user.

According to some examples, the method includes receiving context data for the user's surroundings at block 1004. For example, the context subsystem 300 illustrated in FIG. 3 may receive context data for the user's surroundings. The context data may include sensor data 110 such as camera data and lidar data capturing elements within the environment surrounding the user. Such sensors may be mounted on a user's wheelchair or onto other wearable peripherals. The context data may also include background material 108 such as indoor floorplan or outside area map data, the user's past routes, and the user's preferred access methods for the environment surrounding the user. In one embodiment, the user's gaze direction and respiratory rate may be inferred from camera data received by the context subsystem 300, rather than from biosignals received by the biosignals subsystem 200.

According to some examples, the method includes inferring the user's intended next actions based on the context data at block 1006. For example, the context subsystem 300 illustrated in FIG. 3 may infer the user's intended next actions based on the context data.

According to some examples, the method includes creating a context prompt at block 1008. For example, the context subsystem 300 illustrated in FIG. 3 may create a context prompt. The context prompt may be a string of tokens corresponding to the user's visually available surroundings, as indicated by the context data.

According to some examples, the method includes determining at least one of an estimated anxiety and an explicit attention to a visual stimuli for the user at block 1010. In other embodiments, the method may estimate other mental states of the user based on available biosensor data, such as excitement, happiness, contentment, etc. For example, the biosignals subsystem 200 illustrated in FIG. 2 may determine at least one of an estimated anxiety and an explicit attention to a visual stimuli for the user. This determination may be made based on the biosignals received for the user.

According to some examples, the method includes providing a biosignals prompt indicating a go/no-go condition at block 1012. For example, the biosignals subsystem 200 illustrated in FIG. 2 may provide a biosignals prompt indicating a go/no-go condition. The go/no-go condition may be determined based on the estimated anxiety exceeding an anxiety threshold or the explicit attention failing to meet an attention threshold. Thus, if a user's anxiety is elevated, or the user is not paying adequate attention, the condition may be determined to be no-go, prioritizing user safety.

According to some examples, the method includes receiving the context prompt, the biosignals prompt, and an optional user input prompt at block 1014. For example, the prompt composer 116 illustrated in FIG. 1 may receive the context prompt, the biosignals prompt, and an optional user input prompt. The user input prompt may indicate a specific user directive, such as “locate the nearest restroom”, “drive to the nearest restroom,” “help me navigate to the nearest restroom,” etc.

According to some examples, the method includes generating a string of tokens based on the context prompt, the biosignals prompt, and the optional user input prompt at block 1016. For example, the prompt composer 116 illustrated in FIG. 1 may generate a string of tokens based on the context prompt, the biosignals prompt, and the optional user input prompt.

According to some examples, the method includes receiving the string of tokens from the prompt manager at block 1018. For example, the GenAI 118 illustrated in FIG. 1 may receive the string of tokens from the prompt manager. The generative AI receiving the string of tokens may be pre-trained to provide navigation guidance.

According to some examples, the method includes determining if the go/no-go condition is a go at decision block 1020. According to some examples, if a go condition is determined, the method includes generating a multimodal output, providing personalized indoor navigation instructions and alerts at block 1024. For example, the GenAI 118 illustrated in FIG. 1 may generate a multimodal output, providing personalized indoor navigation instructions and alerts. The instructions and alerts may be provided to the user as visual, audible, or haptic feedback. In one embodiment, instructions may be provided directly to an autonomous mobility device used by the user, such as a robotic wheelchair, such that the mobility device may perform the indicated navigation tasks without additional user input. In another embodiment, the instructions may be used for navigation in a virtual environment.

If the condition is determined at decision block 1020 not to be a go, (i.e., to be a no-go condition), the GenAI 118 may refrain from generating the multimodal output in one embodiment. In another embodiment, the GenAI 118 may generate a multi-modal output meant to prompt the user in some way at block 1022. The prompt may be an uttered or written statement, a visual, audible, or haptic signal, or some other output designed to calm the user's anxiety, focus the user's attention, or request direct input from the user.

### Controlling Internet of Things (IoT) Devices

FIG. 11 illustrates an example routine 1100 for controlling Internet of Things (IoT) devices with eye blinks, gaze, or other EMG or EOG based signals. Although the example routine 1100 depicts a particular sequence of operations, the sequence may be altered without departing from the scope of the present disclosure. For example, some of the operations depicted may be performed in parallel or in a different sequence that does not materially affect the function of the routine 1100. In other examples, different components of an example device or system that implements the routine 1100 may perform functions at substantially the same time or in a specific sequence.

According to some examples, the method includes receiving biosignals indicative of user eye movements or other muscle movements at block 1102. For example, the biosignals subsystem 200 illustrated in FIG. 2 may receive biosignals indicative of user eye movements. Such biosignals may in one embodiment be derived from cameras imaging a user's eyes coupled to logic capable of detecting and interpreting video data of a user's eyes. Additional sensors may be capable of measuring a user's dept of focus. In one embodiment, such camera and sensor data may be available to the biosignals subsystem 200 as shared data 142 from the context subsystem 300. This information may be translated into a vector that may then be transposed onto a map of the user's surroundings.

According to some examples, the method includes receiving context data including blink patterns and user environment data at block 1104. For example, the context subsystem 300 illustrated in FIG. 3 may receive context data including blink patterns and user environment data. User environment data may include stored maps of the user's known surroundings and the locations of devices within those surroundings, as detected by cameras, lidar sensing, and other sensors, or as stored in a repository of data describing a set of surroundings the user has or may occupy. The context subsystem may use Bluetooth or another discovery protocol to sense local IoT devices. Background material 108 to the context subsystem 300 may include previous commands or protocols issued or fulfilled by the system.

According to some examples, the method includes generating a biosignals prompt based on user eye movements at block 1106. For example, the biosignals subsystem 200 illustrated in FIG. 2 may generate a biosignals prompt based on user eye movements. The biosignals prompt 134 may provide tokenized eye-blink data.

According to some examples, the method includes generating a context prompt based on blink patterns and user environment data at block 1108. For example, the context subsystem 300 illustrated in FIG. 3 may generate a context prompt based on blink patterns and user environment data.

According to some examples, the method includes receiving at least one of the biosignals prompt, the context prompt, and an optional user input prompt at block 1110. For example, the prompt composer 116 illustrated in FIG. 1 may receive at least one of the biosignals prompt, the context prompt, and an optional user input prompt. The user input prompt 138 may include specific IoT commands or partial commands.

According to some examples, the method includes generating a string of tokens based on at least one of the biosignals prompt, the context prompt, and the optional user input prompt at block 1112. For example, the prompt composer 116 illustrated in FIG. 1 may generate a string of tokens based on at least one of the biosignals prompt, the context prompt, and the optional user input prompt.

According to some examples, the method includes receiving the string of tokens from the prompt composer at block 1114. For example, the GenAI 118 illustrated in FIG. 1 may receive the string of tokens from the prompt composer.

According to some examples, the method includes providing the user with multimodal output in the form of personalized device control and automation at block 1116. For example, the GenAI 118 illustrated in FIG. 1 may provide the user with multimodal output in the form of personalized device control and automation. In one embodiment, eye gaze may be used to identify which IoT device the user is looking at and may be combined with contextual information from other sensors (e.g., body temperature) to determine if (for example) the user wants to change the temperature of the room. Temperature sensor adjustment may then be accomplished with eye blink selection or other forms of input, or through automatic adjustment choice and a potential confirmation dialogue. EEG SSVEP based selection controls or a physical switch may also be incorporated to control IoT devices.

### Identifying Conversation Participants

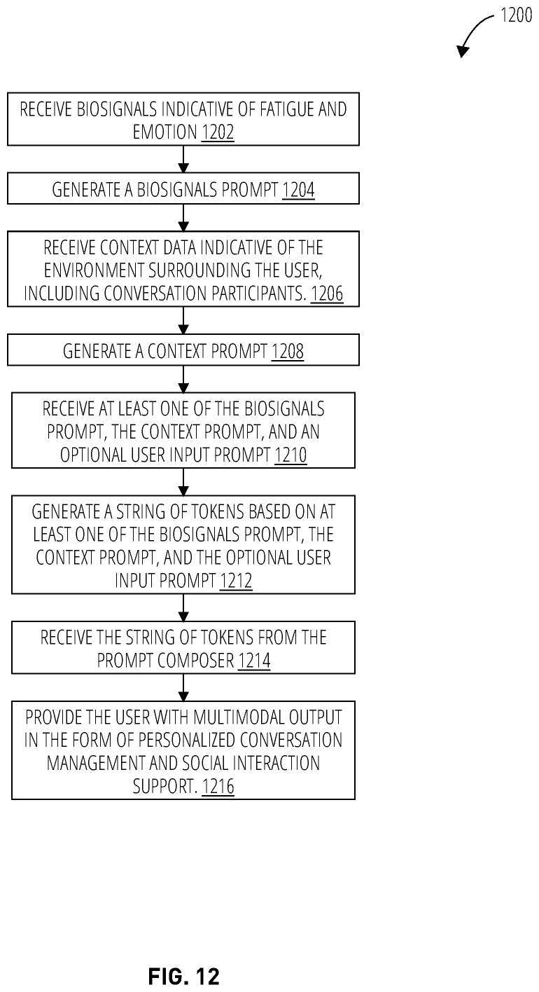

FIG. 12 illustrates an example routine 1200 for identifying conversation participants. Although the example routine 1200 depicts a particular sequence of operations, the sequence may be altered without departing from the scope of the present disclosure. For example, some of the operations depicted may be performed in parallel or in a different sequence that does not materially affect the function of the routine 1200. In other examples, different components of an example device or system that implements the routine 1200 may perform functions at substantially the same time or in a specific sequence.

According to some examples, the method includes receiving biosignals indicative of fatigue and emotion at block 1202. For example, the biosignals subsystem 200 illustrated in FIG. 2 may receive biosignals indicative of fatigue and emotion. These biosignals 106 may include EEG data, heart rate and respiratory rate data, gaze detection data, and other biosignals capable of providing information on a user's energy levels and emotional state.

According to some examples, the method includes generating a biosignals prompt at block 1204. For example, the biosignals subsystem 200 illustrated in FIG. 2 may generate a biosignals prompt. The biosignals prompt 136 may be generated by tokenizing the received biosignals 106.

According to some examples, the method includes receiving context data indicative of the environment surrounding the user, including conversation participants at block 1206. In one embodiment, the system may include a Bluetooth antenna capable of detecting IoT devices in close proximity to the user, although one skilled in the art will readily recognize that other discovery means are also possible. This data may be included in sensor data 110 to the context subsystem 300. Such devices may be used to identify persons in proximity to the user. Camera data may also be used to detect conversation participants, who may be identified through analysis of captured video. Background material 108 to the context subsystem 300 may include previous conversations the user has had with the detected conversation participants, or other details stored about the identified participants. In one embodiment, the system may utilize computational auditory scene analysis (CASA) to identify nearby speakers and associate them with known or unknown contacts. Other possible sensing means may include computer vision, network-based user registration such as in mobile location tracking applications, or calendar attendance entries. As alternative/additional inputs to this system, if a conversation partner has previously been identified, nearby Bluetooth device identifiers (IDs) may be stored as relevant context information, and the system may use a machine learning model or other model type to learn which partner(s) is (are) likely to be present given a particular constellation of Bluetooth device IDs

According to some examples, the method includes generating a context prompt at block 1208. The context subsystem 300 may form a context prompt 138 by tokenizing received context data. The context prompt 138 may indicate an inference of the user's conversation intent based on data such as historical speech patterns and known device identities.

According to some examples, the method includes receiving at least one of the biosignals prompt, the context prompt, and an optional user input prompt at block 1210. For example, the prompt composer 116 illustrated in FIG. 1 may receive at least one of the biosignals prompt, the context prompt, and an optional user input prompt. The user input prompt 140 may include keywords or phrases related to desired speech output.

According to some examples, the method includes generating a string of tokens based on at least one of the biosignals prompt, the context prompt, and the optional user input prompt at block 1212. For example, the prompt composer 116 illustrated in FIG. 1 may generate a string of tokens based on at least one of the biosignals prompt, the context prompt, and the optional user input prompt.

According to some examples, the method includes receiving the string of tokens from the prompt composer at block 1214. For example, the GenAI 118 illustrated in FIG. 1 may receive the string of tokens from the prompt composer. The GenAI 118 may be a pre-trained natural language model configured to provide conversation management and social interaction support.

According to some examples, the method includes providing the user with multimodal output in the form of personalized conversation management and social interaction support at block 1216. For example, the GenAI 118 illustrated in FIG. 1 may provide the user with multimodal output in the form of personalized conversation management and social interaction support. This may come in the form of visual, audible, or haptic feedback provided to the user by the wearable computing and biosignal sensing device 104, as text overlay presented on a computing device, etc.

Incorporating Personal Multimedia Artifacts into a GenAI Corpus

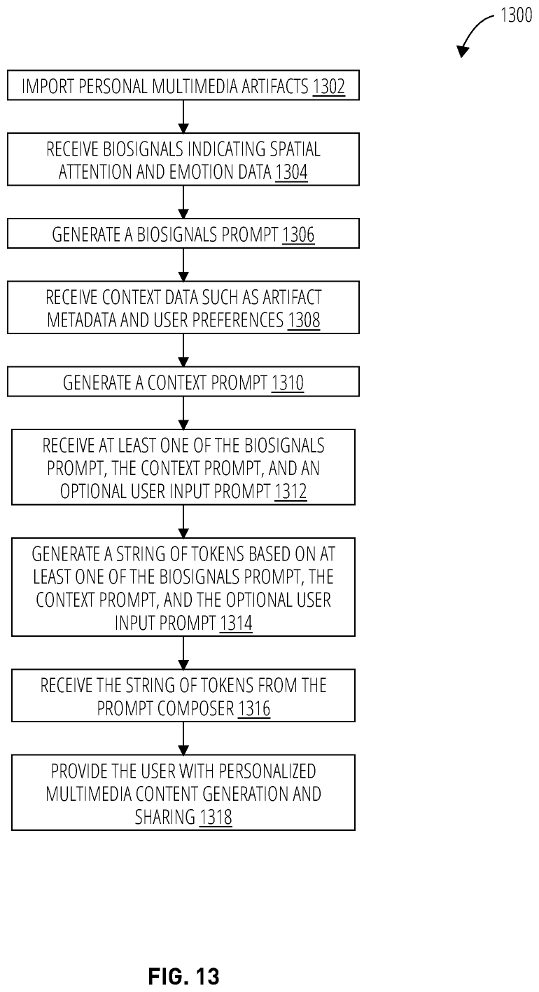

FIG. 13 illustrates an example routine 1300 for incorporating personal multimedia artifacts into a GenAI corpus or model. Although the example routine 1300 depicts a particular sequence of operations, the sequence may be altered without departing from the scope of the present disclosure. For example, some of the operations depicted may be performed in parallel or in a different sequence that does not materially affect the function of the routine 1300. In other examples, different components of an example device or system that implements the routine 1300 may perform functions at substantially the same time or in a specific sequence.

According to some examples, the method includes importing personal multimedia artifacts at block 1302. A media ingestion module may be incorporated into the system, and may be capable of importing personal multimedia artifacts, such as photos and videos, into the GenAI 118.

According to some examples, the method includes receiving biosignals indicating spatial attention and emotion data at block 1304. For example, the biosignals subsystem 200 illustrated in FIG. 2 may receive biosignals indicating spatial attention and emotion data. The biosignals 106 may include EEG data, heart rate and respiratory rate data, gaze detection data, and other data that may be used to detect spatial attention and emotion for the user 102.

According to some examples, the method includes generating a biosignals prompt at block 1306. For example, the biosignals subsystem 200 illustrated in FIG. 2 may generate a biosignals prompt. The biosignals prompt 136 may provide tokenized spatial attention and emotion data.

According to some examples, the method includes receiving context data such as artifact metadata and user preferences at block 1308. For example, the context subsystem 300 illustrated in FIG. 3 may receive context data such as artifact metadata and user preferences. The artifact metadata and user preferences may be stored and provided to the context subsystem 300 as background material 108.

According to some examples, the method includes generating a context prompt at block 1310. For example, the context subsystem 300 illustrated in FIG. 3 may generate a context prompt. The context prompt may be a string of tokens representing an inference of the user's intent with regard to their multimedia artifacts.

According to some examples, the method includes receiving at least one of the biosignals prompt, the context prompt, and an optional user input prompt at block 1312. For example, the prompt composer 116 illustrated in FIG. 1 may receive at least one of the biosignals prompt, the context prompt, and an optional user input prompt. The user input prompt 140 may include names of files and objects in user's multimedia artifact collection, as well as commands for fine-tuning models.

According to some examples, the method includes generating a string of tokens based on at least one of the biosignals prompt, the context prompt, and the optional user input prompt at block 1314. For example, the prompt composer 116 illustrated in FIG. 1 may generate a string of tokens based on at least one of the biosignals prompt, the context prompt, and the optional user input prompt.

According to some examples, the method includes receiving the string of tokens from the prompt composer at block 1316. For example, the GenAI 118 illustrated in FIG. 1 may receive the string of tokens from the prompt composer. In conjunction with the personal multimedia artifacts received in block 1302, the string of tokens may instruct the GenAI 118 in desired categorization and presentation of the user's data within the GenAI 118 corpus.

According to some examples, the method includes providing the user with personalized multimedia content generation and sharing at block 1318. For example, the GenAI 118 illustrated in FIG. 1 may provide the user with personalized multimedia content generation and sharing.

In one embodiment, the user agency and capability augmentation system 100 may infer a set of user actions based on video or photo multimedia artifacts that identify previous actions taken in the same location. Such multimedia artifacts may further include metadata information such as location and time in order to further inform the user's context.

### Emotion Prediction/Estimation

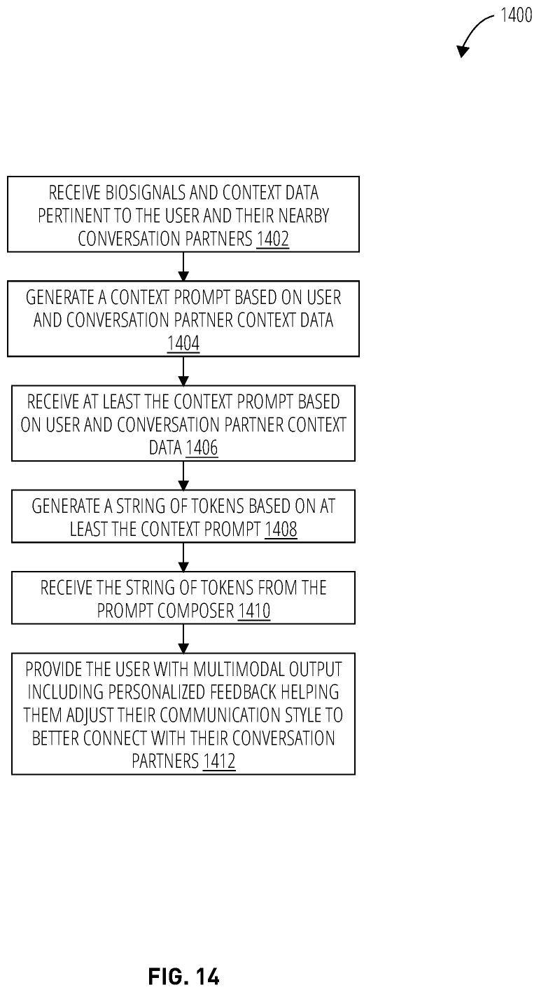

FIG. 14 illustrates an example routine 1400 for emotion prediction/estimation. Although the example routine 1400 depicts a particular sequence of operations, the sequence may be altered without departing from the scope of the present disclosure. For example, some of the operations depicted may be performed in parallel or in a different sequence that does not materially affect the function of the routine 1400. In other examples, different components of an example device or system that implements the routine 1400 may perform functions at substantially the same time or in a specific sequence. The routine 1400 may be performed in whole or part by an AR communication app. This app may be valuable in contexts where individuals with social communication difficulties desire additional support in understanding their conversation partner's feelings. In particular, individuals with cognitive and social disorders such as Autism may utilize this type of information to gain additional feedback on social interactions.

According to some examples, the method includes receiving biosignals and context data pertinent to the user and their nearby conversation partners at block 1402. For example, the context subsystem 300 illustrated in FIG. 3 may receive biosignals and context data pertinent to the user and their nearby conversation partners. The sensor data 110 received by the context subsystem 300 may include phone sensor data, biosignals from the wearable computing and biosignal sensing device 104 or shared data 142 from the biosignals subsystem 200. Background material 108 may be received by the context subsystem 300 which includes data about previous interactions between the user and particular conversation partners. The context subsystem 300 may incorporate a machine learning algorithm to estimate the user's emotions and provide real-time feedback on their conversation partner's emotional state based on facial expressions, body language, and biosignals.

According to some examples, the method includes generating a context prompt based on user and conversation partner context data at block 1404. For example, the context subsystem 300 illustrated in FIG. 3 may generate a context prompt based on user and conversation partner context data. The context prompt 138 may be a tokenization of the biosignals 106 and context data received by the context subsystem 300.

According to some examples, the method includes receiving at least the context prompt based on user and conversation partner context data at block 1406. For example, the prompt composer 116 illustrated in FIG. 1 may receive at least the context prompt based on user and conversation partner context data. The prompt composer 116 may be configured to provide tokens in addition to the tokenized context data. In one embodiment, the user may provide a user input prompt 140 to the prompt composer 116 which may be used to modify the content prompt.

According to some examples, the method includes generating a string of tokens based on at least the context prompt at block 1408. For example, the prompt composer 116 illustrated in FIG. 1 may generate a string of tokens based on at least the context prompt.

According to some examples, the method includes receiving the string of tokens from the prompt composer at block 1410. For example, the GenAI 118 illustrated in FIG. 1 may receive the string of tokens from the prompt composer.

According to some examples, the method includes providing the user with multimodal output including personalized feedback helping them adjust their communication style to better connect with their conversation partners at block 1412. For example, the GenAI 118 illustrated in FIG. 1 may provide the user with multimodal output including personalized feedback helping them adjust their communication style to better connect with their conversation partners. The large multimodal output stage 120 may include a set of suggestions that are rendered into private audio for the user 102. In another embodiment, the output is visually rendered into a heads-up display. In yet another embodiment, the output may be rendered into a multimodal feedback that expresses an affect grid coordinate to the user using a mixture of output modalities (e.g., valence may be mapped to an auditory pattern and arousal may be mapped to a haptic pattern). In one embodiment, the system may further provide context information (e.g., background material 108) summarizing prior engagements with specific individuals and proposing suggestions for improving social outcomes.

### Enhancing Reading Comprehension

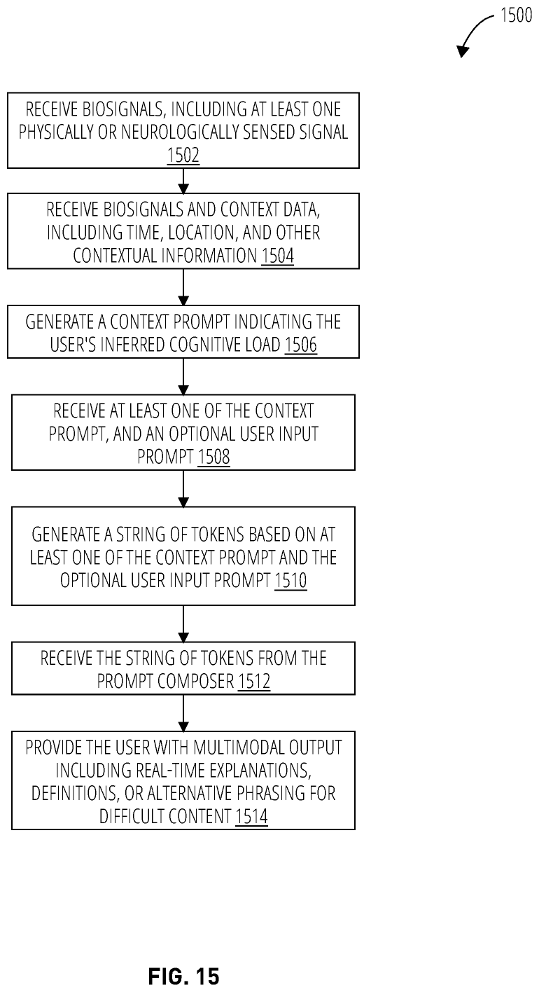

FIG. 15 illustrates an example routine 1500 for enhancing reading comprehension. Although the example routine 1500 depicts a particular sequence of operations, the sequence may be altered without departing from the scope of the present disclosure. For example, some of the operations depicted may be performed in parallel or in a different sequence that does not materially affect the function of the routine 1500. In other examples, different components of an example device or system that implements the routine 1500 may perform functions at substantially the same time or in a specific sequence.

According to some examples, the method includes receiving biosignals, including at least one physically sensed signal or neurologically sensed signal at block 1502. For example, the biosignals subsystem 200 illustrated in FIG. 2 may receive biosignals, including at least one physically sensed signal or neurologically sensed signal. The biosignals 106 may be detected using the wearable computing and biosignal sensing device 104 or additional biosensors 202 included in the biosignals subsystem 200. The biosignals 106 may include EEG data, heart and respiratory rate data, or other data that may be indicative of a user's cognitive load.

According to some examples, the method includes receiving biosignals and context data, including time, location, and other contextual information at block 1504. For example, the context subsystem 300 illustrated in FIG. 3 may receive biosignals and context data, including time, location, and other contextual information. The context subsystem 300 may receive biosignals 106 as shared data 142 from the biosignals subsystem 200.

According to some examples, the method includes generating a context prompt indicating the user's inferred cognitive load at block 1506. For example, the context subsystem 300 illustrated in FIG. 3 may generate a context prompt indicating the user's inferred cognitive load. The context prompt 138 may be generated by processing the context data and biosignals 106 into tokens.

According to some examples, the method includes receiving at least one of the context prompt, and an optional user input prompt at block 1508. For example, the prompt composer 116 illustrated in FIG. 1 may receive at least one of the context prompt, and an optional user input prompt. The user input prompt 140 may include requests for reading comprehension assistance and preferences for how such assistance is provided.

According to some examples, the method includes generating a string of tokens based on at least one of the context prompt and the optional user input prompt at block 1510. For example, the prompt composer 116 illustrated in FIG. 1 may generate a string of tokens based on at least one of the context prompt and the optional user input prompt.

According to some examples, the method includes receiving the string of tokens from the prompt composer at block 1512. For example, the GenAI 118 illustrated in FIG. 1 may receive the string of tokens from the prompt composer. The GenAI 118 may be a pre-trained natural language model that may utilize the prompt to provide real-time explanations, definitions, or alternative phrasing for difficult or unfamiliar content.

According to some examples, the method includes providing the user with multimodal output including real-time explanations, definitions, or alternative phrasing for difficult or unfamiliar content at block 1514. For example, the GenAI 118 illustrated in FIG. 1 may provide the user with multimodal output including real-time explanations, definitions, or alternative phrasing for difficult or unfamiliar content. The multimodal output may be feedback communicated to the user via audio or using a virtual and augmented reality visual system that overlays explanations or hyperlinks on the reading materials. In another embodiment, the feedback may communicate the user's mental state back to the user via a multimodal sensation in order to enable conscious recognition of their mental state as a form of biofeedback. The system implementing routine 1500 may use biosignals and user state to determine which sections of content the user is having difficulty with, such as detecting sections the user has to read more than once, sections that contain content semantically similar (based on LLM semantic analysis) to those that the model has previously determined were difficult, etc.

### Enhancing Autonomous Vehicle Safety and Comfort

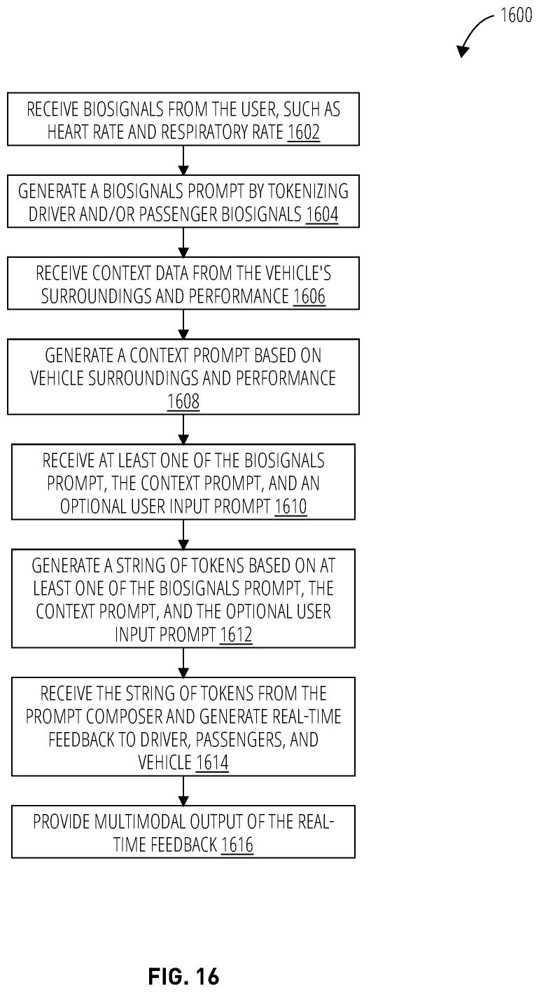

FIG. 16 illustrates an example routine 1600 for enhancing autonomous vehicle safety and comfort. Although the example routine 1600 depicts a particular sequence of operations, the sequence may be altered without departing from the scope of the present disclosure. For example, some of the operations depicted may be performed in parallel or in a different sequence that does not materially affect the function of the routine 1600. In other examples, different components of an example device or system that implements the routine 1600 may perform functions at substantially the same time or in a specific sequence.

According to some examples, the method includes receiving biosignals from the user, such as heart rate and respiratory rate at block 1602. For example, the biosignals subsystem 200 illustrated in FIG. 2 may receive biosignals from the user, such as EEG (event-related potential or similar), heart rate and respiratory rate. The biosignals 106 may be detected using a wearable computing and biosignal sensing device 104 or the additional biosensors 202 configured in the biosignals subsystem 200. In one embodiment, biosensors may be integrated into portions of the vehicle and may detect biosignals 106 from the driver and any passengers present.

According to some examples, the method includes generating a biosignals prompt by tokenizing driver and/or passenger biosignals at block 1604. For example, the biosignals subsystem 200 illustrated in FIG. 2 may generate a biosignals prompt by tokenizing driver and/or passenger biosignals. The biosignals prompt 136 may be generated by tokenizing the received biosignals 106.

According to some examples, the method includes receiving context data from the vehicle's surroundings and performance at block 1606. For example, the context subsystem 300 illustrated in FIG. 3 may receive context data from the vehicle's surroundings and performance. Such data may be in the form of sensor data 110 from cameras and lidar, in addition to other device data 112 from the vehicle's computerized vehicle management unit.

According to some examples, the method includes generating a context prompt based on vehicle surroundings and performance at block 1608. For example, the context subsystem 300 illustrated in FIG. 3 may generate a context prompt based on vehicle surroundings and performance.

According to some examples, the method includes receiving at least one of the biosignals prompt, the context prompt, and an optional user input prompt at block 1610. For example, the prompt composer 116 illustrated in FIG. 1 may receive at least one of the biosignals prompt, the context prompt, and an optional user input prompt. The user input prompt 140 may include data from vehicle passengers.

According to some examples, the method includes generating a string of tokens based on at least one of the biosignals prompt, the context prompt, and the optional user input prompt at block 1612. For example, the prompt composer 116 illustrated in FIG. 1 may generate a string of tokens based on at least one of the biosignals prompt, the context prompt, and the optional user input prompt.

According to some examples, the method includes receiving the string of tokens from the prompt composer and generating real-time feedback to driver, passengers, and vehicle at block 1614. For example, the GenAI 118 illustrated in FIG. 1 may receive the string of tokens from the prompt composer and generate real-time feedback to driver, passengers, and vehicle.

According to some examples, the method includes providing multimodal output of the real-time feedback at block 1616. For example, the GenAI 118 illustrated in FIG. 1 may provide multimodal output of the real-time feedback. In one embodiment, the real-time feedback may instruct the vehicle's autonomous control system to adjust speed, route, and other factors to improve passenger safety and comfort. The output of the GenAI 118 may be converted into multimodal sensations through vehicle instruments such as the steering wheel, driver heads up display and/or over the in-vehicle audio system. In one embodiment the system may utilize estimates of the user's anxiety or comfort, derived from one or more biosensors, in order to adapt the navigation or driving style of an autonomous vehicle.

### Personalizing E-Commerce Experience

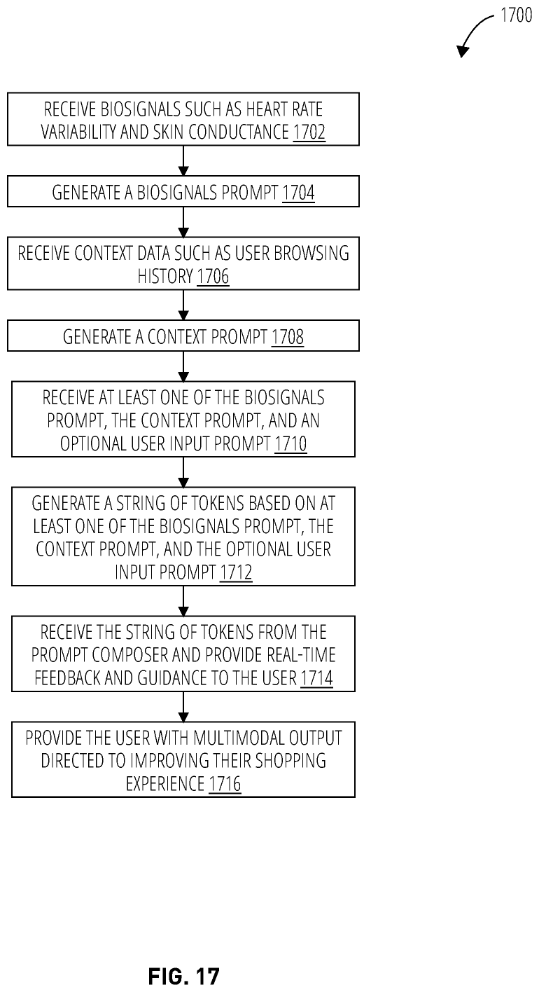

FIG. 17 illustrates an example routine 1700 for personalizing e-commerce experience. Although the example routine 1700 depicts a particular sequence of operations, the sequence may be altered without departing from the scope of the present disclosure. For example, some of the operations depicted may be performed in parallel or in a different sequence that does not materially affect the function of the routine 1700. In other examples, different components of an example device or system that implements the routine 1700 may perform functions at substantially the same time or in a specific sequence.

According to some examples, the method includes receiving biosignals such as heart rate variability and skin conductance at block 1702. For example, the biosignals subsystem 200 illustrated in FIG. 2 may receive biosignals such as heart rate variability and skin conductance.

According to some examples, the method includes generating a biosignals prompt at block 1704. For example, the biosignals subsystem 200 illustrated in FIG. 2 may generate a biosignals prompt. The biosignals prompt 134 may be generated by tokenizing the received biosignals 106.

According to some examples, the method includes receiving context data such as user browsing history at block 1706. For example, the context subsystem 300 illustrated in FIG. 3 may receive context data such as user browsing history.

According to some examples, the method includes generating a context prompt at block 1708. For example, the context subsystem 300 illustrated in FIG. 3 may generate a context prompt.

According to some examples, the method includes receiving at least one of the biosignals prompt, the context prompt, and an optional user input prompt at block 1710. For example, the prompt composer 116 illustrated in FIG. 1 may receive at least one of the biosignals prompt, the context prompt, and an optional user input prompt. The user input prompt 138 may include keywords, phrases, or other input directly entered by the user.

According to some examples, the method includes generating a string of tokens based on at least one of the biosignals prompt, the context prompt, and the optional user input prompt at block 1712. For example, the prompt composer 116 illustrated in FIG. 1 may generate a string of tokens based on at least one of the biosignals prompt, the context prompt, and the optional user input prompt.

According to some examples, the method includes receiving the string of tokens from the prompt composer and provide real-time feedback and guidance to the user at block 1714. For example, the GenAI 118 illustrated in FIG. 1 may receive the string of tokens from the prompt composer and provide real-time feedback and guidance to the user. The feedback and guidance may be directed toward improving the user's shopping experience.

According to some examples, the method includes providing the user with multimodal output directed to improving their shopping experience at block 1716. For example, the GenAI 118 illustrated in FIG. 1 may provide the user with multimodal output directed to improving their shopping experience. This multimodal output may include personalized product recommendations and checkout optimization. The personalized feedback generated by the generative AI 118 may be converted into visual, auditory or haptic means to be rendered to the user. In a related embodiment, the sensed biosignals 106 information may be utilized by the e-commerce platform to test relevance of product or service recommendations to the user 102. In yet other embodiments, an e-commerce platform may utilize this feedback mechanism to conduct A/B testing of new product or service offerings.

### Enhancing Traveler Experience

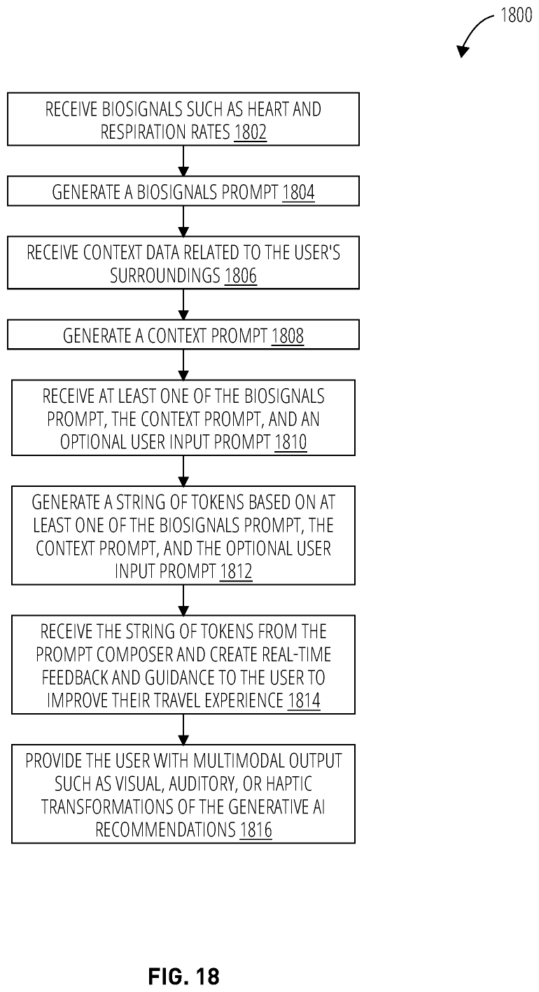

FIG. 18 illustrates an example routine 1800 for enhancing traveler experience. Although the example routine 1800 depicts a particular sequence of operations, the sequence may be altered without departing from the scope of the present disclosure. For example, some of the operations depicted may be performed in parallel or in a different sequence that does not materially affect the function of the routine 1800. In other examples, different components of an example device or system that implements the routine 1800 may perform functions at substantially the same time or in a specific sequence.

According to some examples, the method includes receiving biosignals such as heart and respiration rates at block 1802. For example, the biosignals subsystem 200 illustrated in FIG. 2 may receive biosignals such as heart and respiration rates.

According to some examples, the method includes generating a biosignals prompt at block 1804. For example, the biosignals subsystem 200 illustrated in FIG. 2 may generate a biosignals prompt. The biosignals prompt 136 may be generated by tokenizing the received biosignals 106.

According to some examples, the method includes receiving context data related to the user's surroundings at block 1806. For example, the context subsystem 300 illustrated in FIG. 3 may receive context data related to the user's surroundings. Context data may include sensor data 110 such as audio and video data capturing body language and spoken words from a user's conversation partner, or written language from signs or menus near the user. Context data may include other device data 112 such as the user's location as provided from a smartphone global positioning system (GPS) application. Context data may include background material 108 such as the user's foreign language progress in a language app, the user's dining preferences, the user's interests, etc.

According to some examples, the method includes generating a context prompt at block 1808. For example, the context subsystem 300 illustrated in FIG. 3 may generate a context prompt. The context prompt 138 may be generated by tokenizing the received context data.

According to some examples, the method includes receiving at least one of the biosignals prompt, the context prompt, and an optional user input prompt at block 1810. For example, the prompt composer 116 illustrated in FIG. 1 may receive at least one of the biosignals prompt, the context prompt, and an optional user input prompt. The user input prompt 140 may include keywords or phrases related to the user's immediate need for navigation, translation, or point of interest suggestions.

According to some examples, the method includes generating a string of tokens based on at least one of the biosignals prompt, the context prompt, and the optional user input prompt at block 1812. For example, the prompt composer 116 illustrated in FIG. 1 may generate a string of tokens based on at least one of the biosignals prompt, the context prompt, and the optional user input prompt.

According to some examples, the method includes receiving the string of tokens from the prompt composer and creating real-time feedback and guidance to the user to improve their travel experience at block 1814. For example, the GenAI 118 illustrated in FIG. 1 may receive the string of tokens from the prompt composer and create real-time feedback and guidance to the user to improve their travel experience. Feedback may include personalized recommendations for activities, dining, and accommodations.

According to some examples, the method includes providing the user with multimodal output such as visual, auditory, or haptic transformations of the generative AI recommendations at block 1816. For example, the GenAI 118 illustrated in FIG. 1 may provide the user with multimodal output such as visual, auditory, or haptic transformations of the generative AI recommendations. In this manner, the system may automatically translate language between the user and their conversation partner(s) or written text in the environment and may adapt translations to the user's knowledge and state. In some embodiments, the multimodal output may be automatically translated into a local language or into a precise utterance 124 that is easier for the conversation partner to understand. The GenAI 118, in one embodiment an LLM, may utilize additional cloud-based resources to facilitate this translation function, including connecting to travel related booking services on the user's behalf in order to provide concrete option planning for the user 102.

### Personalized Rest and Activity Recommendations

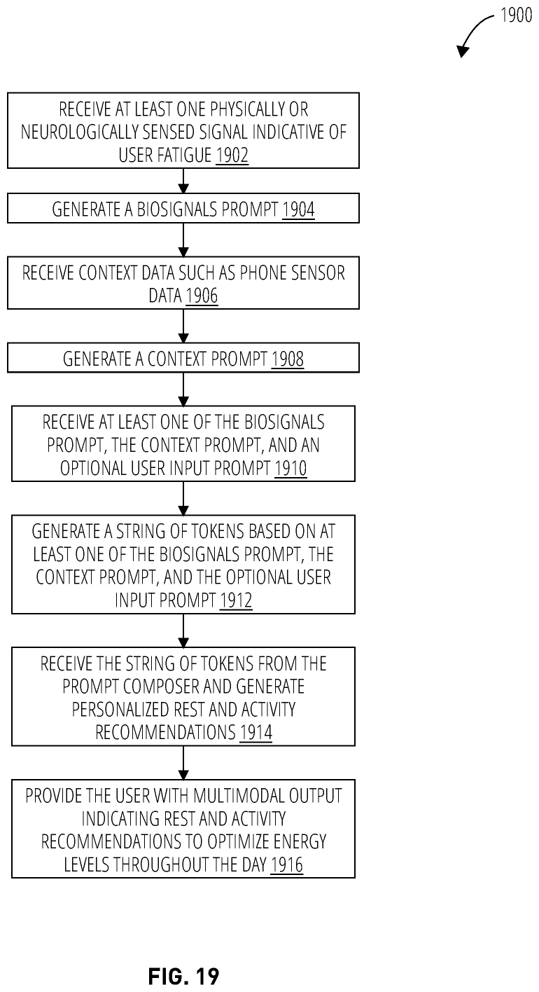

FIG. 19 illustrates an example routine 1900 for providing personalized rest and activity recommendations. Although the example routine 1900 depicts a particular sequence of operations, the sequence may be altered without departing from the scope of the present disclosure. For example, some of the operations depicted may be performed in parallel or in a different sequence that does not materially affect the function of the routine 1900. In other examples, different components of an example device or system that implements the routine 1900 may perform functions at substantially the same time or in a specific sequence.

According to some examples, the method includes receiving at least one physically sensed signal or neurologically sensed signal indicative of user fatigue at block 1902. For example, the biosignals subsystem 200 illustrated in FIG. 2 may receive at least one physically sensed signal or neurologically sensed signal indicative of user fatigue. These signals may include but are not limited to biosignals 106 such as heart rate, skin conductance, and muscle tension. The biosignals subsystem 200 in this embodiment may also infer the user's fatigue level utilizing EEG, heart rate, and similar biosignals 106.

According to some examples, the method includes generating a biosignals prompt at block 1904. For example, the biosignals subsystem 200 illustrated in FIG. 2 may generate a biosignals prompt. The biosignals prompt 136 may be generated by tokenizing the received biosignals 106.

According to some examples, the method includes receiving context data such as phone sensor data at block 1906. For example, the context subsystem 300 illustrated in FIG. 3 may receive context data such as sensor data. Sensor data 110 from a smartphone may indicate the user's physical movement, as well as smartphone usage patterns, which may include past behaviors.

According to some examples, the method includes generating a context prompt at block 1908. For example, the context subsystem 300 illustrated in FIG. 3 may generate a context prompt.

According to some examples, the method includes receiving at least one of the biosignals prompt, the context prompt, and an optional user input prompt at block 1910. For example, the prompt composer 116 illustrated in FIG. 1 may receive at least one of the biosignals prompt, the context prompt, and an optional user input prompt. The user input prompt 140 may include keywords or phrases related to desired real-time feedback.

According to some examples, the method includes generating a string of tokens based on at least one of the biosignals prompt, the context prompt, and the optional user input prompt at block 1912. For example, the prompt composer 116 illustrated in FIG. 1 may generate a string of tokens based on at least one of the biosignals prompt, the context prompt, and the optional user input prompt.

According to some examples, the method includes receiving the string of tokens from the prompt composer and generating personalized rest and activity recommendations at block 1914. For example, the GenAI 118 illustrated in FIG. 1 may receive the string of tokens from the prompt composer and generate personalized rest and activity recommendations. The GenAI 118 may be a pre-trained machine learning model configured to utilize the prompt to generate personalized rest and activity recommendations for the user 102 to optimize their energy levels throughout the day or progress over the long term.

According to some examples, the method includes providing the user with multimodal output indicating rest and activity recommendations to optimize energy levels throughout the day at block 1916. For example, the GenAI 118 illustrated in FIG. 1 may provide the user with multimodal output indicating rest and activity recommendations to optimize energy levels throughout the day. Feedback may be provided using visual, auditory or haptic transformations of the GenAI 118 multimodal outputs. The system may be valuable in contexts where individuals desire support in managing their fatigue levels and optimizing energy levels throughout the day, such as individuals with chronic fatigue syndrome, those in high-stress work environments, or distance athletes. In other embodiments, the system further provides the user with nutrition, shopping, and scheduling guidance in order to enhance the user's sense of well being.

### Optimizing AR/VR Workspace Productivity

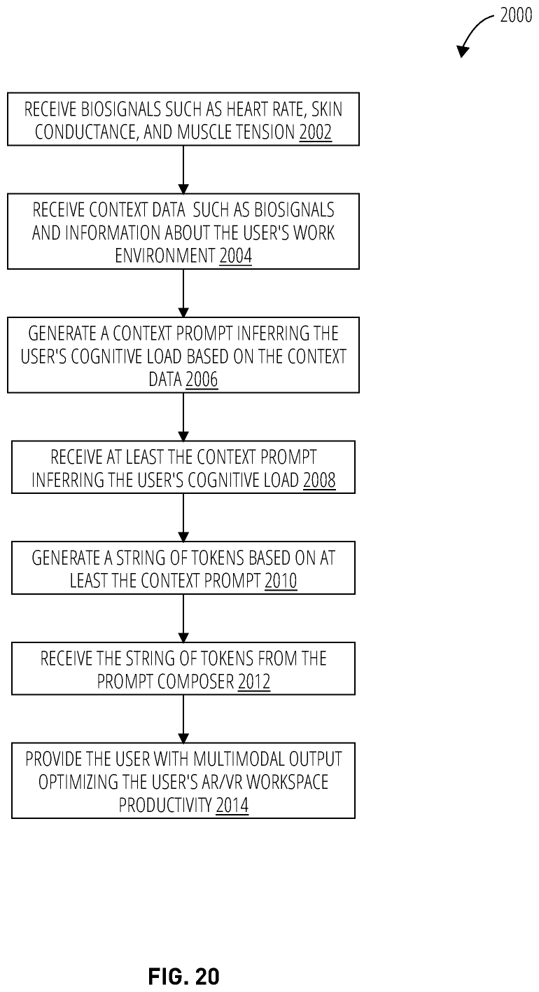

FIG. 20 illustrates an example routine 2000 for optimizing AR/VR workspace productivity. Although the example routine 2000 depicts a particular sequence of operations, the sequence may be altered without departing from the scope of the present disclosure. For example, some of the operations depicted may be performed in parallel or in a different sequence that does not materially affect the function of the routine 2000. In other examples, different components of an example device or system that implements the routine 2000 may perform functions at substantially the same time or in a specific sequence.

According to some examples, the method includes receiving biosignals such as heart rate, skin conductance, and muscle tension at block 2002. For example, the biosignals subsystem 200 illustrated in FIG. 2 may receive biosignals such as heart rate, skin conductance, and muscle tension.

According to some examples, the method includes receiving context data such as biosignals and information about the user's work environment at block 2004. For example, the context subsystem 300 illustrated in FIG. 3 may receive context data such as biosignals and information about the user's work environment. Biosignals 106 may be provided as shared data 142 from the 200. The user's work environment may be represented by sensor data 110 capturing characteristics of the user's surroundings, such as light levels, noise levels, and temperature. The user's work environment may also be characterized by application context 114 provided by computing applications the user is currently working in.

According to some examples, the method includes generating a context prompt inferring the user's cognitive load based on the context data at block 2006. For example, the context subsystem 300 illustrated in FIG. 3 may generate a context prompt inferring or measuring the user's cognitive load based on the context data. The context prompt may include the biosignal and context data processed into tokens.

According to some examples, the method includes receiving at least the context prompt inferring the user's cognitive load at block 2008. For example, the prompt composer 116 illustrated in FIG. 1 may receive at least the context prompt inferring the user's cognitive load. In some embodiments, the prompt composer 116 may further receive a user input prompt 140 conveying the user's perceived state of mind and energy levels.

According to some examples, the method includes generating a string of tokens based on at least the context prompt at block 2010. For example, the prompt composer 116 illustrated in FIG. 1 may generate a string of tokens based on at least the context prompt. The user input prompt 140 may also be tokenized and included in the prompt 144.

According to some examples, the method includes receiving the string of tokens from the prompt composer at block 2012. For example, the GenAI 118 illustrated in FIG. 1 may receive the string of tokens from the prompt composer. The GenAI 118 may be configured to generate computer interpretable codes instead of or in addition to natural language or haptic feedback to the user.

According to some examples, the method includes providing the user with multimodal output optimizing the user's AR/VR workspace productivity at block 2014. For example, the GenAI 118 illustrated in FIG. 1 may provide the user with multimodal output optimizing the user's AR/VR workspace productivity. The GenAI 118 may be prompted to provide computer interpretable codes that modify or change the layout of the VR/AR, desktop, or physical workspace. The system may be valuable in contexts where individuals desire support to manage their cognitive load in virtual workspaces, such as remote workers or individuals with cognitive disabilities.

### Enhancing Social Media Engagement

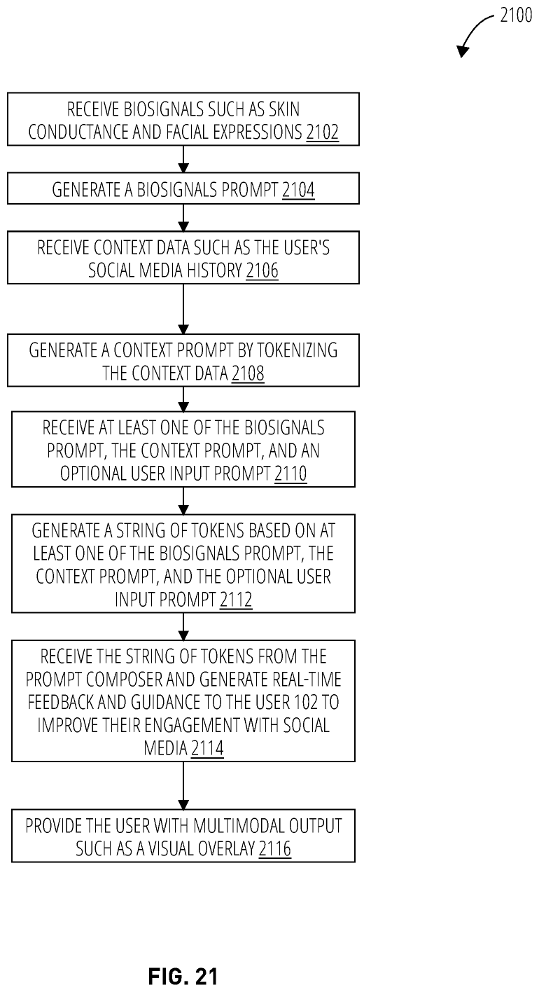

FIG. 21 illustrates an example routine 2100 for enhancing social media engagement and/or assisting a user 102 in achieving social goals. Although the example routine 2100 depicts a particular sequence of operations, the sequence may be altered without departing from the scope of the present disclosure. For example, some of the operations depicted may be performed in parallel or in a different sequence that does not materially affect the function of the routine 2100. In other examples, different components of an example device or system that implements the routine 2100 may perform functions at substantially the same time or in a specific sequence.

According to some examples, the method includes receiving biosignals such as skin conductance and facial expressions at block 2102. For example, the biosignals subsystem 200 illustrated in FIG. 2 may receive biosignals such as skin conductance and facial expressions.

According to some examples, the method includes generating a biosignals prompt at block 2104. For example, the biosignals subsystem 200 illustrated in FIG. 2 may generate a biosignals prompt. The biosignals prompt 136 may be generated by tokenizing the received biosignals 106.

According to some examples, the method includes receiving context data such as the user's social media history at block 2106. For example, the context subsystem 300 illustrated in FIG. 3 may receive context data such as the user's social media history.

According to some examples, the method includes generating a context prompt by tokenizing the context data at block 2108. For example, the context subsystem 300 illustrated in FIG. 3 may generate a context prompt by tokenizing the context data.

According to some examples, the method includes receiving at least one of the biosignals prompt, the context prompt, and an optional user input prompt at block 2110. For example, the prompt composer 116 illustrated in FIG. 1 may receive at least one of the biosignals prompt, the context prompt, and an optional user input prompt. The user input prompt 140 may include keywords or commands from the user indicating enhancements they desire or a self-assessment of their emotional state.

According to some examples, the method includes generating a string of tokens based on at least one of the biosignals prompt, the context prompt, and the optional user input prompt at block 2112. For example, the prompt composer 116 illustrated in FIG. 1 may generate a string of tokens based on at least one of the biosignals prompt, the context prompt, and the optional user input prompt.

According to some examples, the method includes receiving the string of tokens from the prompt composer and generating real-time feedback and guidance to the user 102 to improve their engagement with social media at block 2114. For example, the GenAI 118 illustrated in FIG. 1 may receive the string of tokens from the prompt composer and generate real-time feedback and guidance to the user 102 to improve their engagement with social media. Such guidance may include personalized recommendations for content, real-time feedback on the user's emotional state, and suggestions for alternate phrasing of posts.

According to some examples, the method includes providing the user with multimodal output such as a visual overlay at block 2116. For example, the GenAI 118 illustrated in FIG. 1 may provide the user 102 with multimodal output such as a visual overlay. In some embodiments the feedback may be provided via transformation of the of biosignals 106 and a valence estimate based on the user's generated content. This feedback may be in the form of visual, auditory or haptic sensations that are displayed, rendered, or otherwise generated on a wearable or other computing interface device.

### Enhancing Workplace Safety

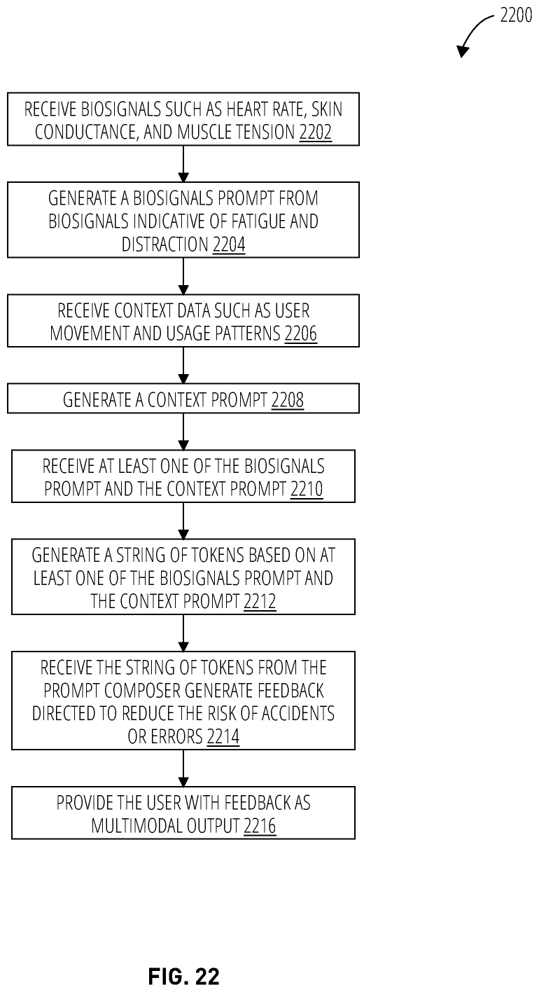

FIG. 22 illustrates an example routine 2200 for enhancing workplace safety. Although the example routine 2200 depicts a particular sequence of operations, the sequence may be altered without departing from the scope of the present disclosure. For example, some of the operations depicted may be performed in parallel or in a different sequence that does not materially affect the function of the routine 2200. In other examples, different components of an example device or system that implements the routine 2200 may perform functions at substantially the same time or in a specific sequence.

According to some examples, the method includes receiving biosignals such as heart rate, skin conductance, and muscle tension at block 2202. For example, the biosignals subsystem 200 illustrated in FIG. 2 may receive biosignals such as heart rate, skin conductance, and muscle tension. Biosignals 106 may include at least one mental and/or physically sensed signal and may be sensed by the wearable computing and biosignal sensing device 104 or the additional biosensors 202 incorporated in the biosignals subsystem 200. Biosignals 106 may also be available from other wearable or handheld device sensors.

According to some examples, the method includes generating a biosignals prompt from biosignals indicative of fatigue and distraction at block 2204. For example, the biosignals subsystem 200 illustrated in FIG. 2 may generate a biosignals prompt from biosignals indicative of fatigue and distraction. The biosignals prompt 136 may be generated by tokenizing the received biosignals 106.

According to some examples, the method includes receiving context data such as user movement and usage patterns at block 2206. For example, the context subsystem 300 illustrated in FIG. 3 may receive context data such as user movement and usage patterns. Such context data may be available as sensor data 110 and application context 114 from wearable or handheld smart devices.

According to some examples, the method includes generating a context prompt at block 2208. For example, the context subsystem 300 illustrated in FIG. 3 may generate a context prompt.

According to some examples, the method includes receiving at least one of the biosignals prompt and the context prompt at block 2210. For example, the prompt composer 116 illustrated in FIG. 1 may receive at least one of the biosignals prompt and the context prompt.

According to some examples, the method includes generating a string of tokens based on at least one of the biosignals prompt and the context prompt at block 2212. For example, the prompt composer 116 illustrated in FIG. 1 may generate a string of tokens based on at least one of the biosignals prompt and the context prompt.

According to some examples, the method includes receiving the string of tokens from the prompt composer generating feedback directed to reduce the risk of accidents or errors at block 2214. For example, the GenAI 118 illustrated in FIG. 1 may receive the string of tokens from the prompt composer and generate feedback directed to reduce the risk of accidents or errors.

According to some examples, the method includes providing the user with feedback as multimodal output at block 2216. For example, the GenAI 118 illustrated in FIG. 1 may provide the user with feedback as multimodal output. The multimodal output stage 120 may provide visual, auditory or haptic stimulation. The feedback may provide alerts, prompts, or recommendations for breaks, rest, or changes in work conditions in augmented reality to reduce the risk of accidents or errors. In some embodiments, the user agency and capability augmentation system 100 may further generate summary reports or recommendations to others such as managers or colleagues in order to facilitate workplace optimization.

### Reducing Social Isolation

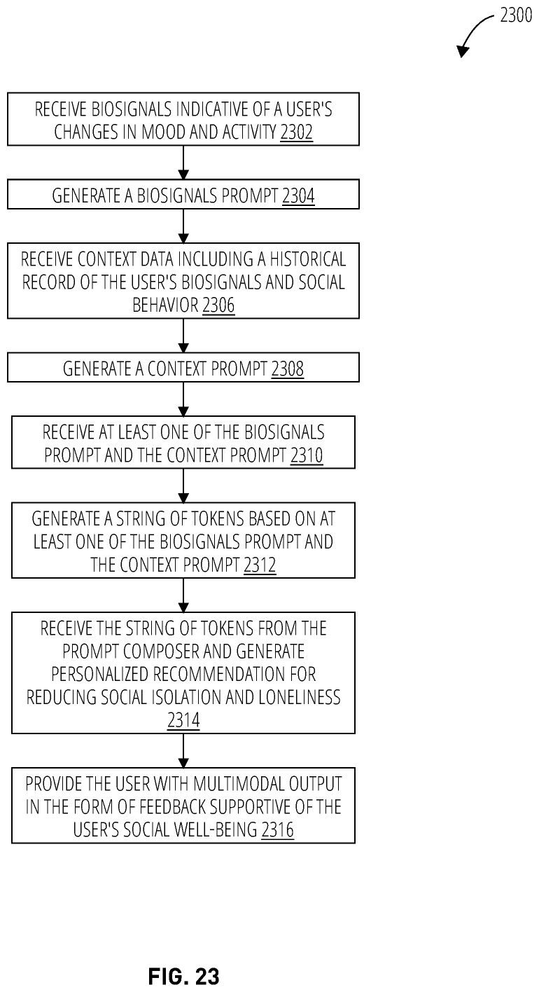

FIG. 23 illustrates an example routine 2300 for reducing social isolation. Although the example routine 2300 depicts a particular sequence of operations, the sequence may be altered without departing from the scope of the present disclosure. For example, some of the operations depicted may be performed in parallel or in a different sequence that does not materially affect the function of the routine 2300. In other examples, different components of an example device or system that implements the routine 2300 may perform functions at substantially the same time or in a specific sequence.

According to some examples, the method includes receiving biosignals indicative of a user's changes in mood and activity at block 2302. For example, the biosignals subsystem 200 illustrated in FIG. 2 may receive biosignals indicative of a user's changes in mood and activity.

According to some examples, the method includes generating a biosignals prompt at block 2304. For example, the biosignals subsystem 200 illustrated in FIG. 2 may generate a biosignals prompt. The biosignals prompt 136 may be generated by tokenizing the received biosignals 106.

According to some examples, the method includes receiving context data including a historical record of the user's biosignals and social behavior at block 2306. For example, the context subsystem 300 illustrated in FIG. 3 may receive context data including a historical record of the user's biosignals and social behavior. The user's biosignals 106 history may be tracked by storing as background material 108 the biosignals 106 received from the wearable computing and biosignal sensing device 104 and additional biosensors 202, which may be shared to the context subsystem 300 as shared data 142 from the biosignals subsystem 200. The user's history of social behavior and activity on social media may be tracked through the online record and received as application context 114 and/or background material 108.

According to some examples, the method includes generating a context prompt at block 2308. For example, the context subsystem 300 illustrated in FIG. 3 may generate a context prompt. The context prompt 138 may be generated by tokenizing the received context data. The context prompt 138 may indicate predictions for changes in the user's mood, activity, and social behavior.

According to some examples, the method includes receiving at least one of the biosignals prompt and the context prompt at block 2310. For example, the prompt composer 116 illustrated in FIG. 1 may receive at least one of the biosignals prompt and the context prompt.

According to some examples, the method includes generating a string of tokens based on at least one of the biosignals prompt and the context prompt at block 2312. For example, the prompt composer 116 illustrated in FIG. 1 may generate a string of tokens based on at least one of the biosignals prompt and the context prompt.

According to some examples, the method includes receiving the string of tokens from the prompt composer and generating personalized recommendation for reducing social isolation and loneliness at block 2314. For example, the GenAI 118 illustrated in FIG. 1 may receive the string of tokens from the prompt composer and generate personalized recommendation for reducing social isolation and loneliness.

According to some examples, the method includes providing the user with multimodal output in the form of feedback supportive of the user's social well-being at block 2316. For example, the GenAI 118 illustrated in FIG. 1 may provide the user with multimodal output in the form of feedback supportive of the user's social well-being. This feedback may be provided in the form of visual, auditory or haptic stimulation. The feedback may include personalized recommendations, activities, or connections to support the user's social well-being. In some embodiments, the user agency and capability augmentation system 100 may be further enhanced by incorporating therapist or physician guidance background material 108 or other context input in order to achieve a long-term mental health care objective.

### Enhancing Museum or Other Public or Novel Location Experience

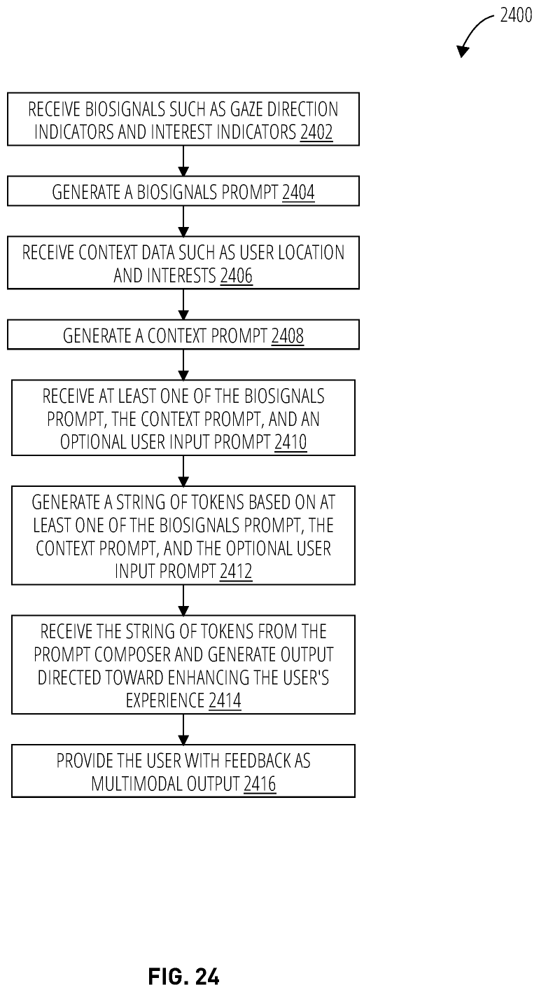

FIG. 24 illustrates an example routine 2400 for enhancing museum or other public or novel location experience. Although the example routine 2400 depicts a particular sequence of operations, the sequence may be altered without departing from the scope of the present disclosure. For example, some of the operations depicted may be performed in parallel or in a different sequence that does not materially affect the function of the routine 2400. In other examples, different components of an example device or system that implements the routine 2400 may perform functions at substantially the same time or in a specific sequence.

According to some examples, the method includes receiving biosignals such as gaze direction indicators and interest indicators at block 2402. For example, the biosignals subsystem 200 illustrated in FIG. 2 may receive biosignals such as gaze direction indicators and interest indicators. The biosignals 106 indicative of gaze direction may include camera data capturing the user's gaze. User 102 interest may be interpreted from heart rate and EEG signals.

According to some examples, the method includes generating a biosignals prompt at block 2404. For example, the biosignals subsystem 200 illustrated in FIG. 2 may generate a biosignals prompt. The biosignals prompt 136 may be generated by tokenizing the received biosignals 106.

According to some examples, the method includes receiving context data such as user location and interests at block 2406. For example, the context subsystem 300 illustrated in FIG. 3 may receive context data such as user location and interests. Sensor data 110 may be used to detect characteristics of the user's surroundings, such as objects, signage, sounds, etc. Other device data 112 may give details on the user's geographic location, from a smart phone GPS app, for example. Background material 108 available to the context subsystem 300 may include the user's language history, search history, and interests.

According to some examples, the method includes generating a context prompt at block 2408. For example, the context subsystem 300 illustrated in FIG. 3 may generate a context prompt. The context prompt 138 may be generated by tokenizing the received context data.

According to some examples, the method includes receiving at least one of the biosignals prompt, the context prompt, and an optional user input prompt at block 2410. For example, the prompt composer 116 illustrated in FIG. 1 may receive at least one of the biosignals prompt, the context prompt, and an optional user input prompt. The user input prompt 140 may include keywords or commands directly related to a user's real-time questions or interests.

According to some examples, the method includes generating a string of tokens based on at least one of the biosignals prompt, the context prompt, and the optional user input prompt at block 2412. For example, the prompt composer 116 illustrated in FIG. 1 may generate a string of tokens based on at least one of the biosignals prompt, the context prompt, and the optional user input prompt.

According to some examples, the method includes receiving the string of tokens from the prompt composer and generating output directed toward enhancing the user's experience at block 2414. For example, the GenAI 118 illustrated in FIG. 1 may receive the string of tokens from the prompt composer and generate output directed toward enhancing the user's experience.

According to some examples, the method includes providing the user with feedback as multimodal output at block 2416. For example, the GenAI 118 illustrated in FIG. 1 may provide the user with feedback as multimodal output. This feedback may be provided in the form of visual, auditory or haptic stimulation. Such feedback may provide real-time descriptions, translations, or explanations of the exhibits or other surrounding features of interest, guiding the user 102 to areas of particular interest, and relating exhibits to their existing knowledge. The feedback in one embodiment may be provided as an AR overlay presented to the user 102 by the wearable computing and biosignal sensing device 104.

In a related embodiment, a user 102 may be learning a new skill and may request an AR system to provide supplementary explanations, links to related materials, or other useful information as the user 102 navigates through a new task or new activity. In some embodiments, the user agency and capability augmentation system 100 may further record biosignals 106 or other estimates of the user's comfort or comprehension of the material and may utilize this to provide feedback to the user 102 or others on user confidence/competence/comprehension of a novel task or environment. In some versions of this embodiment, the feedback may further be used to generate a user-specific training, instruction, or evaluation plan or curriculum.

### Improving Hospital Experience

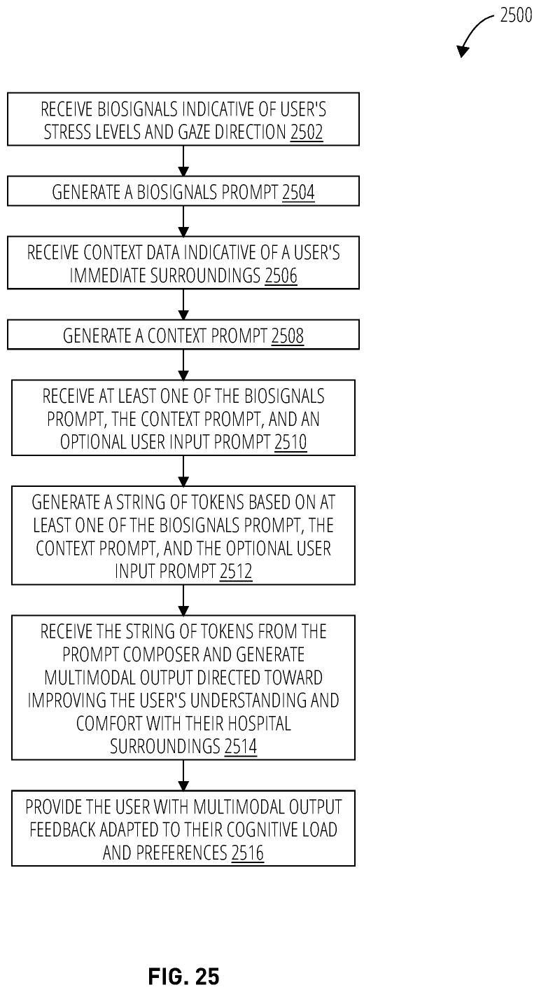

FIG. 25 illustrates an example routine 2500 for improving a user's hospital experience. Although the example routine 2500 depicts a particular sequence of operations, the sequence may be altered without departing from the scope of the present disclosure. For example, some of the operations depicted may be performed in parallel or in a different sequence that does not materially affect the function of the routine 2500. In other examples, different components of an example device or system that implements the routine 2500 may perform functions at substantially the same time or in a specific sequence.

According to some examples, the method includes receiving biosignals indicative of user's stress levels and gaze direction at block 2502. For example, the biosignals subsystem 200 illustrated in FIG. 2 may receive biosignals indicative of user's stress levels and gaze direction. Biosignals 106 may also be detected which indicate the user's cognitive load and curiosity or interest. Such biosignals 106 may include EEG data, heart and respiratory rate data, and gaze detection indicators from cameras or other sensors.

According to some examples, the method includes generating a biosignals prompt at block 2504. For example, the biosignals subsystem 200 illustrated in FIG. 2 may generate a biosignals prompt. The biosignals prompt 136 may be generated by tokenizing the received biosignals 106.

According to some examples, the method includes receiving context data indicative of a user's immediate surroundings at block 2506. For example, the context subsystem 300 illustrated in FIG. 3 may receive context data indicative of a user's immediate surroundings. Context data may include sensor data 110 in the form of images from cameras. Context data may also include background material 108 such as the user's appointments scheduled, the user's familiarity with the procedures they are undergoing and the equipment involved, or familiarity with medical procedures and equipment in general.

According to some examples, the method includes generating a context prompt at block 2508. For example, the context subsystem 300 illustrated in FIG. 3 may generate a context prompt. The context prompt 138 may be generated by tokenizing the received context data.

According to some examples, the method includes receiving at least one of the biosignals prompt, the context prompt, and an optional user input prompt at block 2510. For example, the prompt composer 116 illustrated in FIG. 1 may receive at least one of the biosignals prompt, the context prompt, and an optional user input prompt. The user input prompt 140 may include keywords or queries indicating a request for specific information, or details on equipment detected in their immediate field of view.

According to some examples, the method includes generating a string of tokens based on at least one of the biosignals prompt, the context prompt, and the optional user input prompt at block 2512. For example, the prompt composer 116 illustrated in FIG. 1 may generate a string of tokens based on at least one of the biosignals prompt, the context prompt, and the optional user input prompt.

According to some examples, the method includes receiving the string of tokens from the prompt composer and generating multimodal output directed toward improving the user's understanding and comfort with their hospital surroundings at block 2514. For example, the GenAI 118 illustrated in FIG. 1 may receive the string of tokens from the prompt composer and generate multimodal output directed toward improving the user's understanding and comfort with their hospital surroundings.

According to some examples, the method includes providing the user with multimodal output feedback adapted to their cognitive load and preferences at block 2516. For example, the GenAI 118 illustrated in FIG. 1 may provide the user with multimodal output feedback adapted to their cognitive load and preferences. This feedback may be provided in the form of visual, auditory or haptic stimulation. The feedback may be presented in an AR environment provided by the wearable computing and biosignal sensing device 104 or other AR device. The AR environment may provide labels for medical equipment as well as real-time information, guidance, and instructions. This feedback may be filtered and prioritized based on user 102 location and gaze direction, as well as estimated interest and arousal. In some embodiments, hospital staff may interact with the system by explicitly providing background material 108 to the context subsystem 300 in order to allow the user 102 to interact with relevant information in a self-guided and natural language mediated fashion.

### Improving Language Translation

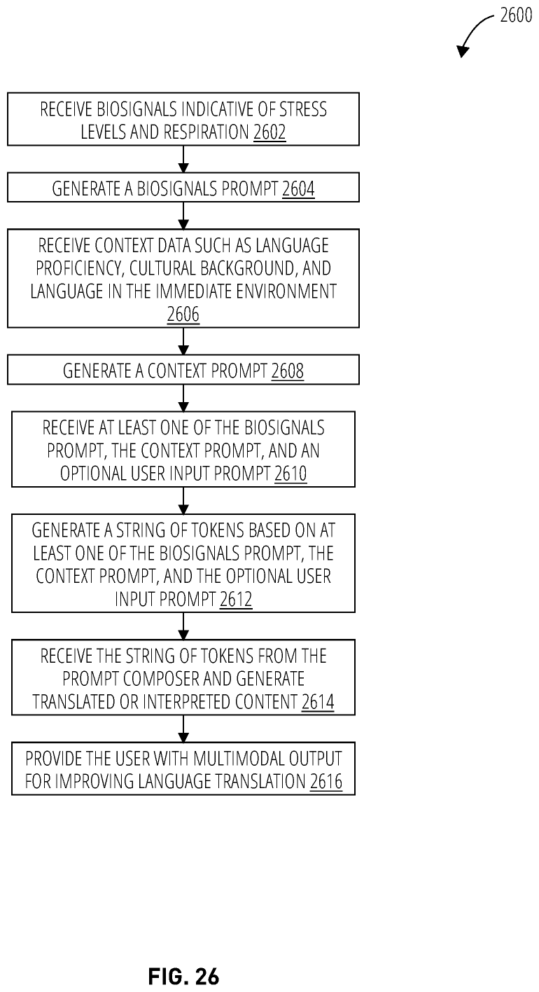

FIG. 26 illustrates an example routine 2600 for improving language translation. Although the example routine 2600 depicts a particular sequence of operations, the sequence may be altered without departing from the scope of the present disclosure. For example, some of the operations depicted may be performed in parallel or in a different sequence that does not materially affect the function of the routine 2600. In other examples, different components of an example device or system that implements the routine 2600 may perform functions at substantially the same time or in a specific sequence.

According to some examples, the method includes receiving biosignals indicative of stress levels and respiration at block 2602. For example, the biosignals subsystem 200 illustrated in FIG. 2 may receive biosignals indicative of stress levels and respiration. These biosignals 106 may be provided by the wearable computing and biosignal sensing device 104 or by additional biosensors 202 in the biosignals subsystem 200. Such biosignals 106 may include EEG data, heart and respiratory rate data, skin conductivity, etc.

According to some examples, the method includes generating a biosignals prompt at block 2604. For example, the biosignals subsystem 200 illustrated in FIG. 2 may generate a biosignals prompt. The biosignals prompt 136 may be generated by tokenizing the received biosignals 106.

According to some examples, the method includes receiving context data such as language proficiency, cultural background, and language in the immediate environment at block 2606. For example, the context subsystem 300 illustrated in FIG. 3 may receive context data such as language proficiency, cultural background, and language in the immediate environment. This context data may be provided as background material 108 and may be used to infer the user's translation needs. Context data may also include sensor data 110 such as audible spoken language detected by microphones, or video from cameras which may be analyzed for written text content.

According to some examples, the method includes generating a context prompt at block 2608. For example, the context subsystem 300 illustrated in FIG. 3 may generate a context prompt. The context prompt 138 may be generated by tokenizing the received context data.

According to some examples, the method includes receiving at least one of the biosignals prompt, the context prompt, and an optional user input prompt at block 2610. For example, the prompt composer 116 illustrated in FIG. 1 may receive at least one of the biosignals prompt, the context prompt, and an optional user input prompt. The user input prompt 140 may include keywords, queries, or commands related to spoken or written language in the user's environment desired speech output.

According to some examples, the method includes generating a string of tokens based on at least one of the biosignals prompt, the context prompt, and the optional user input prompt at block 2612. For example, the prompt composer 116 illustrated in FIG. 1 may generate a string of tokens based on at least one of the biosignals prompt, the context prompt, and the optional user input prompt.

According to some examples, the method includes receiving the string of tokens from the prompt composer and generating translated or interpreted content at block 2614. For example, the GenAI 118 illustrated in FIG. 1 may receive the string of tokens from the prompt composer and generate translated or interpreted content. The GenAI 118 may be a pre-trained natural language 118 configured to provide personalized language translation and interpretation services to the user 102.

According to some examples, the method includes providing the user with multimodal output for improving language translation at block 2616. For example, the GenAI 118 illustrated in FIG. 1 may provide the user with multimodal output for improving language translation. Translations and interpretations may be provided to the user 102 via visual stimuli of an AR/VR display, such as the wearable computing and biosignal sensing device 104, and/or by auditory stimuli.

### Improving Driving Safety

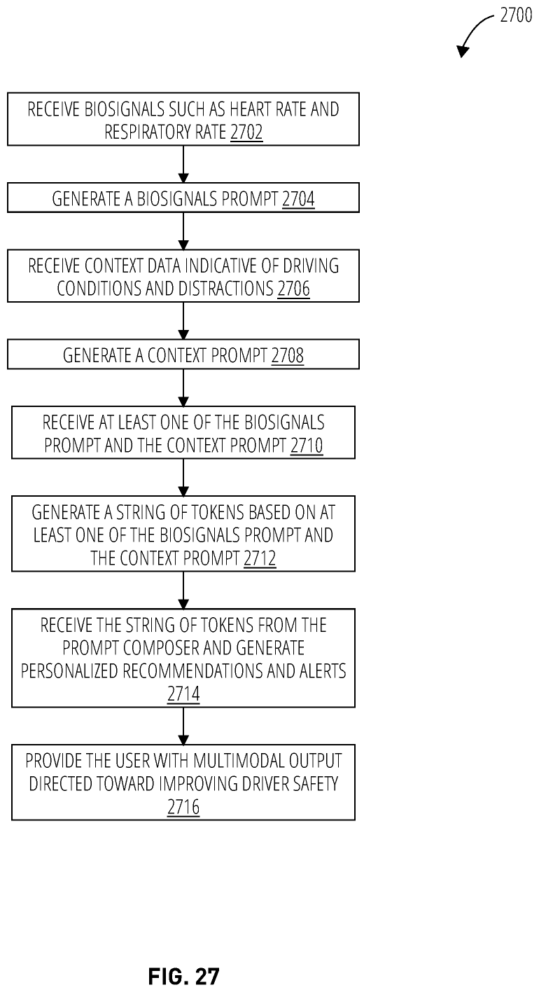

FIG. 27 illustrates an example routine 2700 for improving driving safety or controlling/piloting other vehicles or aircraft. Although the example routine 2700 depicts a particular sequence of operations, the sequence may be altered without departing from the scope of the present disclosure. For example, some of the operations depicted may be performed in parallel or in a different sequence that does not materially affect the function of the routine 2700. In other examples, different components of an example device or system that implements the routine 2700 may perform functions at substantially the same time or in a specific sequence.

According to some examples, the method includes receiving biosignals such as heart rate and respiratory rate at block 2702. For example, the biosignals subsystem 200 illustrated in FIG. 2 may receive biosignals such as heart rate and respiratory rate. Additional signals indicative of user distraction may be detected by the wearable computing and biosignal sensing device 104 or additional biosensors 202, including EEG data.

According to some examples, the method includes generating a biosignals prompt at block 2704. For example, the biosignals subsystem 200 illustrated in FIG. 2 may generate a biosignals prompt. The biosignals prompt 136 may be generated by tokenizing the received biosignals 106.

According to some examples, the method includes receiving context data indicative of driving conditions and distractions at block 2706. For example, the context subsystem 300 illustrated in FIG. 3 may receive context data indicative of driving conditions and distractions. Sensor data 110 such as microphone output indicating ambient noise and camera output indicating the driver's visual surroundings may be used to infer driving conditions and potential distractions.

According to some examples, the method includes generating a context prompt at block 2708. For example, the context subsystem 300 illustrated in FIG. 3 may generate a context prompt. The context prompt 138 may be generated by tokenizing the received context data.

According to some examples, the method includes receiving at least one of the biosignals prompt and the context prompt at block 2710. For example, the prompt composer 116 illustrated in FIG. 1 may receive at least one of the biosignals prompt and the context prompt.

According to some examples, the method includes generating a string of tokens based on at least one of the biosignals prompt and the context prompt at block 2712. For example, the prompt composer 116 illustrated in FIG. 1 may generate a string of tokens based on at least one of the biosignals prompt and the context prompt.

According to some examples, the method includes receiving the string of tokens from the prompt composer and generating personalized recommendations and alerts at block 2714. For example, the GenAI 118 illustrated in FIG. 1 may receive the string of tokens from the prompt composer and generate personalized recommendations and alerts. The GenAI 118 may be a pre-trained natural language GenAI 118 configured to provide personalized driving safety recommendations and alerts to the user 102 when prompted.

According to some examples, the method includes providing the user with multimodal output directed toward improving driver safety at block 2716. For example, the GenAI 118 illustrated in FIG. 1 may provide the user with multimodal output directed toward improving driver safety or improving control or operational skills. The multimodal output may be provided to the user as messages and alerts delivered via visual stimuli of an AR/VR display, by auditory stimuli or by haptic feedback through vehicle instrumentation. In some instances, the system will identify driver specific deficiencies and communicate these to a driver support system such as an autonomous vehicle control subsystem. In other embodiments, the system will identify specific areas where the user needs to undergo additional training. In one embodiment, the system may detect when a driver is distracted and may use multimodal output such as haptic, audio, and visual feedback to recapture their attention in a way that does not result in a startle response to help get them back on task. Contextual analysis of the total driving scene as well as the user's mental state (inferred via biosignals and other information integration such as steering wheel input forces) may be used to determine the strength or intensity of an alert designed to recapture a driver's awareness.

### Dialog System Using Biosensing to Drive Responses

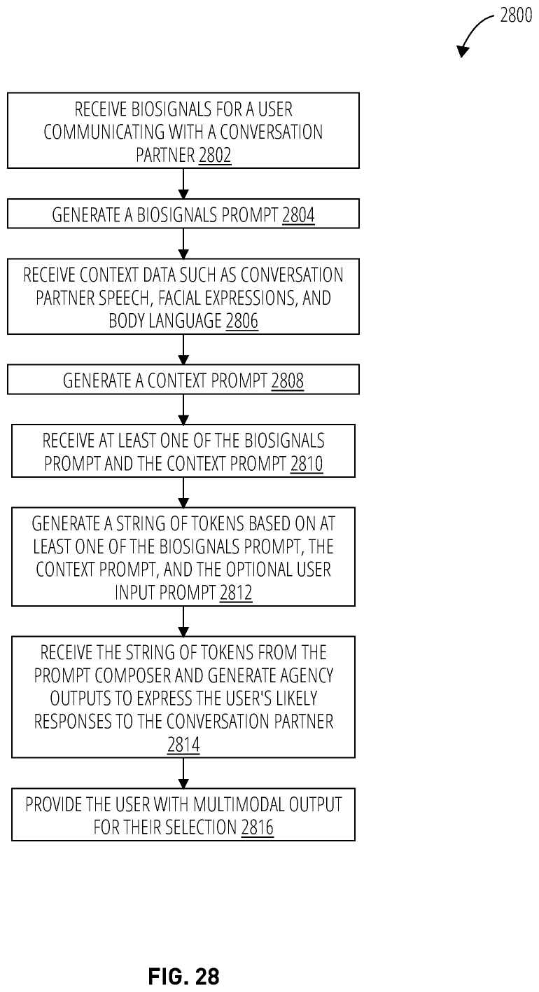

FIG. 28 illustrates an example routine 2800 for dialog system using biosensing to drive responses. Although the example routine 2800 depicts a particular sequence of operations, the sequence may be altered without departing from the scope of the present disclosure. For example, some of the operations depicted may be performed in parallel or in a different sequence that does not materially affect the function of the routine 2800. In other examples, different components of an example device or system that implements the routine 2800 may perform functions at substantially the same time or in a specific sequence.

According to some examples, the method includes receiving biosignals for a user communicating with a conversation partner at block 2802. For example, the biosignals subsystem 200 illustrated in FIG. 2 may receive biosignals for a user communicating with a conversation partner. The biosignals 106 may be provided by a wearable computing and biosignal sensing device 104 worn by the user 102 or additional biosensors 202.

According to some examples, the method includes generating a biosignals prompt at block 2804. For example, the biosignals subsystem 200 illustrated in FIG. 2 may generate a biosignals prompt. The biosignals prompt 136 may be generated by tokenizing the received biosignals 106 to indicate the user's physiological and cognitive responses.

According to some examples, the method includes receiving context data such as conversation partner speech, facial expressions, and body language at block 2806. For example, the context subsystem 300 illustrated in FIG. 3 may receive context data such as conversation partner speech, facial expressions, and body language.

According to some examples, the method includes generating a context prompt at block 2808. For example, the context subsystem 300 illustrated in FIG. 3 may generate a context prompt. The context prompt 138 may be generated by tokenizing the audio and video capturing the conversation partner's communication.

According to some examples, the method includes receiving at least one of the biosignals prompt and the context prompt at block 2810. For example, the prompt composer 116 illustrated in FIG. 1 may receive at least one of the biosignals prompt and the context prompt. In this embodiment, no user input prompt 140 may be needed to drive predictions.

According to some examples, the method includes generating a string of tokens based on at least one of the biosignals prompt, the context prompt, and the optional user input prompt at block 2812. For example, the prompt composer 116 illustrated in FIG. 1 may generate a string of tokens based on at least one of the biosignals prompt, the context prompt, and the optional user input prompt.

According to some examples, the method includes receiving the string of tokens from the prompt composer and generating agency outputs to express the user's likely responses to the conversation partner at block 2814. For example, the GenAI 118 illustrated in FIG. 1 may receive the string of tokens from the prompt composer and generate agency outputs to express the user's likely responses to the conversation partner.

According to some examples, the method includes providing the user with multimodal output for their selection at block 2816. For example, the GenAI 118 illustrated in FIG. 1 may provide the user with multimodal output for their selection. The user 102 may then select appropriate responses and have them communicated to their conversation partner. In one embodiment, “nodding head and listening with interest” may be an output from the GenAI 118, which may then be used to drive a robotic system, an avatar representing the user 102, or simply an emoji response that is rendered back to the conversation partner. In another embodiment, a user's physical state may be used to prioritize utterances 124 that address their immediate needs or activities, such as requesting assistance or exposing health monitor information to the user 102 when exercise activities are detected.

### Using Biosignals to Infer a Tone or Style for Model Output

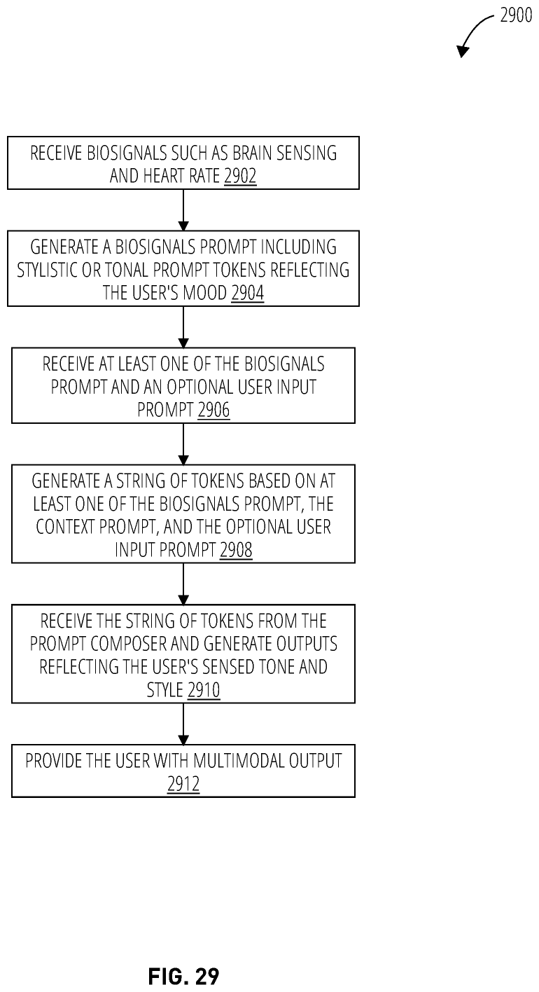

FIG. 29 illustrates an example routine 2900 for using biosignals to infer a tone or style for model output. Although the example routine 2900 depicts a particular sequence of operations, the sequence may be altered without departing from the scope of the present disclosure. For example, some of the operations depicted may be performed in parallel or in a different sequence that does not materially affect the function of the routine 2900. In other examples, different components of an example device or system that implements the routine 2900 may perform functions at substantially the same time or in a specific sequence.

According to some examples, the method includes receiving biosignals such as brain sensing and heart rate at block 2902. For example, the biosignals subsystem 200 illustrated in FIG. 2 may receive biosignals such as brain sensing and heart rate. Biosignals 106 may be detected from the wearable computing and biosignal sensing device 104 or additional biosensors 202. Biosignals 106 may include EEG data in one embodiment. The user agency and capability augmentation system 100 may use brain sensing data and/or heart rate data to estimate the user's level of arousal or engagement with the context.

According to some examples, the method includes generating a biosignals prompt including stylistic or tonal prompt tokens reflecting the user's mood at block 2904. For example, the biosignals subsystem 200 illustrated in FIG. 2 may generate a biosignals prompt including stylistic or tonal prompt tokens reflecting the user's mood. When arousal is high, the biosignals subsystem 200 may generate more emphatic or excited tokens, such as “excited, happy, engaged”. When arousal is low, the biosignals subsystem 200 tokenizer may generate tokens with less interest or excitement, such as “bored, disinterested”.

According to some examples, the method includes receiving at least one of the biosignals prompt and an optional user input prompt at block 2906. For example, the prompt composer 116 illustrated in FIG. 1 may receive at least one of the biosignals prompt and an optional user input prompt. The user input prompt 140 may include keywords or commands from the user 102, such as selecting as the user input prompt 140 an emoji to alter the output's persona, tone, or “mood.”.

According to some examples, the method includes generating a string of tokens based on at least one of the biosignals prompt, the context prompt, and the optional user input prompt at block 2908. For example, the prompt composer 116 illustrated in FIG. 1 may generate a string of tokens based on at least one of the biosignals prompt, the context prompt, and the optional user input prompt.

According to some examples, the method includes receiving the string of tokens from the prompt composer and generating outputs reflecting the user's sensed tone and style at block 2910. For example, the GenAI 118 illustrated in FIG. 1 may receive the string of tokens from the prompt composer and generate outputs reflecting the user's sensed tone and style.

According to some examples, the method includes providing the user with multimodal output at block 2912. For example, the GenAI 118 illustrated in FIG. 1 may provide the user with multimodal output.

### Sensing a State of a Controllable or Semi-Autonomous Entity

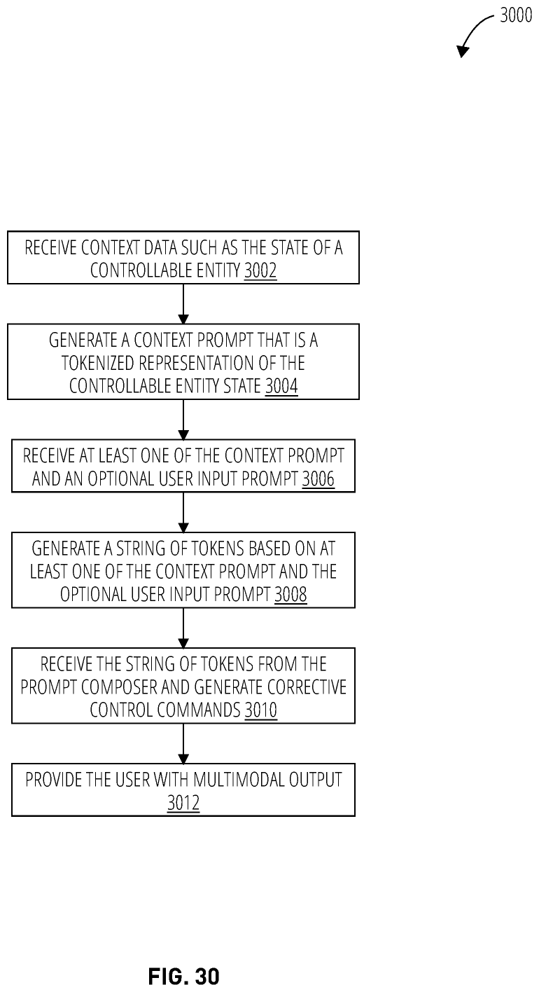

FIG. 30 illustrates an example routine 3000 for sensing a state of a controllable or semi-autonomous entity. Although the example routine 3000 depicts a particular sequence of operations, the sequence may be altered without departing from the scope of the present disclosure. For example, some of the operations depicted may be performed in parallel or in a different sequence that does not materially affect the function of the routine 3000. In other examples, different components of an example device or system that implements the routine 3000 may perform functions at substantially the same time or in a specific sequence.

According to some examples, the method includes receiving context data such as the state of a controllable or semi-autonomous entity at block 3002. For example, the context subsystem 300 illustrated in FIG. 3 may receive context data such as the state of the controllable or semi-autonomous entity. The state of the entity, such as a vehicle, assembly line, or robotic system, may be provided as sensor data 110 or other device data 112 from sensors or state machines integrated into the entity.

According to some examples, the method includes generating a context prompt that is a tokenized representation of the controllable or semi-autonomous entity state at block 3004. For example, the context subsystem 300 illustrated in FIG. 3 may generate a context prompt that is a tokenized representation of the controllable or semi-autonomous entity state.

According to some examples, the method includes receiving at least one of the context prompt and an optional user input prompt at block 3006. For example, the prompt composer 116 illustrated in FIG. 1 may receive at least one of the context prompt and an optional user input prompt. The user input prompt 140 may indicate the.

According to some examples, the method includes generating a string of tokens based on at least one of the context prompt and the optional user input prompt at block 3008. For example, the prompt composer 116 illustrated in FIG. 1 may generate a string of tokens based on at least one of the context prompt and the optional user input prompt.

According to some examples, the method includes receiving the string of tokens from the prompt composer and generating corrective control commands at block 3010. For example, the GenAI 118 illustrated in FIG. 1 may receive the string of tokens from the prompt composer and generate corrective control commands. The corrective control commands may be directed toward modifying the state of the controllable or semi-autonomous entity.

According to some examples, the method includes providing the user with multimodal output at block 3012. For example, the GenAI 118 illustrated in FIG. 1 may provide the user with multimodal output. In one exemplary embodiment, the context subsystem 300 may serve assembly line robots by detecting anomalies in the visual field that indicate changes in orientation of items in the assembly line.

Background is the User's Collected Works in a Specific Topic Area

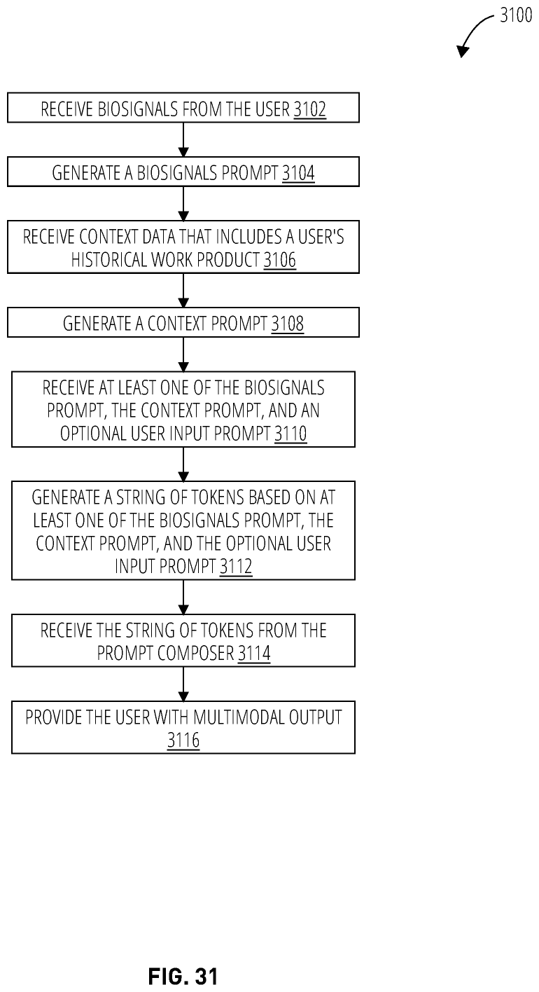

FIG. 31 illustrates an example routine 3100 when the background material 108 is the user's collected works in a specific topic area. Although the example routine 3100 depicts a particular sequence of operations, the sequence may be altered without departing from the scope of the present disclosure. For example, some of the operations depicted may be performed in parallel or in a different sequence that does not materially affect the function of the routine 3100. In other examples, different components of an example device or system that implements the routine 3100 may perform functions at substantially the same time or in a specific sequence.

According to some examples, the method includes receiving biosignals from the user at block 3102. For example, the biosignals subsystem 200 illustrated in FIG. 2 may receive biosignals from the user. Biosignals 106 may be detected from the wearable computing and biosignal sensing device 104 or additional biosensors 202.

According to some examples, the method includes generating a biosignals prompt at block 3104. For example, the biosignals subsystem 200 illustrated in FIG. 2 may generate a biosignals prompt. The biosignals prompt 136 may be generated by tokenizing the received biosignals 106.

According to some examples, the method includes receiving context data that includes a user's historical work product at block 3106. For example, the context subsystem 300 illustrated in FIG. 3 may receive context data that includes a user's historical work product. The context subsystem 300 may receive background material 108 that a user's historical work product, such as a collection of documents, images, or other media that represent the user's collected works in a specific topic area. For example, the user 102 might be an expert in a specific topic such as eighteenth-century literature or electrical engineering. The user 102 may have written articles, curricula, videos, etc. These may be incorporated into the historical work product and summarized or tokenized as appropriate.

According to some examples, the method includes generating a context prompt at block 3108. For example, the context subsystem 300 illustrated in FIG. 3 may generate a context prompt. The context prompt 138 may be generated by tokenizing the received context data. The background material 108 (historical information) may be incorporated into the context token stream and provided as background to the prompt composer 116.

According to some examples, the method includes receiving at least one of the biosignals prompt, the context prompt, and an optional user input prompt at block 3110. For example, the prompt composer 116 illustrated in FIG. 1 may receive at least one of the biosignals prompt, the context prompt, and an optional user input prompt.

According to some examples, the method includes generating a string of tokens based on at least one of the biosignals prompt, the context prompt, and the optional user input prompt at block 3112. For example, the prompt composer 116 illustrated in FIG. 1 may generate a string of tokens based on at least one of the biosignals prompt, the context prompt, and the optional user input prompt. In this configuration, the biosignals prompt 136, context prompt 138, and user input prompt 140 may be used to construct a prompt that specifically relates to the historical work product.

According to some examples, the method includes receiving the string of tokens from the prompt composer at block 3114. For example, the GenAI 118 illustrated in FIG. 1 may receive the string of tokens from the prompt composer. In some embodiments, the amount of background material 108 in the form of historical work product may be substantial and may alternatively need the GenAI 118 itself be retrained with this additional information.

According to some examples, the method includes providing the user with multimodal output at block 3116. For example, the GenAI 118 illustrated in FIG. 1 may provide the user with multimodal output. In one use case, this may cause output text from the GenAI 118 to be in the style of the background material 108 or framed in the technical jargon of a specific technical discipline.

Pretraining the GenAI with Historical User Works for Improved Response to Compositional Prompts

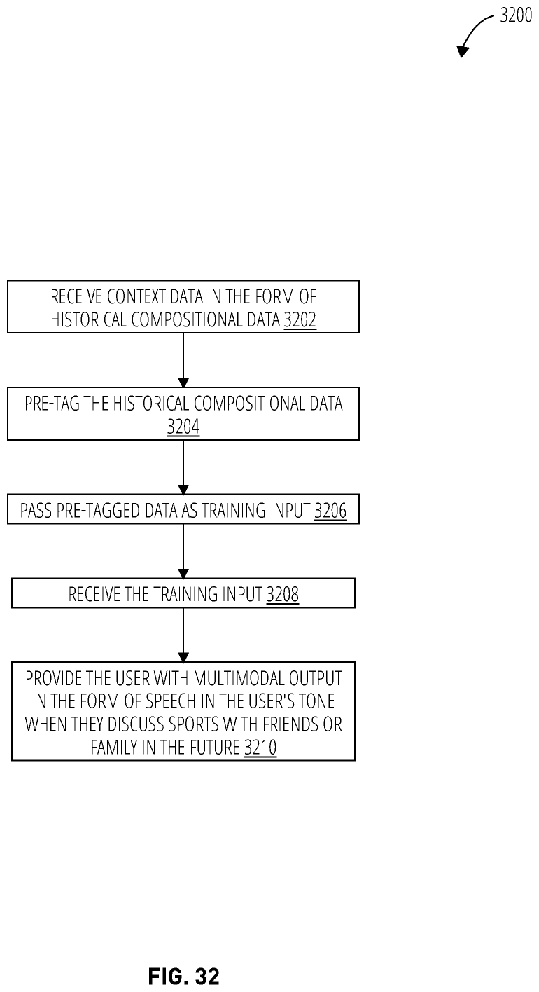

FIG. 32 illustrates an example routine 3200 for pretraining the GenAI with historical user works for improved response to compositional prompts. Although the example routine 3200 depicts a particular sequence of operations, the sequence may be altered without departing from the scope of the present disclosure. For example, some of the operations depicted may be performed in parallel or in a different sequence that does not materially affect the function of the routine 3200. In other examples, different components of an example device or system that implements the routine 3200 may perform functions at substantially the same time or in a specific sequence.

According to some examples, the method includes receiving context data in the form of historical compositional data at block 3202. For example, the context subsystem 300 illustrated in FIG. 3 may receive context data in the form of historical compositional data. In one embodiment, the background material 108 (historical compositional data) may be pre-tagged with relevant semantic context tokens prior to any utterances being generated.

According to some examples, the method includes pre-tagging the historical compositional data at block 3204. For example, the prompt composer 116 illustrated in FIG. 1 may pre-tag the historical compositional data. For example, a user 102 may import their chat history, which may be tagged not only with the conversation partner but also with the type of conversation, such as an informal one about sports. In one embodiment, the context subsystem 300 may perform the pre-tagging.

According to some examples, the method includes passing pre-tagged data as training input at block 3206. For example, the prompt composer 116 illustrated in FIG. 1 may pass pre-tagged data as training input.

According to some examples, the method includes receiving the training input at block 3208. For example, the GenAI 118 illustrated in FIG. 1 may receive the training input. Using the training input, the GenAI 118 may be tuned so that it is capable of generating output that contains more user-specific knowledge, as well as tone appropriate to the target output format.

According to some examples, the method includes providing the user with multimodal output in the form of speech in the user's tone when they discuss sports with friends or family in the future at block 3210. For example, the GenAI 118 illustrated in FIG. 1 may provide the user with multimodal output in the form of speech in the user's tone when they discuss sports with friends or family in the future.

### Background is a Specific Set of Instructions for a User Task

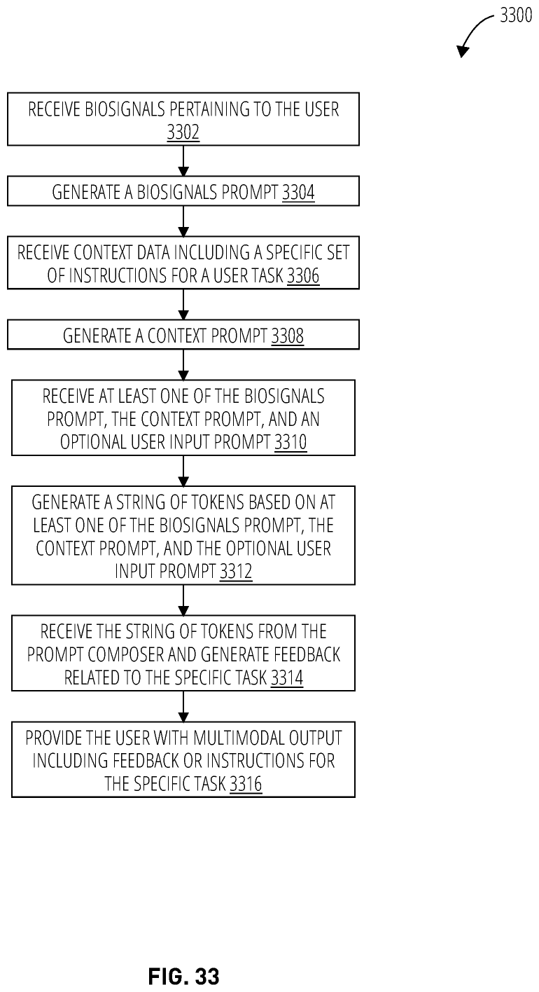

FIG. 33 illustrates an example routine 3300 for when the background material 108 is a specific set of instructions for a user task. Although the example routine 3300 depicts a particular sequence of operations, the sequence may be altered without departing from the scope of the present disclosure. For example, some of the operations depicted may be performed in parallel or in a different sequence that does not materially affect the function of the routine 3300. In other examples, different components of an example device or system that implements the routine 3300 may perform functions at substantially the same time or in a specific sequence.

According to some examples, the method includes receiving biosignals pertaining to the user at block 3302. For example, the biosignals subsystem 200 illustrated in FIG. 2 may receive biosignals pertaining to the user. The biosignals subsystem 200 may estimate the user's body configuration as well as their level of mental workload, fatigue, and frustration with the task.

According to some examples, the method includes generating a biosignals prompt at block 3304. For example, the biosignals subsystem 200 illustrated in FIG. 2 may generate a biosignals prompt. The biosignals prompt 136 may be generated by tokenizing the received biosignals 106.

According to some examples, the method includes receiving context data including a specific set of instructions for a user task at block 3306. For example, the context subsystem 300 illustrated in FIG. 3 may receive context data including a specific set of instructions for a user task. The specific set of instructions may be provided as background material 108 and may be related to a task that the user 102 needs to perform or will soon perform. The context subsystem 300 may also receive sensor data 110 and/or other device data 112 allowing it to estimate the appropriate state of the target of the task and may detail the steps needed to accomplish the task with chain-of-thought reasoning. The user 102 may review the steps and approve their execution in some embodiments, such as in computer programming. The task may be in nearly any domain, including cooking, mechanical repair, craft-making, or computer programming.

According to some examples, the method includes generating a context prompt at block 3308. For example, the context subsystem 300 illustrated in FIG. 3 may generate a context prompt. The context prompt 138 may be generated by tokenizing the received context data.

According to some examples, the method includes receiving at least one of the biosignals prompt, the context prompt, and an optional user input prompt at block 3310. For example, the prompt composer 116 illustrated in FIG. 1 may receive at least one of the biosignals prompt, the context prompt, and an optional user input prompt. The user input prompt 140 may include user 102 instructions for pacing the task execution.

According to some examples, the method includes generating a string of tokens based on at least one of the biosignals prompt, the context prompt, and the optional user input prompt at block 3312. For example, the prompt composer 116 illustrated in FIG. 1 may generate a string of tokens based on at least one of the biosignals prompt, the context prompt, and the optional user input prompt. The prompt composer 116 may create a prompt that asks the GenAI 118 to generate instructions for the next step in the process and/or for the user agency and capability augmentation system 100 to provide feedback on the user's performance of the task.

According to some examples, the method includes receiving the string of tokens from the prompt composer and generating feedback related to the specific task at block 3314. For example, the GenAI 118 illustrated in FIG. 1 may receive the string of tokens from the prompt composer and generate feedback related to the specific task.

According to some examples, the method includes providing the user with multimodal output including feedback or instructions for the specific task at block 3316. For example, the GenAI 118 illustrated in FIG. 1 may provide the user with multimodal output including feedback or instructions for the specific task. In some embodiments, this feedback may include coaching to improve user 102 confidence or reduce anxiety. The instructions or coaching feedback may be provided to the user 102 as visual or auditory stimuli using an appropriate rendering device such as a pair of AR/VR glasses, a headphone, or the wearable computing and biosignal sensing device 104.

Using Context to Estimate the Difference between Observed and Expected State

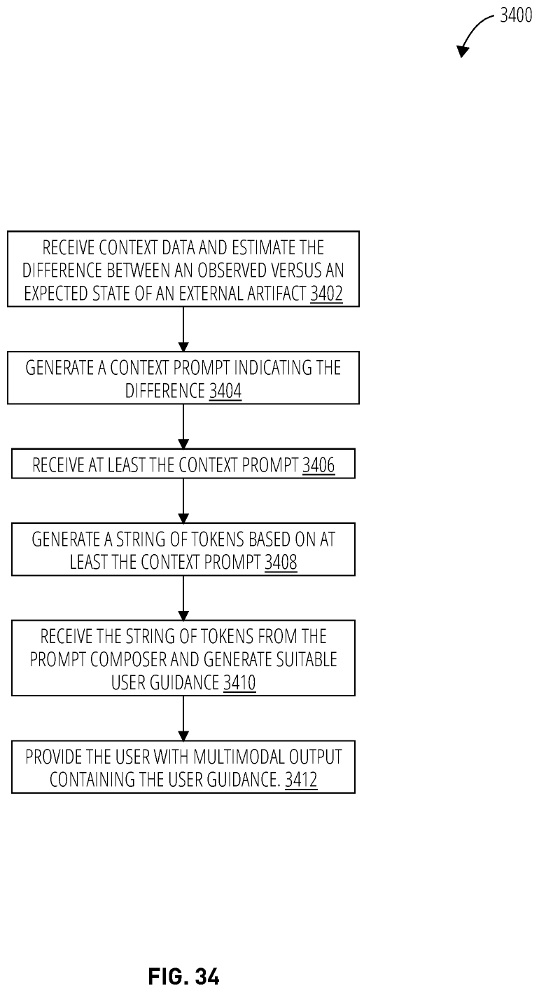

FIG. 34 illustrates an example routine 3400 for context estimate is the difference between observed and expected. Although the example routine 3400 depicts a particular sequence of operations, the sequence may be altered without departing from the scope of the present disclosure. For example, some of the operations depicted may be performed in parallel or in a different sequence that does not materially affect the function of the routine 3400. In other examples, different components of an example device or system that implements the routine 3400 may perform functions at substantially the same time or in a specific sequence.

According to some examples, the method includes receiving context data and estimating the difference between an observed versus an expected state of an external artifact at block 3402. For example, the context subsystem 300 illustrated in FIG. 3 may receive context data and estimate the difference between an observed versus an expected state of an external artifact. The context subsystem 300 may use available sensor data 110 and background material 108 to estimate the difference between the observed state of the external artifact and a desired or expected state.

According to some examples, the method includes generating a context prompt indicating the difference at block 3404. For example, the context subsystem 300 illustrated in FIG. 3 may generate a context prompt indicating the difference. The context prompt 138 may be generated by tokenizing the received context data. The context subsystem 300 may drive the prompt composer 116 to ensure that the user 102 has the correct tools for the job at hand.

According to some examples, the method includes receiving at least the context prompt at block 3406. For example, the prompt composer 116 illustrated in FIG. 1 may receive at least the context prompt.

According to some examples, the method includes generating a string of tokens based on at least the context prompt at block 3408. For example, the prompt composer 116 illustrated in FIG. 1 may generate a string of tokens based on at least the context prompt. In another use case, the context subsystem 300 may use external sensors to identify a potentially hazardous situation such as collapsed roadway or pathway that a user 102 is walking along and drive the prompt composer to prompt the GenAI 118 for suitable warnings and correctives.

According to some examples, the method includes receiving the string of tokens from the prompt composer and generating suitable user guidance at block 3410. For example, the GenAI 118 illustrated in FIG. 1 may receive the string of tokens from the prompt composer and generate suitable user guidance.

According to some examples, the method includes providing the user with multimodal output containing the user guidance at block 3412. For example, the GenAI 118 illustrated in FIG. 1 may provide the user with multimodal output containing the user guidance. In one embodiment, the user guidance may include overlay text displayed in an AR environment available via the wearable computing and biosignal sensing device 104, or audible utterances 124, such as “you'll need a screwdriver with a T6 bit to complete this repair”. In one embodiment, the context discrepancy may be used to provide feedback to the user 102 which is then verified using the biosignals subsystem 200 (e.g., the user 102 stops or changes course, and this is detected by the wearable computing and biosignal sensing device 104 or additional biosensors 202).

Background Includes Labels Associated with Sensor Data

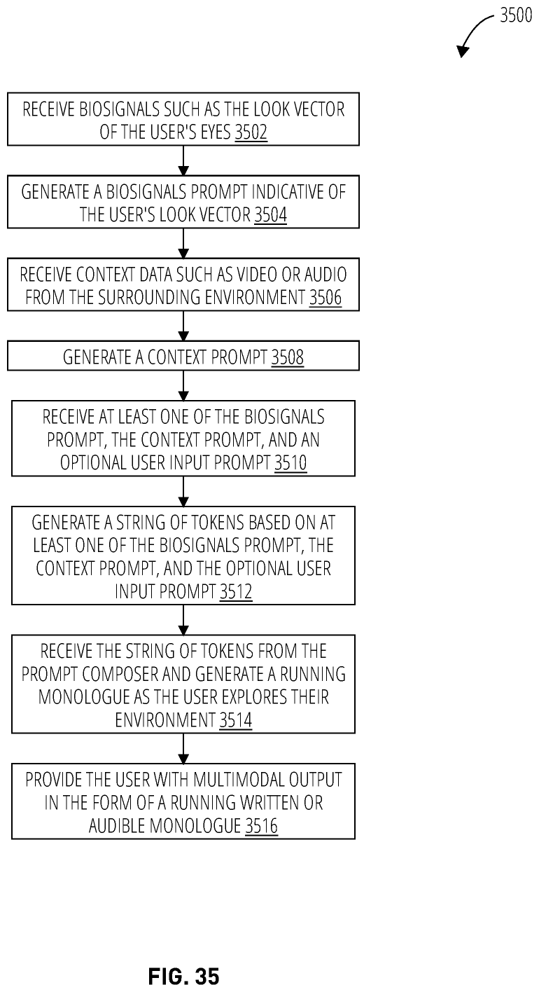

FIG. 35 illustrates an example routine 3500 when background includes labels associated with sensor data. Although the example routine 3500 depicts a particular sequence of operations, the sequence may be altered without departing from the scope of the present disclosure. For example, some of the operations depicted may be performed in parallel or in a different sequence that does not materially affect the function of the routine 3500. In other examples, different components of an example device or system that implements the routine 3500 may perform functions at substantially the same time or in a specific sequence.

According to some examples, the method includes receiving biosignals such as the look vector of the user's eyes at block 3502. For example, the biosignals subsystem 200 illustrated in FIG. 2 may receive biosignals such as the look vector of the user's eyes.

According to some examples, the method includes generating a biosignals prompt indicative of the user's look vector at block 3504. For example, the biosignals subsystem 200 illustrated in FIG. 2 may generate a biosignals prompt indicative of the user's look vector. The biosignals prompt 136 may be generated by tokenizing the received biosignals 106.

According to some examples, the method includes receiving context data such as video or audio from the surrounding environment at block 3506. For example, the context subsystem 300 illustrated in FIG. 3 may receive context data such as video or audio from the surrounding environment. In addition to sensor data 110 such as video and audio, the biosignals subsystem 200 may receive background material 108 in the form of informational labels that may be associated with elements in the environment detected through the sensor data 110.

According to some examples, the method includes generating a context prompt at block 3508. For example, the context subsystem 300 illustrated in FIG. 3 may generate a context prompt. The context prompt 138 may be generated by tokenizing the received context data. In one embodiment, the user 102 may be in a museum, and the context subsystem 300 may generate a prompt that classifies a specific artifact detected by video sensor as with the information available as background material 108.

According to some examples, the method includes receiving at least one of the biosignals prompt, the context prompt, and an optional user input prompt at block 3510. For example, the prompt composer 116 illustrated in FIG. 1 may receive at least one of the biosignals prompt, the context prompt, and an optional user input prompt. The user input prompt 140 may include keywords or queries related to information about the user's surroundings that is desired by the user 102.

According to some examples, the method includes generating a string of tokens based on at least one of the biosignals prompt, the context prompt, and the optional user input prompt at block 3512. For example, the prompt composer 116 illustrated in FIG. 1 may generate a string of tokens based on at least one of the biosignals prompt, the context prompt, and the optional user input prompt.

According to some examples, the method includes receiving the string of tokens from the prompt composer and generating a running monologue as the user explores their environment at block 3514. For example, the GenAI 118 illustrated in FIG. 1 may receive the string of tokens from the prompt composer and generate a running monologue as the user explores their environment. For example, the detected objects that are determined by biosignals 106 as intersecting the user's look vector and their informational labels may be used to provide contextually relevant commentary on an environment or artifact in the environment.

According to some examples, the method includes providing the user with multimodal output in the form of a running written or audible monologue at block 3516. For example, the GenAI 118 illustrated in FIG. 1 may provide the user with multimodal output in the form of a running written or audible monologue. In another embodiment, the multimodal output may provide the user with context-specific options based on the biosignals data. The user agency and capability augmentation system 100 may in one embodiment generate a running monologue as the user 102 explores a museum environment. Another embodiment may be expressive, wherein a forward-facing camera or other environment sensor may detect a person, object, or activity that may enrich potential GenAI 118 output. In one exemplary embodiment, a vision impaired user 102 may receive a running monologue of key classified elements in their surrounding environment in support of their navigation or other actions within an unfamiliar location. In a related embodiment, the user may further receive instructions or options that assist with environmental interactions.

Biosignal Tokenizer Includes Information about Desired vs. Observed User Body State

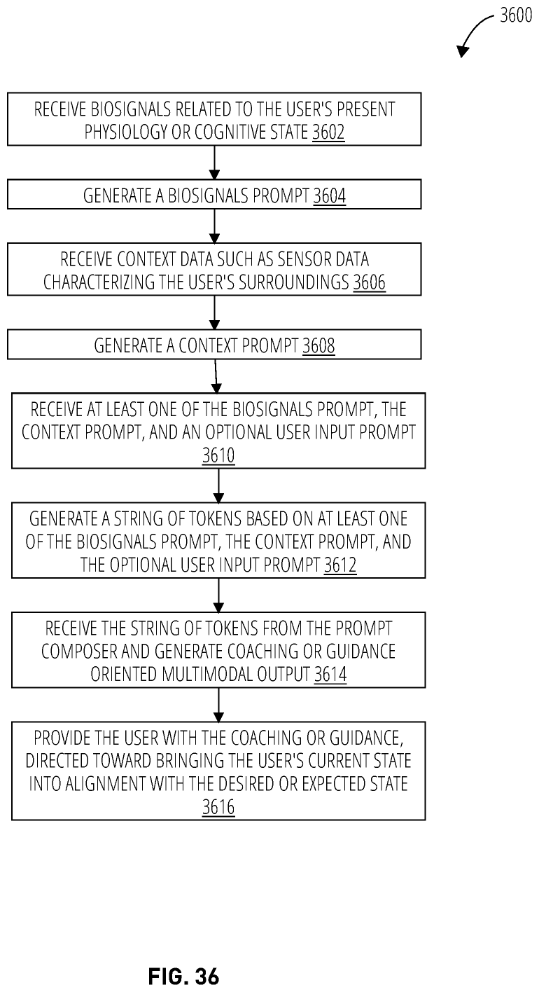

FIG. 36 illustrates an example routine 3600 where the biosignal tokenizer includes information about desired versus observed user body state. Although the example routine 3600 depicts a particular sequence of operations, the sequence may be altered without departing from the scope of the present disclosure. For example, some of the operations depicted may be performed in parallel or in a different sequence that does not materially affect the function of the routine 3600. In other examples, different components of an example device or system that implements the routine 3600 may perform functions at substantially the same time or in a specific sequence.

According to some examples, the method includes receiving biosignals related to the user's present physiology or cognitive state at block 3602. For example, the biosignals subsystem 200 illustrated in FIG. 2 may receive biosignals related to the user's present physiology or cognitive state.

According to some examples, the method includes generating a biosignals prompt at block 3604. For example, the biosignals subsystem 200 illustrated in FIG. 2 may generate a biosignals prompt. The biosignals prompt 136 may be generated by tokenizing the received biosignals 106. The biosignals prompt 136 may indicate a difference between the user's observed state and a desired or expected state.

According to some examples, the method includes receiving context data such as sensor data characterizing the user's surroundings at block 3606. For example, the context subsystem 300 illustrated in FIG. 3 may receive context data such as sensor data characterizing the user's surroundings. Sensor data 110 may include camera output showing features of the user's surroundings and the objects the user is interacting with.

According to some examples, the method includes generating a context prompt at block 3608. For example, the context subsystem 300 illustrated in FIG. 3 may generate a context prompt. The context prompt 138 may be generated by tokenizing the received context data.

According to some examples, the method includes receiving at least one of the biosignals prompt, the context prompt, and an optional user input prompt at block 3610. For example, the prompt composer 116 illustrated in FIG. 1 may receive at least one of the biosignals prompt, the context prompt, and an optional user input prompt. The user input prompt 140 may include explicit direction from the user regarding their desired or expected physiological or cognitive state.

According to some examples, the method includes generating a string of tokens based on at least one of the biosignals prompt, the context prompt, and the optional user input prompt at block 3612. For example, the prompt composer 116 illustrated in FIG. 1 may generate a string of tokens based on at least one of the biosignals prompt, the context prompt, and the optional user input prompt.

According to some examples, the method includes receiving the string of tokens from the prompt composer and generating coaching or guidance oriented multimodal output at block 3614. For example, the GenAI 118 illustrated in FIG. 1 may receive the string of tokens from the prompt composer and generate coaching or guidance oriented multimodal output.

According to some examples, the method includes providing the user with the coaching or guidance, directed toward bringing the user's current state into alignment with the desired or expected state at block 3616. For example, the GenAI 118 illustrated in FIG. 1 may provide the user with the coaching or guidance directed toward bringing the user's current state into alignment with the desired or expected state. In a use case, a user's blood sugar may be higher than expected and the user 102 may be in the process of selecting food for lunch (context). The GenAI 118 may provide coaching feedback to the user 102 indicating that they may make a healthy selection to correct the issue. In another embodiment, sensor data 110 from a forward-facing camera may indicate a restroom sign, and the user agency and capability augmentation system 100 may inform a user 102 with a disability that a restroom is nearby if they have been seated for an extended period of time.

### Additional System Embodiment Details

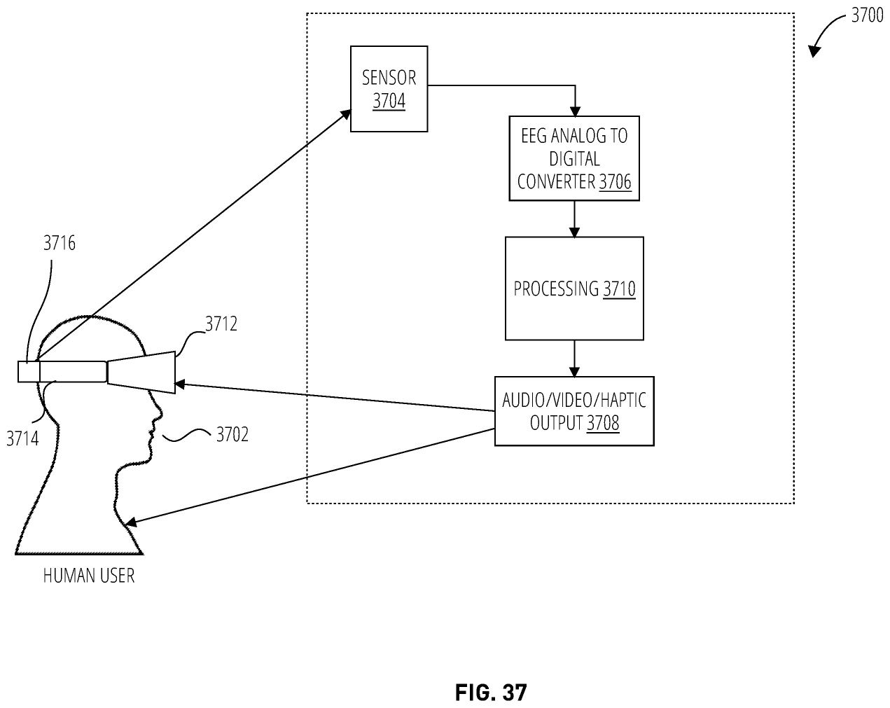

FIG. 37 illustrates an embodiment of a BCI+AR environment 3700. The BCI+AR environment 3700 comprises a sensor 3704, an EEG analog to digital converter 3706, an Audio/Video/Haptic Output 3708, a processing 3710, a strap 3714, an augmented reality glasses 3712, a human user 3702, and a BCI 3716. A human user 3702 is wearing BCI 3716, which is part of a headset. When the human user 3702 interacts with the environment, the sensor 3704, located within the BCI 3716, reads the intentions and triggers the operating system. The EEG analog to digital converter 3706 receives the sensor 3704 output (e.g., intention). EEG analog to digital converter 3706 transforms the sensor output into a digital signal which is sent to processing 3710. The signal is then processed, analyzed and mapped to an Audio/Video/Haptic Output 3708 and displayed on the augmented reality glasses 3712.

In an embodiment, strap 3714 is a head strap for securing the AR+BCI to the human head. In some embodiments, such as an implantable BCI, and AR system, the strap may not be used. The strapless system may use smart glasses or contact lenses. There may be multiple sensors, but no less than one sensor, in different embodiments. After seeing the output, the user may have different bio-signals from the brain, and as such this is a closed-loop biofeedback system. As the user focuses more on the SSVEP stimuli, the audio may feedback by frequency, power (volume), and selected cue audio to assist the human in reinforcing their focus on the stimuli. This may also occur with the vibration type and intensity of the haptics, as well as additional peripheral visual cues in the display. This feedback is independent from the audio and haptics that may play back through the AR headset via a smartphone. It is even possible to remotely add to the sensory mix that of olfactory (smell) feedback that actually travels through entirely different parts of the brain that has been shown to be one of the strongest bio-feedback reinforcements in human cognitive training.

As a non-limiting example, when someone uses the BCI for the first time, they are considered a “naïve” user, or one whose brain has never been trained with this kind of user interface. As a user continues to use it, their brain becomes less naïve and more capable and trained. They may become quicker and quicker at doing it. This is reinforcement learning—the BCI enables someone to align their intention and attention to an object and click it.

In an embodiment, to enrich the user interface experience, multiple feedback modalities (auditory, visual, haptic, and olfactory) may be available for choosing the most advantageous feedback modality for the individual or for the type of training. For example, when an appropriate brain wave frequency is generated by the user, real-time feedback about the strength of this signal may be represented by adjusting the intensity and frequency of the audio or haptic feedback. In addition, the possibility of using multimodal feedback means that multiple sensory brain regions are stimulated simultaneously, which enhances the neural signal and representation of feedback, thereby accelerating learning and neural plasticity.

An advantage of using odors as reinforcers may be due to the direct link between the brain areas that sense smell (olfactory cortex) and those that form memories (hippocampus) and produce emotions (amygdala). Odors may strengthen memory encoding, consolidation, and trigger recall.

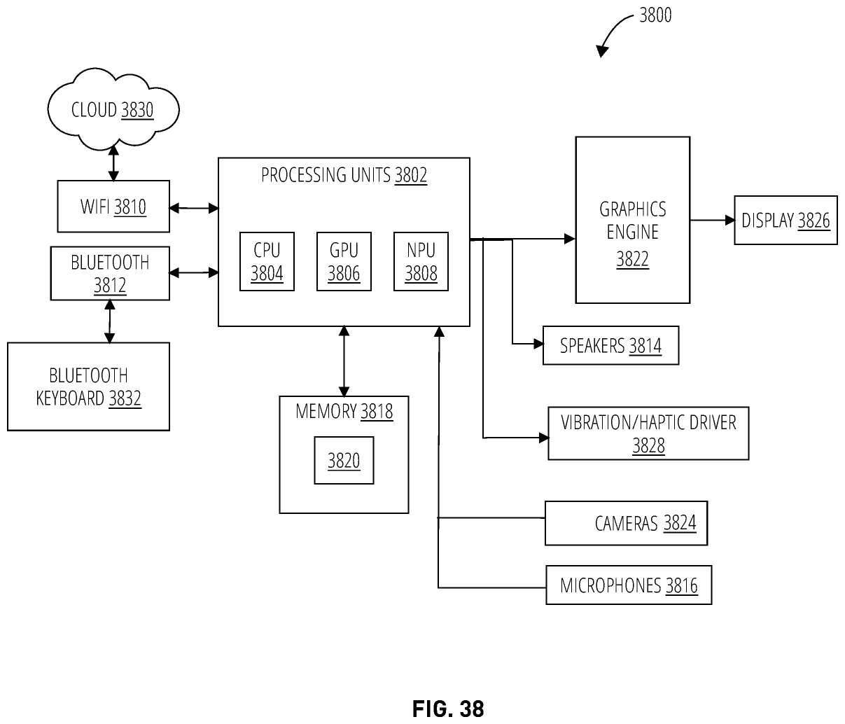

FIG. 38 illustrates components of an exemplary augmented reality device logic 3800. The augmented reality device logic 3800 comprises a graphics engine 3822, a camera 3824, processing units 3802, including one or more central processing units CPU 3804, graphical processing units GPU 3806, and/or neural processing units NPU 3808, a WiFi 3810 wireless interface, a Bluetooth 3812 wireless interface, speakers 3814, microphones 3816, one or more memory 3818, logic 3820, a visual display 3826, and vibration/haptic driver 3828.

The processing units 3802 may in some cases comprise programmable devices such as bespoke processing units optimized for a particular function, such as AR related functions. The augmented reality device logic 3800 may comprise other components that are not shown, such as dedicated depth sensors, additional interfaces, etc.

Some or all of the components in FIG. 38 may be housed in an AR headset. In some embodiments, some of these components may be housed in a separate housing connected or in wireless communication with the components of the AR headset. For example, a separate housing for some components may be designed to be worn or a belt or to fit in the wearer's pocket, or one or more of the components may be housed in a separate computer device (smartphone, tablet, laptop or desktop computer etc.) which communicates wirelessly with the display and camera apparatus in the AR headset, whereby the headset and separate device constitute the full augmented reality device logic 3800. A user may also communicate with the AR headset via a Bluetooth keyboard 3832. Additionally, the AR headset may communicate with the cloud 3830 via WiFi 3810 or cellular connection.

The memory 3818 comprises logic 3820 to be applied to the processing units 3802 to execute. In some cases, different parts of the logic 3820 may be executed by different components of the processing units 3802. The logic 3820 typically comprises code of an operating system, as well as code of one or more applications configured to run on the operating system to carry out aspects of the processes disclosed herein.

FIG. 39 is a block diagram of nonverbal multi-input and feedback device 3900 of a nonverbal multi-input and feedback device such as herein. It may be a block diagram of a portion of the device such as a processing portion of the device. FIG. 39 may be a high-level system architecture block diagram that helps explain the major building blocks. Block diagram of nonverbal multi-input and feedback device 3900 may be applied to the overall system (e.g., multiple devices used as inputs), into a common universal application interface that enables the application 3902 to synchronize data coming from multiple devices and process signals with meta data, plus vocabulary and output logic to a plurality of output methods. FIG. 20 takes this to a finer level of detail.

In the center of block diagram of nonverbal multi-input and feedback device 3900 is the application 3902 or main processing block. To the left is the multimodal input and intent detection 3904 block which receives and processes user inputs from sensors (e.g., based on user input received by the sensors) such as touch 3912; bio-signals 3914; keyboard 3916; facial tracking 3918; eye and pupil tracking 3920; and alternative inputs 3922. This multimodal input and intent detection 3904 block feeds the processing from these inputs to the application 3902.

Above is a context awareness 3906 block which receives and processes metadata inputs from sensors such as biometrics 3924; environment 3926; object recognition 3928; facial recognition 3930; voice recognition 3932; date and time 3934; history 3936; location 3938; proximity 3940; and other metadata 3942 inputs. This context awareness 3906 block feeds the processing from these inputs to the application 3902.

To the right is an output and action 3910 block which sends outputs to displays, computing devices, controllers, speakers and network communication devices such as flat screen flat screen display 3944; augmented/virtual reality 3946; virtual AI assistant 3948; synthesized voice 3950; prosthetic device 3952; social media and messaging 3954; media consumption 3956; and other output. The outputs may include control commands and communication sent to other computing devices. they may include text, graphics, emoji, and/or audio.

Below is a GenAI 3908 block that provides a lexicon or vocabulary in the selected language to the application. FIG. 39 may also be applied to a single sensory device unto itself. This may be a “Big Idea” in so far as the architecture may scale from a single closed-loop system as well as combinations of sensory I/O devices. It may be a system of systems that scale up, down and play together.

The system in block diagram of nonverbal multi-input and feedback device 3900 comprises one (or more) sensory input, one intent detection application programming interface (API), one application, one (or more) meta data, one (or more) vocabulary, one (or more) output and action method, and one (or more) output/actuation system or device. It may be thought of as a universal “augmented intelligence” engine that takes inputs, enriches them with extra meaning, and directs the output based on instructions for the enriched information.

In a simple embodiment of diagram, a user sees a symbol or button that means “help”. and presses it, and the device says “help”. In a more complicated embodiment of block diagram of nonverbal multi-input and feedback device 3900, a user sees a symbol or button that means “help” and presses it. Here, rather than the device saying “help,” it learns that the user is connected to a caregiver with logic to send urgent matters to that person via text or instant message when away from home. The device may geolocation data that indicates the user is away from home; tag the communication with appended contextual information; and its output and action logic tell the system to send a text message to the caregiver with the user's location in a human-understandable grammatically correct phrase “Help, I'm in Oak Park” including the user's Sender ID/Profile and coordinates pinned on a map.

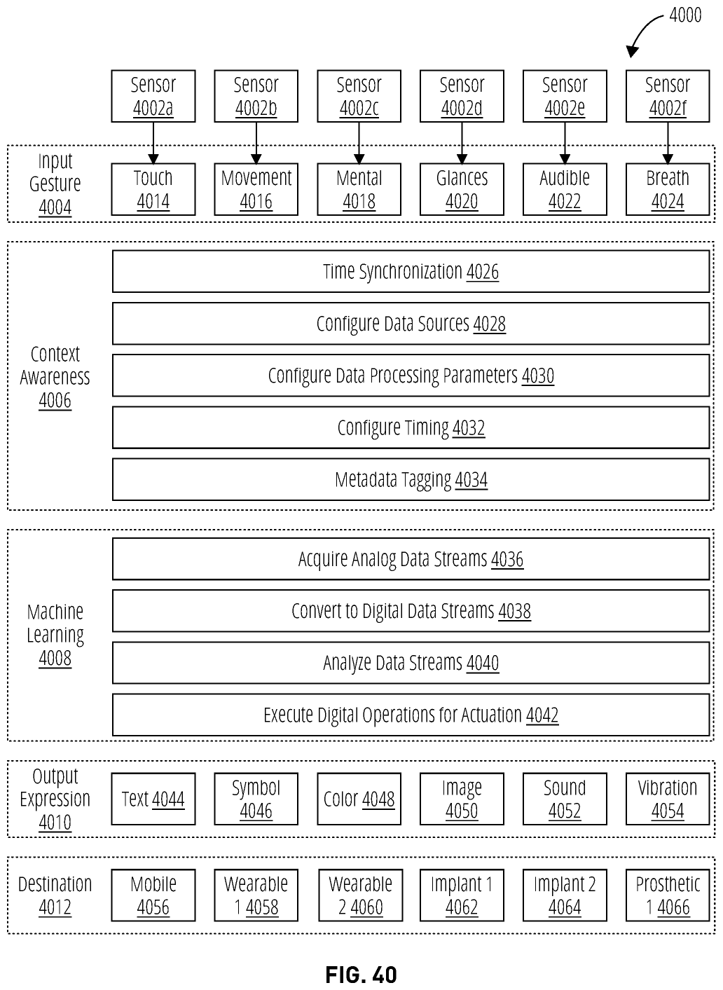

FIG. 40 is a block diagram of a single framework of a nonverbal multi-input and feedback device 4000 such as herein. The block diagram of a single framework of a nonverbal multi-input and feedback device 4000 may be of a single framework for translating diverse sensor inputs into a variety of understandable communication and command outputs for a nonverbal multi-input and feedback device such as herein. The single framework of a nonverbal multi-input and feedback device comprises sensors 4002a-4002f, input gestures 4004, context awareness 4006, machine learning 4008, output expressions 4010, and destinations 4012. Input gestures 4004 may include touch 4014, movement 4016, mental 4018, glances 4020, audible 4022, and breath 4024. Context awareness 4006 may include time synchronization 4026, configure data sources 4028, configure data processing parameters 4030, configure timing 4032, and metadata tagging 4034. Machine learning 4008 may include an acquire analog data streams 4036, convert to digital data streams 4038, analyze data streams 4040, and execute digital operations for actuation 4042. Output expressions 4010 may include text 4044, symbol 4046, color 4048, an image 4050, sound 4052, and vibration 4054. Destinations 4012 may include a mobile 4056, a wearable 1 4058, a wearable 2 4060, an implant 1 4062, an implant 2 4064, and a prosthetic 1 4066.

FIG. 40 may describe in more detail what kind of processing is happening within and across the blocks of FIG. 39. Specifically, the left intention signals being combined with context awareness metadata to enrich the data in order to determine the logic of the output and action. FIG. 40 may include the description of the GenAI 3908 and application 3902 boxes of FIG. 39, though not shown. It may be a block diagram of a portion of the device such as a processing portion of the device. In the framework, input from the sensors 4002a-4002f (e.g., due to input received by the sensors) are received by or as an input gesture 4004. In the framework, context awareness 4006 awareness is used to interpret or determine the user gesture or intent from the inputs received. In the framework machine learning 4008 is used to interpret or determine the user gesture or intent from the inputs received. In the framework, output expression 4010 is used to determine the outputs, such as control commands and communication sent to other computing devices that include text, graphics, emoji, and/or audio. In the framework, destination 4012 is used to determine where the outputs are sent, such as to what other computing devices the command and/or communications are to be sent (such as by the network). The user's Primary and Secondary language preferences are accessed during the processing of intention data which is stored in the GenAI 3908 subsystem such as shown in FIG. 39, and may be accessed in the context awareness 4006, machine learning 4008 and output and action 3910 systems and methods in FIG. 39 and FIG. 40.

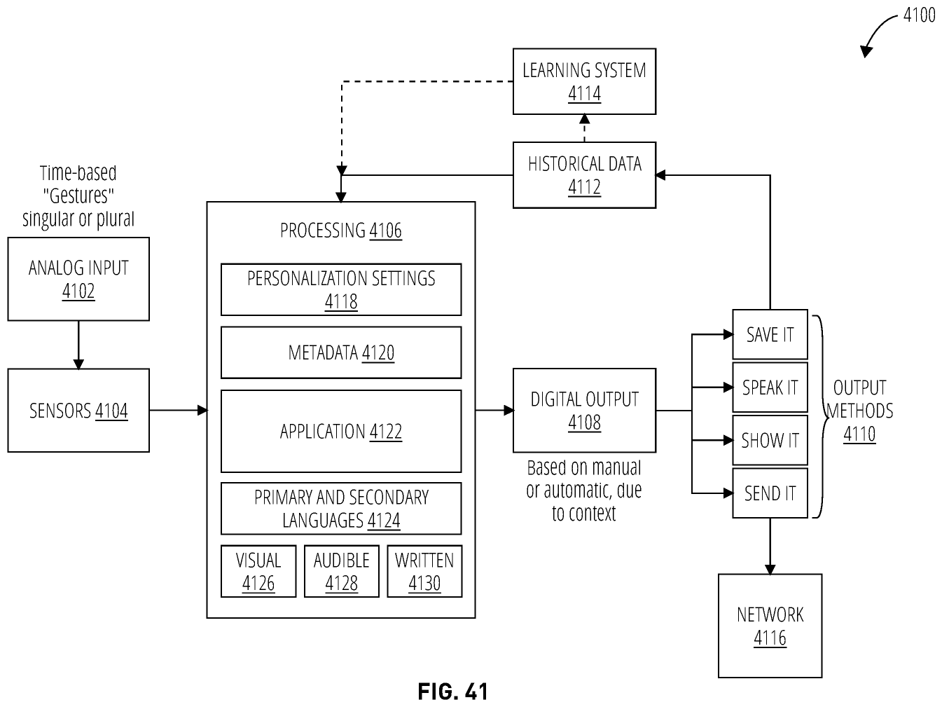

FIG. 41 illustrates a block diagram of nonverbal multi-input and feedback device 4100 in one embodiment. The block diagram of nonverbal multi-input and feedback device 4100 shows a system comprising analog input 4102, sensors 4104, processing 4106, digital output 4108, and output methods 4110 that may be performed with the digital output 4108.

The system illustrated may include an application programming interface (API) that is interoperable with multiple types of analog input 4102 from the sensors 4104. The system illustrated may also comprise a real-time clock for tracking, synchronizing, and metadata 4120 tagging of data streams and analog inputs 4102. The system further comprises a subsystem for data storage and management, for historical data 4112 in some embodiments. The system may comprise a subsystem for personalization settings 4118, as well as a subsystem for sourcing and integrating metadata 4120 into the application 4122 and data stream. The system may further comprise a software application 4122. In some embodiments, the system may include a graphical user interface (GUI) for the software application for the user. In other embodiments, the system may include a GUI for the software application for others who are connected to a system user.

A subsystem of the system may include processing for visual 4126, audible 4128, and written 4130 languages. This language subsystem may differentiate between the user's primary and secondary languages 4124. The language subsystem may set the secondary language manually or automatically. Attributes processed by visual 4126, audible 4128, and written 4130 language subsystems may include but not be limited to color, image, graphics, audible tones, phonemes, dialects, jargon, semantics, tonality, and written characters. In one embodiment, the language subsystems may consist of a suitably trained generative AI model.

The system may include a subsystem of digital outputs 4108 and output methods 4110, that may be configured either manually or automatically. The variety of output methods 4110 may include a network 4116 interface connection. The system may comprise a subsystem for managing data transfer over the network 4116.

The system in some embodiments may comprise a historical data 4112 subsystem for closed-loop machine learning of the system and subsystems and the sensory devices being used with the system. In some embodiments, improved models, algorithms and software may be pushed from the learning system 4114 to update and be used within the system and subsystems and the sensory devices being used with the system.

In one embodiment, the system and subsystems may operate entirely on a sensory device. In one embodiment, the system and subsystems may operate partially on a sensory device and partially distributed to other devices or the cloud. In one embodiment, the system and subsystems may operate entirely distributed on other devices or the cloud.

The system of FIG. 41 may be one embodiment of a fully self-contained brain computer interface in a wireless headset, comprising an augmented reality display as part of the digital output 4108, at least two sensors 4104 for reading a bio-signal from a user as analog input 4102, at least one processing 4106 module for the augmented reality display, at least one biofeedback device that produces at least one of a visual, audible, and tactile effect in communication with the processing module to provide feedback to the user, a wireless network interface that transmits and receives data to and from other devices over the processing 4106, wherein the data is at least one of stored, passed through, and processed on the fully self-contained BCI, as part of the output methods 4110, a battery, wherein the battery provides power to one or more of the augmented reality display, the at least two sensors, the processing module, and the at least one biofeedback device, at least one of onboard storage or remote storage with enough memory to store, process and retrieve the data, and a printed circuit board.

Bio-signals from the user may comprise at least one of EEG, ECG, functional near infrared spectroscopy (fNIRS), Magnetoencephalography (MEG), EMG, EOG, and Time-Domain variants (TD-) of these bio-signal processing methods. Bio-signals may also comprise a visually evoked potential, an audio evoked potential, a haptic evoked potential, and a motion evoked potential, and other bio-signals from multiple sources attached to other body parts other than a user's head.

The at least one processing module for the augmented reality display may include a processor that renders a stimulation effect. This stimulation effect may be at least one of a timed visual stimulation on the augmented reality display, a timed audio stimulation, and a haptic stimulation on the fully self-contained BCI configured to evoke a measurable response in a user's brain. The processing module may include a processor that analyzes and maps the bio-signal into a digital command. This digital command may include at least one of instructions for a visual output configured for displaying on the augmented reality display and instructions for triggering a visual effect. The processing module may be embodied as the processing units 3802 introduced in FIG. 38.

The printed circuit board may include at least one of the at least two sensors, the processing module, the at least one biofeedback device, the battery, and combinations thereof. The printed circuit board may be configured to emulate a Bluetooth keyboard and send output data to at least one of a mobile device, a computer, and the augmented reality display. The output data may include at least one of a letter, a character, a number, and combinations thereof.

Processing performed by the processing module may include the visually evoked potential, the audio evoked potential, and the haptic evoked potential. The bio-signal is processed and analyzed in real-time. The processing module may have different modes, including raw, simmer, and cooked modes, a human interface device-keyboard mode, and combinations thereof. The system may also have a strapless mode, wherein the fully self-contained BCI uses smart glasses or smart contact lenses, an implantable brain computer interface, and an AR system.

The raw mode may stream a full EEG sensor stream of data for further processing locally on device or remotely in a cloud via a mobile or desktop internet connected device that may filter, recognize, or interact with the full EEG sensor stream of data. The cooked mode may comprise a fully processed custom digital command generated by a local recognizer and classifier. This cooked mode data may consist of a sequence of biosignals tokens, as provided by the EEG tokenizer 206, the kinematic tokenizer 208, or the additional tokenizers 210 introduced with respect to FIG. 2. The fully processed custom digital command may be sent to a destination system over the network 4116, per the “send it” output method 4110, and executed on the destination system, with no raw data passed to the user. The recognizer and classifier may be embodied as the recognizer 4624 and classifier 4626 introduced in FIG. 46. The simmer mode may be a hybrid combination between the raw mode and the cooked mode, and the at least one processing module may intersperse a raw data stream with cooked metadata 4120 appended to bio-signal data.

Time domain data may be appended to raw data, cooked data, and simmer data in order for the system to process bio-signal data streams from multiple bio-signal data sources and ensure all bio-signal data streams are synchronized. Metadata from other sensors and data sources may be appended to the raw data, the cooked data, and the simmer data in order for a classifier to alter the command that is sent to execute on a destination system. This classifier may be embodied as the classifier 4626 introduced in FIG. 46. Visual, audible, and tactile sensory frequency stimulators may be appended with metadata from other sensors 4104 and data sources wherein the visual, audible, and tactile sensory frequency stimulators are altered to produce a unique pattern which includes metadata that is decodable by the recognizer and classifier.

The fully self-contained BCI may be electrically detached from the augmented reality display and may be configured to transfer data wirelessly or via a wired connection to an external augmented reality display. The fully self-contained BCI in the wireless headset may be an accessory apparatus that is configured to be temporarily mechanically integrated with another wearable device and configured to transfer data wirelessly or via a wired connection to the other wearable device. The fully self-contained BCI may in another embodiment be permanently mechanically integrated with another wearable device and may transfer data wirelessly or via a wired connection to the other wearable device.

A charging port may be connected to a charging bridge, wherein the charging bridge includes internal circuitry and data management connected to the fully self-contained BCI and the augmented reality display. The internal circuitry may include charging circuitry, thereby allowing charging of both the fully self-contained BCI and the augmented reality display with the charging circuitry.

The fully self-contained BCI may be configured to generate visual, auditory, or haptic stimulations to a user's visual cortex, a user's auditory cortex, and a user's somatosensory cortex, thereby resulting in detectable brain wave frequency potentials that are at least one of stimulated, event-related, and volitionally evoked. The BCI may process the detectable brain wave frequencies, thereby facilitating mapping of bio-signals to digital commands. Stimulation effects and digital commands may be altered with metadata from other sensors or data sources.

The BCI may synchronize bio-signal processing from multiple sensors with a real-time clock such as the real-time clock 4622 introduced in FIG. 46. Digital commands may be associated with a device. The device may be operated according to the digital commands. The BCI may stimulate the user's visual cortex, wherein stimulating includes biofeedback to the user's visual cortex and biofeedback confirmation of the operating of the device. The BCI may stimulate the user's somatosensory cortex, wherein stimulating includes the biofeedback confirmation of the operating of the device. The BCI may stimulate the user's auditory cortex, wherein the stimulating includes biofeedback confirmation of the operating of the device.

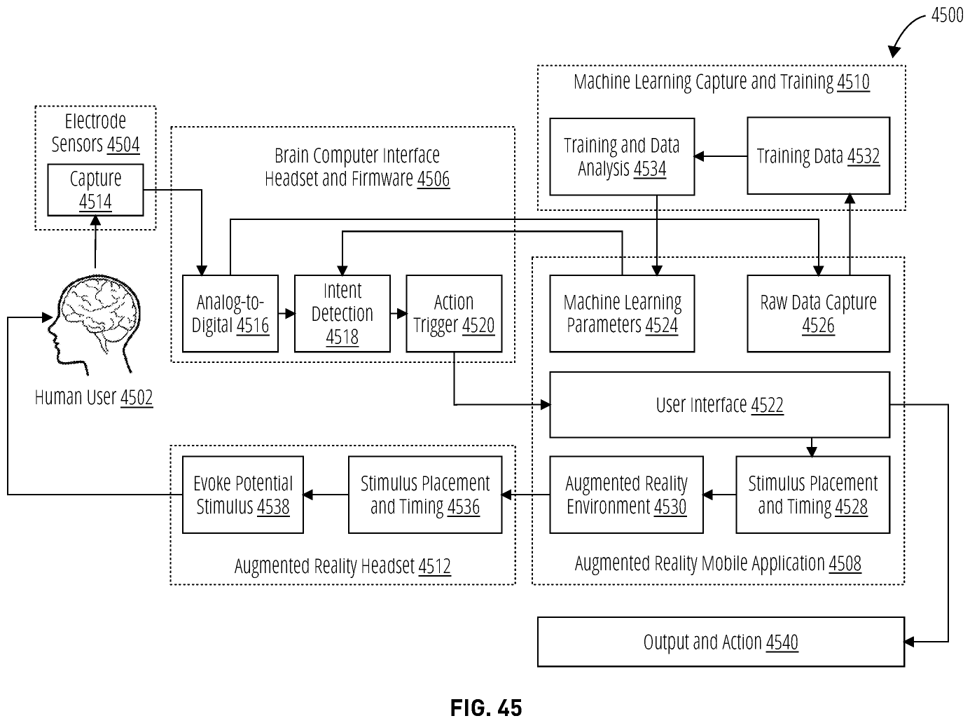

The fully self-contained BCI may be configured to utilize AI machine learning for pattern recognition, classification, and personalization that operates while the fully self-contained BCI is not connected to a network 4116. The AI machine learning may be embodied as the machine learning 4008 introduced in FIG. 40. It may be included in the learning system 4114 of this figure. It may also be supported by the machine learning capture and training 4510 and machine learning parameters 4524 introduced in FIG. 45. The AI machine learning may act as one or more of an auto-tuning dynamic noise reducer, a feature extractor, and a recognizer-categorizer-classifier. AI machine learning training may be applied when the fully self-contained BCI is connected to the network 4116 to create an individualized recognizer-categorizer-classifier. Derived outputs of the AI machine learning training may be stored in a GenAI model in cloud storage or on a mobile computing device having at least one of a wireless connection and a wired connection to the wireless headset and being at least one of mounted on the wireless headset and within wireless network range of the wireless headset. Synthesized insights derived from the AI machine learning and the GenAI may be stored in cloud storage or on the mobile computing device and may be used to generate an individualized executable recognizer-categorizer-classifier downloadable onto the at least one processing 4106 module of the fully self-contained BCI or the mobile computing device via at least one of a wireless connection and a wired connection between the network and a BCI storage device for offline usage without network dependencies. The system may be configured to interface with resource constrained devices including wearable devices, implantable devices, and internet of things (IoT) devices. At least one biofeedback device may be configured to stimulate at least one of a user's central nervous system and peripheral nervous system.

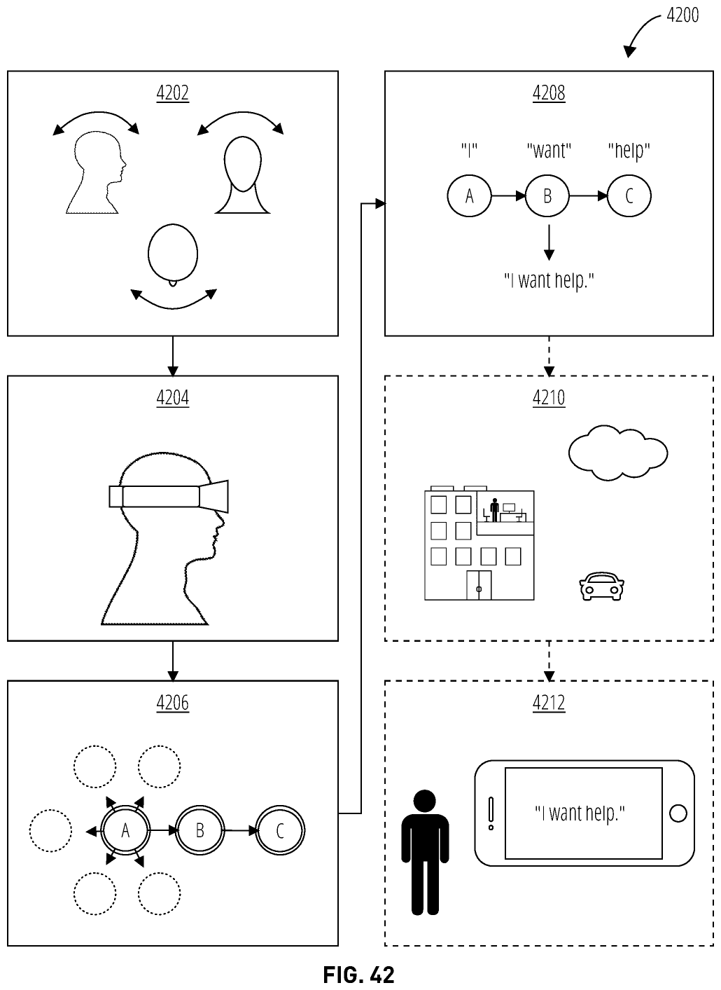

FIG. 42 illustrates a logical diagram of a user wearing an augmented reality headset 4200 that includes a display, speakers and vibration haptic motors and an accelerometer/gyroscope and magnetometer. FIG. 42 shows the flow of activity from head motion analog input 4202 as captured by a headset with head motion detection sensors 4204, through how a user selects options through head motion 4206 and the application creates output based on the user's selected options 4208. On the condition that system detects the user is away from home 4210, FIG. 42 shows that the system may send output to a caregiver via text message 4212.

The user may calibrate the headset based on the most comfortable and stable neck and head position which establishes the X/Y/Z position of 0/0/0. Based on this central ideal position, the user interface is adjusted to conform to the user's individual range of motion, with an emphasis of reducing the amount of effort and distance needed to move a virtual pointer in augmented reality from the 0/0/0 position to outer limits of their field of view and range of motion. The system may be personalized with various ergonomic settings to offset and enhance the users case of use and comfort using the system. A head motion analog input 4202 may be processed as analog streaming data and acquired by the headset with head motion detection sensors 4204 in real-time, and digitally processed, either directly on the sensory device or via a remotely connected subsystem. The system may include embedded software on the sensory device that handles the pre-processing of the analog signal. The system may include embedded software that handles the digitization and post-processing of the signals. Post-processing may include but not be limited to various models of compression, feature analysis, classification, metadata tagging, categorization. The system may handle preprocessing, digital conversion, and post-processing using a variety of methods, ranging from statistical to machine learning. As the data is digitally post-processed, system settings and metadata may be referred to determine how certain logic rules in the application are to operate, which may include mapping certain signal features to certain actions. Based on these mappings, the system operates by sending these post-processed data streams as tokens to the GenAI models and may include saving data locally on the sensory device or another storage device, streaming data to other subsystems or networks.

In the case illustrated in FIG. 42, the user is looking at a display that may include characters, symbols, pictures, colors, videos, live camera footage or other visual, oral or interactive content. In this example, the user is looking at a set of “radial menus” or collection of boxes or circles with data in each one that may be a symbol, character, letter, word or entire phrase. The user has been presented a set of words that surround a central phrase starter word in the middle like a hub and spoke to choose from based on typical functional communication with suggested fringe words and access to predictive keyboard, structured and unstructured language. The user selects options through head motion 4206 and may rapidly compose a phrase by selecting the next desired word presented in the radial menus or adding a new word manually via another input method. The user traverses the interface using head movement gestures, similar to 3-dimensional swipe movements, to compose communication. The user progressively chooses the next word until they're satisfied with the phrase they've composed and may determine how to actuate the phrase. Algorithms may be used to predict the next character, word, or phrase, and may rearrange or alter the expression depending on its intended output including but not limited to appending emoji, symbols, colors, sounds or rearranging to correct for spelling or grammar errors. The user may desire for the phrase to be spoken aloud to a person nearby, thus selecting a “play button” or simply allowing the sentence to time out to be executed automatically. The application creates output based on the user's selected options 4208. If they compose a phrase that is a control command like “turn off the lights”, they may select a “send button” or may, based on semantic natural language processing and understanding, automatically send the phrase to a third party virtual assistant system to execute the command, and turn off the lights. The potential use of metadata, in this example, could simply be geolocation data sourced from other systems such as a geographic information system (GIS) or a global positioning system (GPS) data or WiFi data, or manually personalized geofencing in the application personalization settings, where the system would know if the user is “at home” or “away from home”. On condition that system detects the user is away from home 4210, for example, the metadata may play a role in adapting the language being output to reflect the context of the user. For instance, the system could be configured to speak aloud when at home but send output to a caregiver via text message 4212 and append GPS coordinates when away from home. The system may support collecting and processing historical data from the sensory device, system, subsystems, and output actions to improve the performance and personalization of the system, subsystems, and sensory devices.

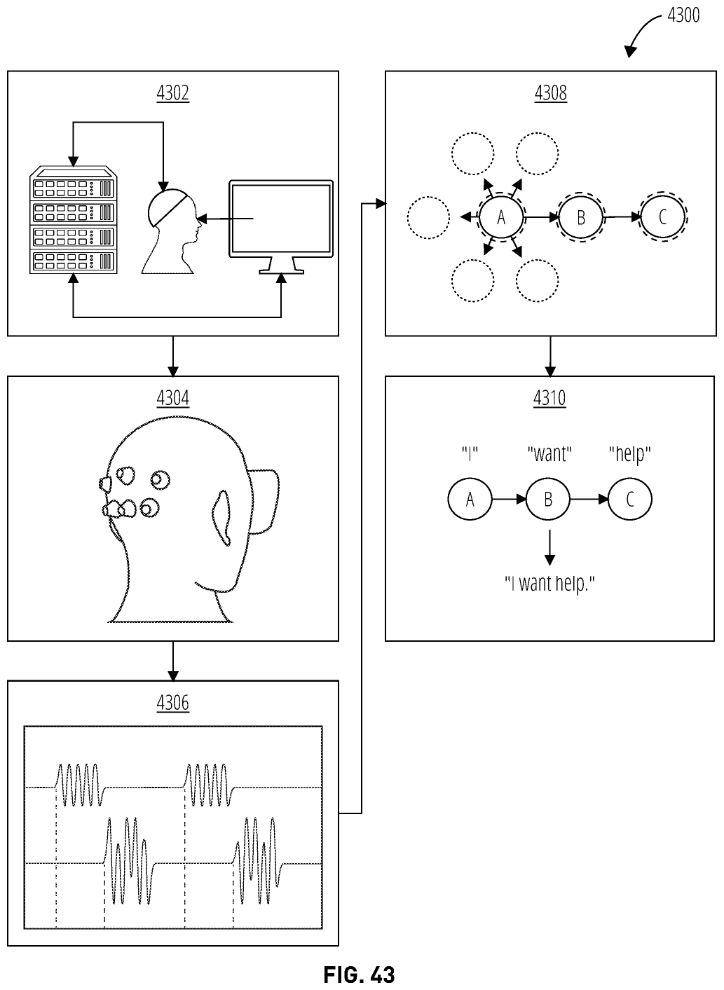

FIG. 43 illustrates a logical diagram of a user wearing an augmented reality headset 4300, in which user wears an EEG-based brain-computer interface headset 4302 containing electrodes that are contacting the scalp 4304. FIG. 43 shows that streaming analog data may be acquired from the brainwave activity 4306. In this manner, the user may be presented a set of words to choose from 4308, compose a phrase, and select what action the system takes using the phrase they've composed 4310.

A user wears an EEG-based brain-computer interface headset 4302 containing electrodes that are contacting the scalp 4304. The electrodes are connected to an amplifier and analog-to-digital processing pipeline. The sensory device (BCI) acquires streaming electrical current data measured in microvolts (uV). The more electrodes connected to the scalp and to the BCI, the more streaming analog data may be acquired from the brainwave activity 4306. The analog streaming data is acquired by the electrodes, pre-processed through amplification, and digitally processed, either directly on the sensory device or via a remotely connected subsystem. The system may include embedded software on the sensory device that handles the pre-processing of the analog signal. The system may include embedded software that handles the digitization and post-processing of the signals. Post-processing may include but not be limited to various models of compression, feature analysis, classification, metadata tagging, categorization. The system may handle preprocessing, digital conversion, and post-processing using a variety of methods, ranging from statistical to machine learning. As the data is digitally post-processed, system settings and metadata may be referred to determine how certain logic rules in the application are to operate, which may include mapping certain signal features to certain actions. Based on these mappings, the system operates by executing commands and may include saving data locally on the sensory device or another storage device, streaming data to other subsystems or networks.

In the case illustrated in FIG. 43, the user is looking at a display that may include characters, symbols, pictures, colors, videos, live camera footage or other visual, oral or interactive content. In this example, the user is looking at a group of concentric circles, arranged in a radial layout, with characters on each circle. The user has been presented a set of words to choose from 4308 based on typical functional communication with suggested fringe words and access to predictive keyboard and may rapidly compose a phrase by selecting the next desired word presented in the outer ring of circles or adding a new word manually. The user progressively chooses the next word until they're satisfied with the phrase they've composed 4310 and may determine how to actuate the phrase. GenAI may be used to predict the next character, word, or phrase, and may rearrange or alter the expression depending on its intended output including but not limited to appending emoji, symbols, colors, sounds or rearranging to correct for spelling or grammar errors. The user may desire for the phrase to be spoken aloud to a person nearby, thus selecting a “play button” or simply allowing the sentence to time out to be executed automatically. If they compose a phrase that is a control command like “turn off the lights”, they may select a “send button” or may, based on semantic natural language processing and understanding, automatically send the phrase to a third party virtual assistant system to execute the command, and turn off the lights. The potential use of metadata, in this example, could simply be geolocation data sourced from other systems such as GIS or GPS data or WiFi data, or manually personalized geofencing in the application personalization settings, where the system may know if the user is “at home” or “away from home”. In this case, the metadata may play a role in adapting the language being output to reflect the context of the user. For instance, the system could be configured to speak aloud when at home but send to a caregiver via text message and append GPS coordinates when away from home. The system may support collecting and processing historical data from the sensory device, system, subsystems, and output actions to improve the performance and personalization of the system, subsystems, and sensory devices.

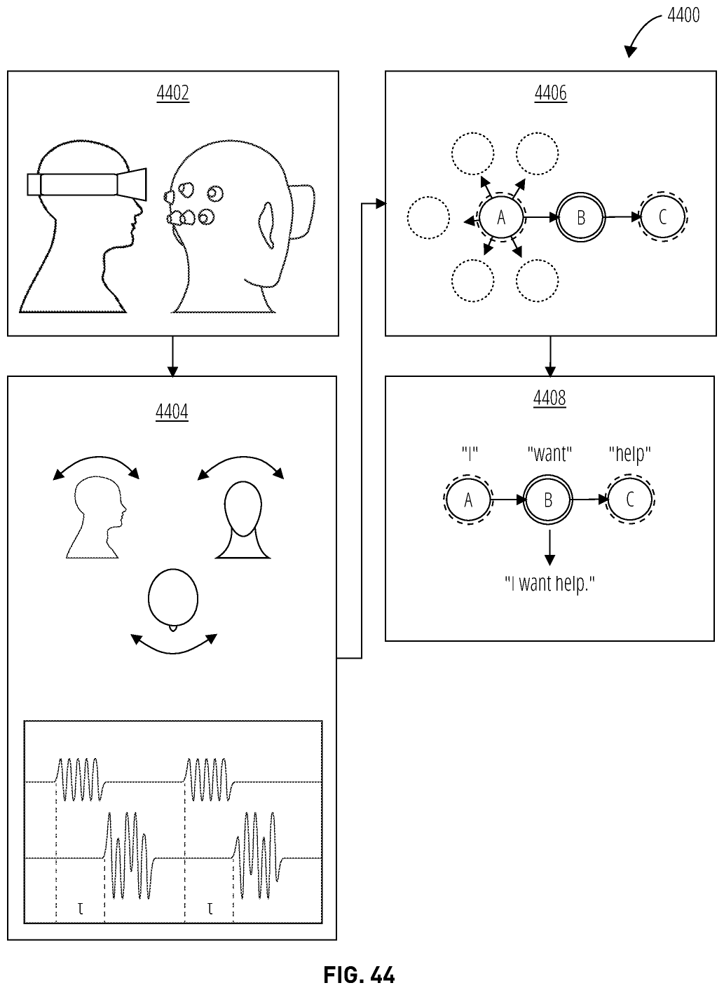

FIG. 44 illustrates a diagram of a use case including a user wearing an augmented reality headset 4400, in which a user wears an augmented reality headset combined with a brain computer interface 4402, having the capabilities described with respect to FIG. 42 and FIG. 43. Both head motion analog input and brainwave activity 4404 may be detected and may allow a user to select from a set of words to choose from 4406, as well as what to do with the phrase they've composed 4408 by selecting those words.

A user is wearing an augmented reality headset combined with a brain computer interface on their head. The headset contains numerous sensors as a combined sensory device including motion and orientation sensors and temporal bioelectric data generated from the brain detected via EEG electrodes contacting the scalp of the user, specifically in the regions where visual, auditory and sensory/touch is processed in the brain. The AR headset may produce visual, auditory or haptic stimulation that is detectible via the brain computer interface, and by processing brainwave data with motion data, the system may provide new kinds of multi-modal capabilities for a user to control the system. The analog streaming data is acquired by the Accelerometer, Gyroscope, Magnetometer and EEG analog-to-digital processor, and digitally processed, either directly on the sensory device or via a remotely connected subsystem. The system may include embedded software on the sensory device that handles the pre-processing of the analog signal. The system may include embedded software that handles the digitization and post-processing of the signals. Post-processing may include but not be limited to various models of compression, feature analysis, classification, metadata tagging, categorization. The system may handle preprocessing, digital conversion, and post-processing using a variety of methods, ranging from statistical to machine learning. As the data is digitally post-processed, system settings and metadata may be referred to determine how certain logic rules in the application are to operate, which may include mapping certain signal features to certain actions. Based on these mappings, the system operates by executing commands and may include saving data locally on the sensory device or another storage device, streaming data to other subsystems or networks.

In the case illustrated in FIG. 44, the user is looking at a display that may include characters, symbols, pictures, colors, videos, live camera footage or other visual, oral or interactive content. In this example, the user is looking at a visual menu system in AR with certain hard to reach elements flickering at different frequencies. The user has been presented a set of items to choose from based on typical functional communication with suggested fringe words and access to predictive keyboard and may rapidly compose a phrase by selecting the next desired word presented in the AR head mounted display or adding a new word manually. Enabling the user affordances of extra-sensory reach of visible objects out of reach within the comfortable range of motion of neck movement. The user progressively chooses the next word until they're satisfied with the phrase they've composed and may determine how to actuate the phrase. Algorithms may be used to predict the next character, word, or phrase, and may rearrange or alter the expression depending on its intended output including but not limited to appending emoji, symbols, colors, sounds or rearranging to correct for spelling or grammar errors. The user may desire for the phrase to be spoken aloud to a person nearby, thus selecting a “play button” or simply allowing the sentence to time out to be executed automatically. If they compose a phrase that is a control command like “turn off the lights”, they may select a “send button” or may, based on semantic natural language processing and understanding. automatically send the phrase to a third party virtual assistant system to execute the command, and turn off the lights. The potential use of metadata, in this example, could simply be geolocation data sourced from other systems such as GIS or GPS data or WIFI data, or manually personalized geofencing in the application personalization settings, where the system may know if the user is “at home” or “away from home”. In this case, the metadata may play a role in adapting the language being output to reflect the context of the user. For instance, the system could be configured to speak aloud when at home but send to a caregiver via text message and append GPS coordinates when away from home. The system may support collecting and processing historical data from the sensory device, system, subsystems, and output actions to improve the performance and personalization of the system, subsystems, and sensory devices.

FIG. 45 is a flow diagram 4500 showing a closed loop bio-signal data flow for a nonverbal multi-input and feedback device such as herein. It may be performed by inputs or a computer of the device. The flow diagram 4500 comprises a human user 4502, electrode sensors 4504, a brain computer interface headset and firmware 4506, an augmented reality mobile application 4508, machine learning capture and training 4510 that may be performed in an edge, peer, or cloud device, and an augmented reality headset 4512. The electrode sensors 4504 may capture 4514 data that is sent for analog-to-digital 4516 conversion. The digital signal may be used for intent detection 4518 resulting in an action trigger 4520 to a user interface 4522. The digital data may further be sent to raw data capture 4526 and may be used as training data 4532 for training and data analysis 4534. Training and data analysis 4534 may yield machine learning parameters 4524 which may be fed back for use in intent detection 4518. The user interface 4522 may determine stimulus placement and timing 4528, which may be used in the augmented reality environment 4530 created by the augmented reality mobile application 4508. The stimulus placement and timing 4536 resulting in the augmented reality headset 4512 and may evoke potential stimulus 4538 in the human user 4502. The user interface 4522 may also generate an output and action 4540.

The flow diagram 4500 includes computer stimulates visual, auditory and somatosensory cortex with evoked potentials; signal processing of real time streaming brain response; human controls computer based on mental fixation of stimulation frequencies; and system may determine different output or actions on behalf of the user for input data received via one or more sensors of the device. Flow diagram 4500 may apply to a user wearing any of the nonverbal multi-input and feedback devices and/or sensors herein. As a result of this being closed-loop biofeedback and sensory communication and control system that stimulates the brains senses of sight, sound, and touch and reads specific stimulation time-based frequencies, and tags them with metadata in real-time as the analog data is digitized, the user may rapidly learn how to navigate and interact with the system using their brain directly. This method of reinforcement learning is known in the rapid development process of the brain's pattern recognition abilities and the creation of neural plasticity to develop new neural connections based on stimulation and entrainment. This further enables the system to become a dynamic neural prosthetic extension of their physical and cognitive abilities. The merging of context awareness metadata, vocabulary, and output and action logic into the central application in addition to a universal interface for signal acquisition and data processing is what makes this system extremely special. Essentially, this system helps reduce the time latency between detecting cognitive intention and achieving the associated desired outcome, whether that be pushing a button, saying a word or controlling robots, prosthetics, smart home devices or other digital systems.

FIG. 46 is a flow diagram 4600 showing multimodal, multi-sensory system for communication and control 4602 for a nonverbal multi-input and feedback device such as herein. It may be performed by inputs or a computer of the device. The flow diagram 4600 comprises multimodal, multi-sensory systems for communication and control 4602 that includes wireless neck and head tracking 4604 and wireless brain tracking 4606. The multimodal, multi-sensory system for communication and control 4602 may further comprise central sensors 4608 for EEG, peripheral sensors 4610 such as EMG, EOG, ECG, and others, an analog to digital signal processor 4612 processing data from the central sensors 4608, and an analog to digital signal processor 4614 processing data from the peripheral sensors 4610. The analog to digital subsystem 4616 and sensor service subsystem 4618 manage output from the analog to digital signal processor 4612 and the analog to digital signal processor 4614, respectively. Output from the analog to digital subsystem 4616 may be sent to a storage subsystem 4660.

Outputs from the analog to digital subsystem 4616 and sensor service subsystem 4618 go to a collector subsystem 4620, which also receives a real-time clock 4622. The collector subsystem 4620 communicates with a recognizer 4624 for EEG data and a classifier 4626 for EMG, EOG, and ECG data, and data from other sensing. The collector subsystem 4620 further communicates to a wireless streamer 4628 and a serial streamer 4630 to interface with a miniaturized mobile computing system 4636 and a traditional workstation 4632, respectively.

The traditional workstation 4632 and miniaturized mobile computing system 4636 may communicate with a cloud 4634 for storage or processing. The miniaturized mobile computing system 4636 may assist in wireless muscle tracking 4638 (e.g., EMG data) and wireless eye pupil tracking 4640.

A controller subsystem 4642 accepts input from a command queue 4644 which accepts input from a Bluetooth or BT write callback 4650. The BT write callback 4650 may send commands 4646 to a serial read 4648. The controller subsystem 4642 may send output to the controller subsystem 4642 and a peripherals subsystem 4652. The peripherals subsystem 4652 generates audio feedback 4654, haptic feedback 4656, and organic LED or OLED visual feedback 4658 for the user.

The flow diagram 4600 includes synchronizing signals from multiple biosensors including brain, body (see skin colored arm), eye and movement; processing multiple models concurrently for multi-sensory input; and directing and processing biofeedback through peripheral subsystems. Flow diagram 4600 may apply to a user wearing any of the nonverbal multi-input and feedback devices and/or sensors herein.

FIG. 47 is a block diagram 4700 showing an example of cloud processing for a nonverbal multi-input and feedback device such as herein. The block diagram 4700 comprises data authentication 4702, a sensory device and mobile system 4704, a cloud system 4706, and a database 4722. The data authentication 4702 module may be configured to authenticate data and communicate with the sensory device and mobile system 4704 and cloud system 4706. The sensory device and mobile system 4704 may include companion application 4708 and data collection, firmware 4710 and data collection, and data analysis 4712 or raw and processed data. The cloud system 4706 may comprise simple queue service or SQS message queuing 4714, server computing 4716 to analyze raw and process data, clastic computing 4718 to build, train, and test machine learning models, and object storage 4720 for persistent storage of biodata, machine learning, and metadata. The database 4722 stores associations and metadata and is in communication with the cloud system 4706.

Block diagram 4700 has the cloud system, the nonverbal multi-input device and an authorization system. Block diagram 4700 includes: machine learning processing signal data on device; metadata enrichment; push raw and processed data to cloud; cloud application building new models for devices; system updates devices remotely and wirelessly; secure and privacy compliant. This configuration is quite powerful but unassumingly simple in this block diagram.

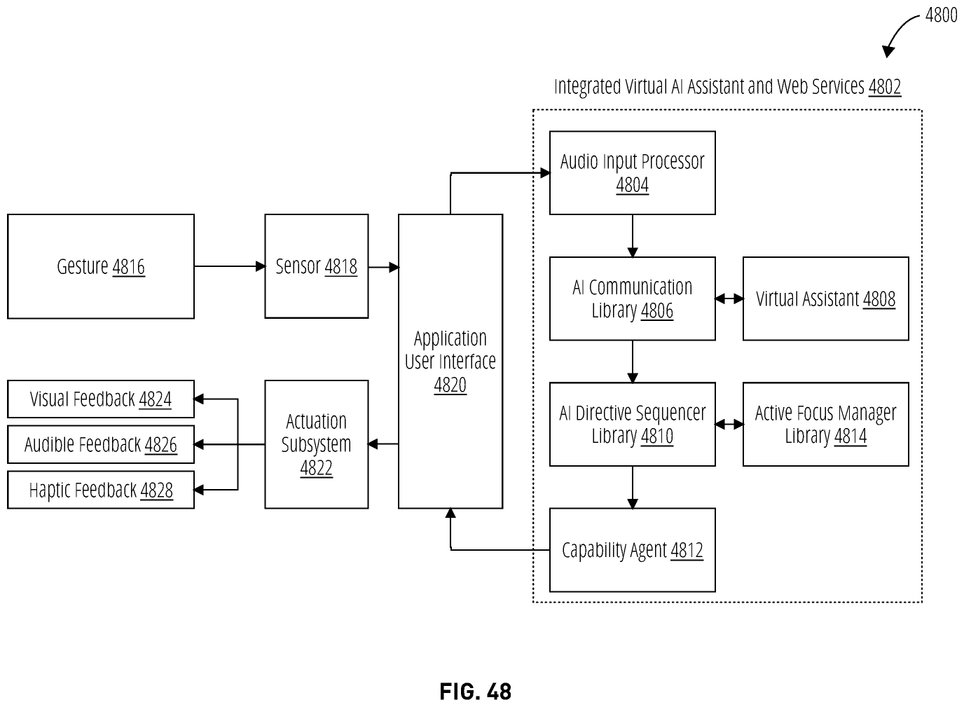

FIG. 48 is a block diagram 4800 showing an example of a system architecture for integrated virtual AI assistant and web services 4802 for a nonverbal multi-input and feedback device such as herein. The block diagram 4800 comprises integrated virtual AI assistant and web services 4802 which may include an audio input processor 4804, an AI communication library 4806, a virtual assistant 4808 such as Alexa, an AI directive sequencer library 4810, a capability agent 4812, and an active focus manager library 4814. A gesture 4816 from a user may be detected by a sensor 4818. An application user interface 4820 may process sensor data and may send data to the audio input processor 4804. The capability agent 4812 may send data back to the application user interface 4820. The application user interface 4820 may signal an actuation subsystem 4822 to provide visual feedback 4824, audible feedback 4826, and haptic feedback 4828.

The block diagram 4800 includes: system manages intention signal acquisition, processing, language composition, and output; in the event where a user wants to send their intention to a virtual assistant (like Alexa, Siri). The blocks outside of the dashed border run on the sensory device, and currently, the blocks inside the dashed line are running in the cloud (e.g., represent a custom configuration for how to use the Alexa service in a cloud architecture.) It may also be possible that all of what is described here as in the cloud may run locally in the sensory device.

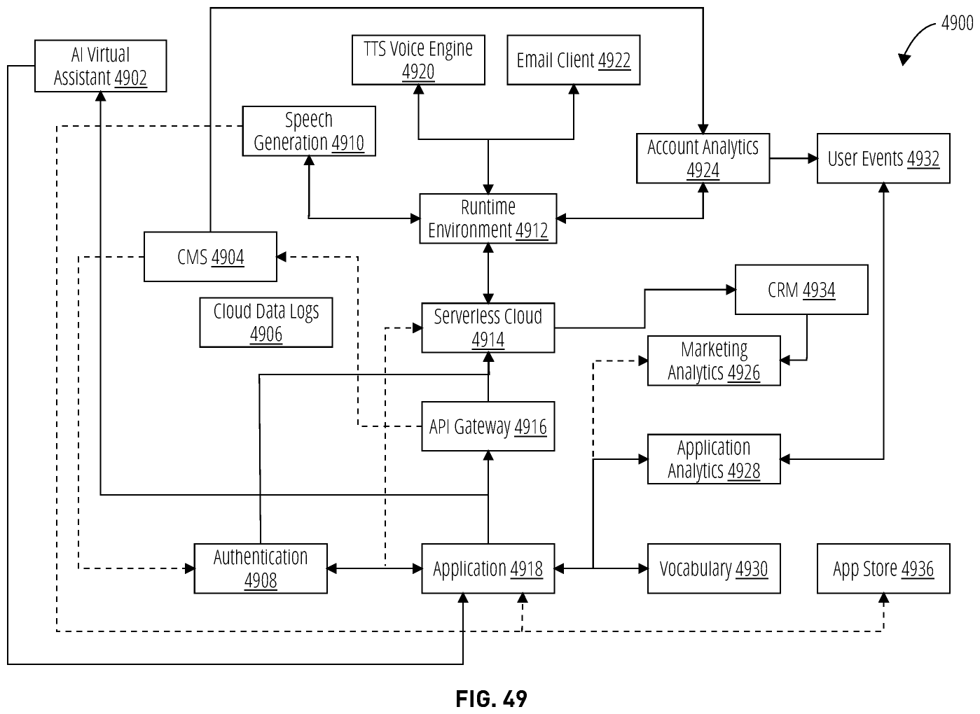

FIG. 49 is a block diagram 4900 showing an example of system operations for a nonverbal multi-input and feedback device such as herein. The block diagram 4900 comprises an AI virtual assistant 4902, such as Alexa, a content management system 4904, cloud data logs 4906, authentication 4908, speech generation 4910, a runtime environment 4912, a serverless cloud 4914, an API gateway 4916, an application 4918, a text-to-speech or TTS voice engine 4920, an email client 4922, account analytics 4924, marketing analytics 4926, application analytics 4928, a vocabulary 4930, user events 4932, a customer relations management 4934, and an app store 4936.

Block diagram 4900 includes: system operation blocks including authentication. This is an example of the complexity of a system operating in the cloud. Everything in this figure is in the cloud, except for the application that is running on the sensory device. The augment/virtual reality application 4918 for the nonverbal multi-input and feedback device may interface with an authentication 4908 module, an API gateway 4916, a vocabulary 4930, application analytics 4928, AI virtual assistant 4902, and marketing analytics 4926. The AI virtual assistant 4902 may communicate back to the application 4918. The application 4918 may also be in direct communication with a serverless cloud 4914 or may communicate with the serverless cloud 4914 through the API gateway 4916. Authentication 4908 may also be in communication with the serverless cloud 4914. The API gateway 4916 further allows the application 4918 to communicate with the content management system 4904, which may be used to store cloud data logs 4906. The content management system 4904 may send data back to the application 4918 through the authentication 4908 module, which may act as a gateway to ensure security and content authorization. Finally, the content management system 4904 may provide data to an account analytics 4924 module. Account analytics 4924 may provide data to a user events 4932 module, which may in turn feed data to application analytics 4928.

The serverless cloud 4914 may allow communication with the runtime environment 4912 and the customer relations management 4934 module. The customer relations management 4934 may provide data for marketing analytics 4926. The runtime environment 4912 may interface with speech generation 4910, a TTS voice engine 4920, an email client 4922, and account analytics 4924. Speech generation 4910 may allow a user to access an app store 4936.

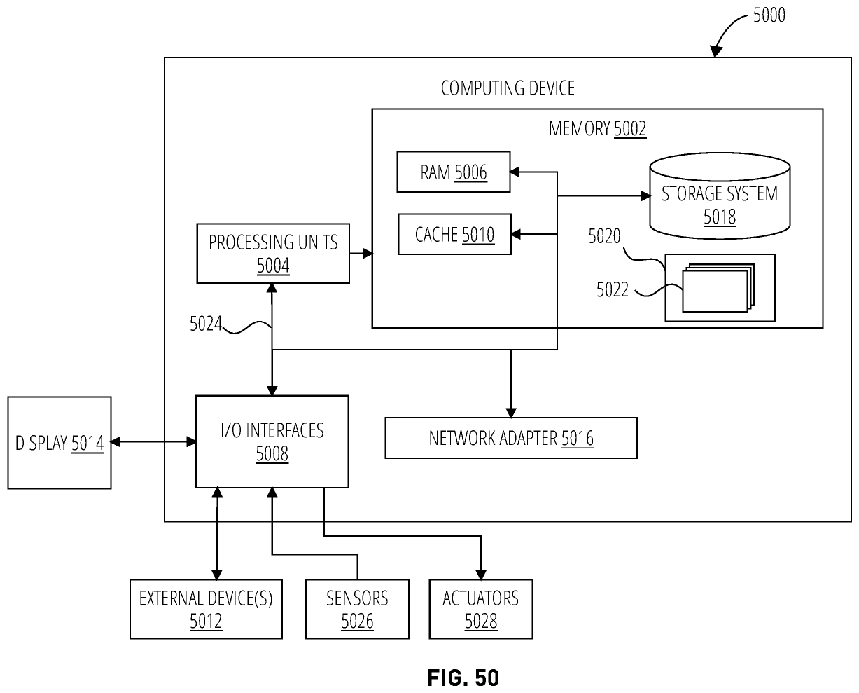

As shown in FIG. 50, a computing device 5000, such as a smart phone or other smart device, is shown in the form of a general-purpose computing device. The components of computing device 5000 may include, but are not limited to, one or more processors or processing units 5004, a system memory 5002, and a bus 5024 that couples various system components including system memory 5002 to processor processing units 5004. Computing device 5000 may include sensors 5026 such as cameras, accelerometers, microphones, etc., and actuators 5028, such as speakers, vibrating or haptic actuators, etc. Computing device 5000 may be a smartphone, a tablet, laptop, desktop computer, or other computing device suitable for implementing the disclosed solution as described herein.

Bus 5024 represents one or more of any of several types of bus structures, including a memory bus or memory controller, a peripheral bus, an accelerated graphics port, and a processor or local bus using any of a variety of bus architectures. By way of example, and not limitation, such architectures include Inter-Integrated Circuit (I2C), Serial Peripheral Interface (SPI), Controller Area Network (CAN), Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

Computing device 5000 typically includes a variety of computer system readable media. Such media may be any available media that is accessible by computing device 5000, and it includes both volatile and non-volatile media, removable and non-removable media.

System memory 5002 may include computer system readable media in the form of volatile memory, such as Random access memory (RAM) 5006 and/or cache memory 5010. Computing device 5000 may further include other removable/non-removable, volatile/non-volatile computer system storage media. By way of example, a storage system 5018 may be provided for reading from and writing to a non-removable, non-volatile magnetic media (not shown and typically called a “hard drive”). Although not shown, a flash drive, a magnetic disk drive for reading from and writing to a removable, non-volatile magnetic disk (e.g., a “floppy disk”), and an optical disk drive for reading from or writing to a removable, non-volatile optical disk such as a compact disc read only memory (CD-ROM), digital versatile disc read only memory (DVD-ROM), or other optical media, may be provided. In such instances, each may be connected to bus 5024 by one or more data media interfaces. As will be further depicted and described below, system memory 5002 may include at least one program product having a set (e.g., at least one) of program modules that are configured to carry out the functions of the disclosure.

Program/utility 5020 having a set (at least one) of program modules 5022 may be stored in system memory 5002 by way of example, and not limitation, as well as an operating system, one or more application programs, other program modules, and program data. Each of the operating system, one or more application programs, other program modules, and program data or some combination thereof, may include an implementation of a networking environment. Program modules 5022 generally carry out the functions and/or methodologies of the disclosure as described herein.

Computing device 5000 may also communicate with one or more external devices 5012 such as a keyboard, a pointing device, a display 5014, etc.; one or more devices that enable a user to interact with computing device 5000; and/or any devices (e.g., network card, modem, etc.) that enable computing device 5000 to communicate with one or more other computing devices. Such communication may occur via I/O interfaces 5008. I/O interfaces 5008 may also manage input from computing device 5000 sensors 5026, as well as output to actuators 5028. Still yet, computing device 5000 may communicate with one or more networks such as a local area network (LAN), a general wide area network (WAN), and/or a public network (e.g., the Internet) via network adapter 5016. As depicted, network adapter 5016 communicates with the other components of computing device 5000 via bus 5024. It may be understood that although not shown, other hardware and/or software components could be used in conjunction with computing device 5000. Examples include, but are not limited to, microcode, device drivers, redundant processing units, external disk drive arrays, redundant array of independent disks (RAID) systems, tape drives, data archival storage systems, etc.

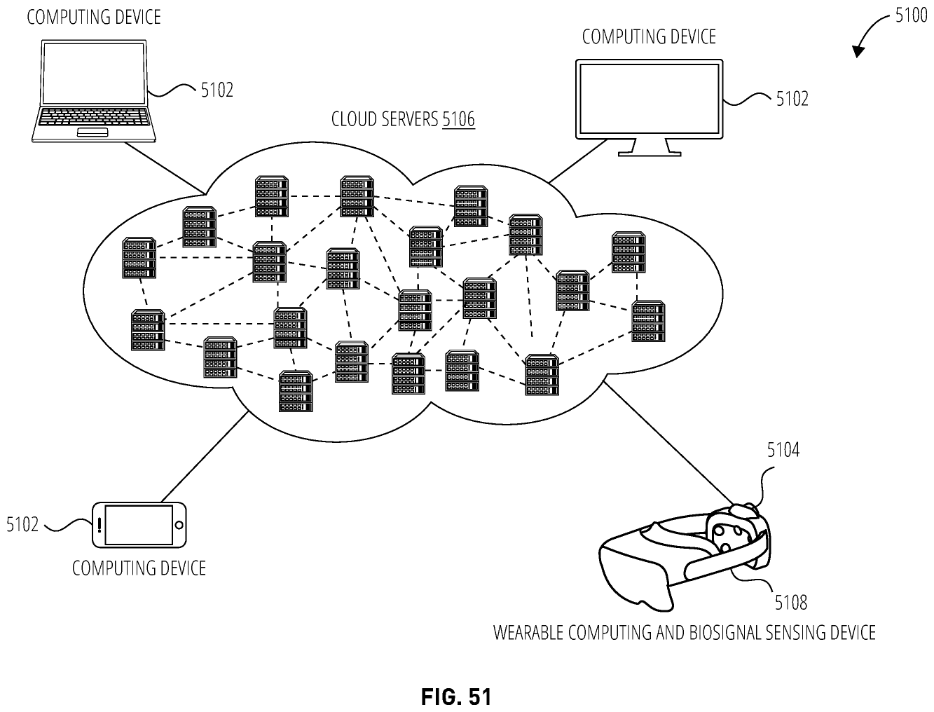

Referring now to FIG. 51, an illustrative cloud computing system 5100 is depicted. “Cloud computing” refers to a model for enabling convenient, on-demand network access to a shared pool of configurable computing resources (e.g., networks, servers, storage, applications, and services) that may be rapidly provisioned and released with minimal management effort or service provider interaction. This cloud model promotes availability and is comprised of at least five characteristics, at least three service models, and at least four deployment models. Examples of commercially hosted cloud computing systems 5100 include Amazon Web Services (AWS), Google Cloud, Microsoft Azure, etc.

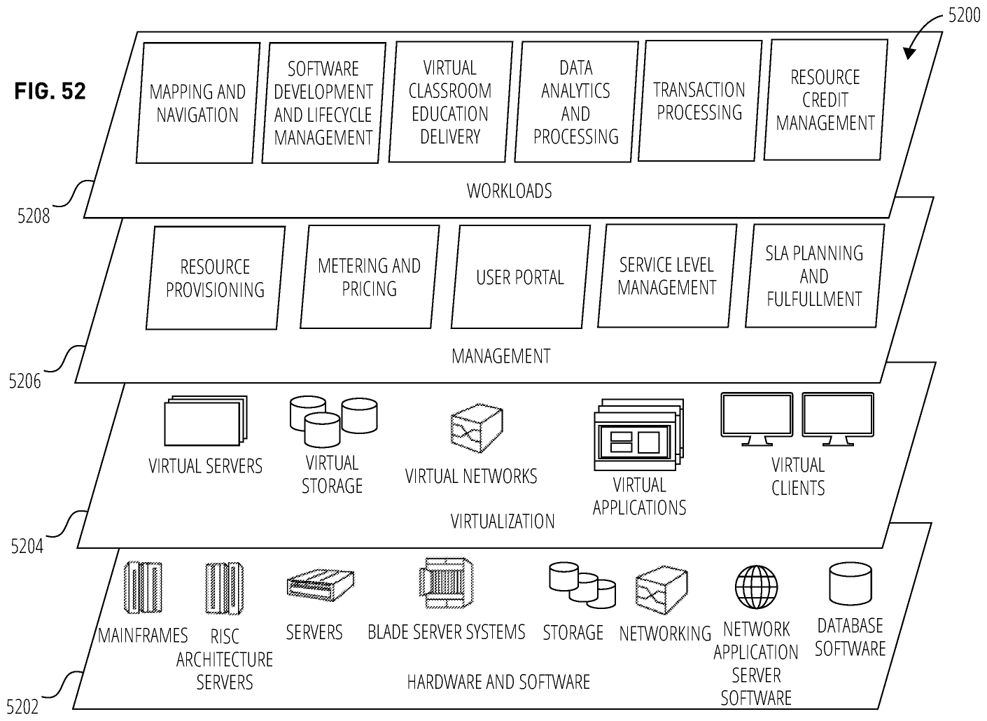

As shown, cloud computing system 5100 may comprise one or more cloud servers 5106, with which computing devices 5102 such as, for example, personal digital assistants (PDAs), smart devices such as smart phones, laptops, desktop computers, and/or wearable computing and biosignal sensing device 5108 brain computer interfaces or BCIs 5104 may communicate. This allows for infrastructure, platforms, and/or software to be offered as services (as described above in FIG. 52) from cloud servers 5106, so as to not require each client to separately maintain such resources. It is understood that the types of computing devices shown in FIG. 51 are intended to be illustrative and not limiting, and that cloud servers 5106 may communicate with any type of computerized device over any type of network and/or network/addressable connection (e.g., using a web browser).

It is to be understood that although this disclosure includes a detailed description on cloud computing, implementation of the teachings recited herein are not limited to a cloud computing environment. Rather, embodiments of the present disclosure are capable of being implemented in conjunction with any other type of computing environment now known or later developed.

It is to be understood that although this disclosure includes a detailed description on cloud computing, implementation of the teachings recited herein are not limited to a cloud computing environment. Rather, embodiments of the present disclosure are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that may be rapidly provisioned and released with minimal management effort or interaction with a provider of the service. This cloud model may include at least five characteristics, at least three service models, and at least four deployment models.

Characteristics are as follows:

On-demand self-service: a cloud consumer may unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities may be rapidly and elastically provisioned, in some cases automatically, to quickly scale out and rapidly released to quickly scale in. To the consumer, the capabilities available for provisioning often appear to be unlimited and may be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage may be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which may include operating systems and applications. The consumer does not manage or control the underlying cloud infrastructure but has control over operating systems, storage, deployed applications, and possibly limited control of select networking components (e.g., host firewalls).

### Deployment Models are as Follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

Referring now to FIG. 52, a set of cloud computing functional abstraction layers 5200 provided by cloud computing systems 5100 such as those illustrated in FIG. 51 is shown. It may be understood in advance that the components, layers, and functions shown in FIG. 52 are intended to be illustrative, and the disclosure is not limited thereto. As depicted, the following layers and corresponding functions are provided:

Hardware and software layer 5202 includes hardware and software components. Examples of hardware components include mainframes, reduced instruction set computer (RISC) architecture based servers, servers, blade servers, storage devices, and networks and networking components. Examples of software components include network application server software and database software.

Virtualization layer 5204 provides an abstraction layer from which the following exemplary virtual entities may be provided: virtual servers; virtual storage; virtual networks, including virtual private networks; virtual applications; and virtual clients.

Management layer 5206 provides the exemplary functions described below. Resource provisioning provides dynamic procurement of computing resources and other resources that are utilized to perform tasks within the Cloud computing environment. Metering and Pricing provide cost tracking as resources are utilized within the cloud computing environment, and billing or invoicing for consumption of these resources. In one example, these resources may comprise application software licenses. Security provides identity verification for users and tasks, as well as protection for data and other resources. The user portal provides access to the Cloud computing environment for both users and system administrators. Service level management provides Cloud computing resource allocation and management such that the service levels needed are met. Service Level Agreement (SLA) planning and fulfillment provide pre-arrangement for, and procurement of, Cloud computing resources for which a future requirement is anticipated in accordance with an SLA.

Workloads layer 5208 provides functionality for which the cloud computing environment is utilized. Examples of workloads and functions which may be provided from this layer include mapping and navigation; software development and lifecycle management; virtual classroom education delivery; data analytics processing; transaction processing; and resource credit management. As mentioned above, all of the foregoing examples described with respect to FIG. 52 are illustrative, and the disclosure is not limited to these examples.

### LISTING OF DRAWING ELEMENTS

100 user agency and capability augmentation system
102 user
104 wearable computing and biosignal sensing device
106 biosignals
108 background material
110 sensor data
112 other device data
114 application context
116 prompt composer
118 GenAI
120 multimodal output stage
122 output modalities
124 utterance
126 written text
128 multimodal artifact
130 other user agency
132 encoder/parser
134 non-language user agency device
136 biosignals prompt
138 context prompt
140 user input prompt
142 shared data
144 prompt
146 multimodal output
148 output mode selection signal
200 biosignals subsystem
202 additional biosensors
204 biosignals classifier
206 EEG tokenizer
208 kinematic tokenizer
210 additional tokenizers
300 context subsystem
302 raw background material tokenizer
304 final background material tokenizer
306 raw sensor data tokenizer
308 final sensor data tokenizer
310 raw device data tokenizer
312 final device data tokenizer
314 raw application context tokenizer
316 final application context tokenizer
318 context prompt composer
320 preliminary tokens
322 final tokens
400 routine
402 block
404 block
406 block
408 block
410 block
412 block
414 block
416 decision block
418 block
420 block
422 block
500 turn-taking capability augmentation system
502 conversation partner
504 speech
506 EEG or similar biosignals
508 conversation history and user knowledge of a topic
510 camera data
512 microphone data
600 user agency and capability augmentation system with output adequacy feedback
602 decision block
604 decision block
606 Operation may proceed
608 original prompt
610 unexpected output machine learning model
612 new prompt
614 negative rejection feedback tokens
700 routine
702 simultaneous user
704 network
800 exemplary tokenizer
802 input data
804 tokenizable element
806 token
808 tokenized output
900 BCI headset system
902 augmented reality display lens
904 top cover
906 adjustable strap
908 padding
910 ground/reference electrode
912 ground/reference electrode adjustment dial
914 biosensor electrodes
916 battery cell
918 fit adjustment dial
920 control panel cover
922 control panel
924 biosensor electrode adjustment dials
926 auxiliary electrode ports
928 power switch
930 USB port
932 forward housing
934 smart phone
936 smart phone slot
1000 routine
1002 block
1004 block
1006 block
1008 block
1010 block
1012 block
1014 block
1016 block
1018 block
1020 decision block
1022 block
1024 block
1100 routine
1102 block
1104 block
1106 block
1108 block
1110 block
1112 block
1114 block
1116 block
1200 routine
1202 block
1204 block
1206 block
1208 block
1210 block
1212 block
1214 block
1216 block
1300 routine
1302 block
1304 block
1306 block
1308 block
1310 block
1312 block
1314 block
1316 block
1318 block
1400 routine
1402 block
1404 block
1406 block
1408 block
1410 block
1412 block
1500 routine
1502 block
1504 block
1506 block
1508 block
1510 block
1512 block
1514 block
1600 routine
1602 block
1604 block
1606 block
1608 block
1610 block
1612 block
1614 block
1616 block
1700 routine
1702 block
1704 block
1706 block
1708 block
1710 block
1712 block
1714 block
1716 block
1800 routine
1802 block
1804 block
1806 block
1808 block
1810 block
1812 block
1814 block
1816 block
1900 routine
1902 block
1904 block
1906 block
1908 block
1910 block
1912 block
1914 block
1916 block
2000 routine
2002 block
2004 block
2006 block
2008 block
2010 block
2012 block
2014 block
2100 routine
2102 block
2104 block
2106 block
2108 block
2110 block
2112 block
2114 block
2116 block
2200 routine
2202 block
2204 block
2206 block
2208 block
2210 block
2212 block
2214 block
2216 block
2300 routine
2302 block
2304 block
2306 block
2308 block
2310 block
2312 block
2314 block
2316 block
2400 routine
2402 block
2404 block
2406 block
2408 block
2410 block
2412 block
2414 block
2416 block
2500 routine
2502 block
2504 block
2506 block
2508 block
2510 block
2512 block
2514 block
2516 block
2600 routine
2602 block
2604 block
2606 block
2608 block
2610 block
2612 block
2614 block
2616 block
2700 routine
2702 block
2704 block
2706 block
2708 block
2710 block
2712 block
2714 block
2716 block
2800 routine
2802 block
2804 block
2806 block
2808 block
2810 block
2812 block
2814 block
2816 block
2900 routine
2902 block
2904 block
2906 block
2908 block
2910 block
2912 block
3000 routine
3002 block
3004 block
3006 block
3008 block
3010 block
3012 block
3100 routine
3102 block
3104 block
3106 block
3108 block
3110 block
3112 block
3114 block
3116 block
3200 routine
3202 block
3204 block
3206 block
3208 block
3210 block
3300 routine
3302 block
3304 block
3306 block
3308 block
3310 block
3312 block
3314 block
3316 block
3400 routine
3402 block
3404 block
3406 block
3408 block
3410 block
3412 block
3500 routine
3502 block
3504 block
3506 block
3508 block
3510 block
3512 block
3514 block
3516 block
3600 routine
3602 block
3604 block
3606 block
3608 block
3610 block
3612 block
3614 block
3616 block
3700 BCI+AR environment
3702 human user
3704 sensor
3706 EEG analog to digital converter
3708 Audio/Video/Haptic Output
3710 processing
3712 augmented reality glasses
3714 strap
3716 BCI
3800 augmented reality device logic
3802 processing units
3804 CPU
3806 GPU
3808 NPU
3810 WiFi
3812 Bluetooth
3814 speakers
3816 microphones
3818 memory
3820 logic
3822 graphics engine
3824 camera
3826 visual display
3828 vibration/haptic driver
3830 cloud
3832 Bluetooth keyboard
3900 block diagram of nonverbal multi-input and feedback device
3902 application
3904 multimodal input and intent detection
3906 context awareness
3908 GenAI
3910 output and action
3912 touch
3914 bio-signals
3916 keyboard
3918 facial tracking
3920 eye and pupil tracking
3922 alternative inputs
3924 biometrics
3926 environment
3928 object recognition
3930 facial recognition
3932 voice recognition
3934 date and time
3936 history
3938 location
3940 proximity
3942 other metadata
3944 flat screen display
3946 augmented/virtual reality
3948 virtual AI assistant
3950 synthesized voice
3952 prosthetic device
3954 social media and messaging
3956 media consumption
4000 block diagram of a single framework of a nonverbal multi-input and feedback
device
4002a sensor
4002b sensor
4002c sensor
4002d sensor
4002e sensor
4002f sensor
4004 input gesture
4006 context awareness
4008 machine learning
4010 output expression
4012 destination
4014 touch
4016 movement
4018 mental
4020 glances
4022 audible
4024 breath
4026 time synchronization
4028 configure data sources
4030 configure data processing parameters
4032 configure timing
4034 metadata tagging
4036 acquire analog data streams
4038 convert to digital data streams
4040 analyze data streams
4042 execute digital operations for actuation
4044 text
4046 symbol
4048 color
4050 image
4052 sound
4054 vibration
4056 mobile
4058 wearable 1
4060 wearable 2
4062 implant 1
4064 implant 2
4066 prosthetic 1
4100 block diagram of nonverbal multi-input and feedback device
4102 analog input
4104 sensors
4106 processing
4108 digital output
4110 output methods
4112 historical data
4114 learning system
4116 network
4118 personalization settings
4120 metadata
4122 application
4124 primary and secondary languages
4126 visual
4128 audible
4130 written
4200 logical diagram of a user wearing an augmented reality headset
4202 head motion analog input
4204 headset with head motion detection sensors
4206 user selects options through head motion
4208 application creates output based on the user's selected options
4210 condition that system detects the user is away from home
4212 send output to a caregiver via text message
4300 logical diagram of a user wearing an augmented reality headset
4302 user wears an EEG-based brain-computer interface headset
4304 electrodes that are contacting the scalp
4306 streaming analog data may be acquired from the brainwave activity
4308 set of words to choose from
4310 phrase they've composed
4400 diagram of a use case including a user wearing an augmented reality headset
4402 augmented reality headset combined with a brain computer interface
4404 head motion analog input and brainwave activity
4406 set of words to choose from
4408 phrase they've composed
4500 flow diagram
4502 human user
4504 electrode sensors
4506 brain computer interface headset and firmware
4508 augmented reality mobile application
4510 machine learning capture and training
4512 augmented reality headset
4514 capture
4516 analog-to-digital
4518 intent detection
4520 action trigger
4522 user interface
4524 machine learning parameters
4526 raw data capture
4528 stimulus placement and timing
4530 augmented reality environment
4532 training data
4534 training and data analysis
4536 stimulus placement and timing
4538 evoke potential stimulus
4540 output and action
4600 flow diagram
4602 multimodal, multi-sensory system for communication and control
4604 wireless neck and head tracking
4606 wireless brain tracking
4608 central sensors
4610 peripheral sensors
4612 analog to digital signal processor
4614 analog to digital signal processor
4616 analog to digital subsystem
4618 sensor service subsystem
4620 collector subsystem
4622 real-time clock
4624 recognizer
4626 classifier
4628 wireless streamer
4630 serial streamer
4632 traditional workstation
4634 cloud
4636 miniaturized mobile computing system
4638 wireless muscle tracking
4640 wireless eye pupil tracking
4642 controller subsystem
4644 command queue
4646 command
4648 serial read
4650 BT write callback
4652 peripherals subsystem
4654 audio feedback
4656 haptic feedback
4658 OLED visual feedback
4660 storage subsystem
4700 block diagram
4702 data authentication
4704 sensory device and mobile system
4706 cloud system
4708 companion application
4710 firmware
4712 data analysis
4714 SQS message queuing
4716 server computing
4718 elastic computing
4720 object storage
4722 database
4800 block diagram
4802 integrated virtual AI assistant and web services
4804 audio input processor
4806 AI communication library
4808 virtual assistant
4810 AI directive sequencer library
4812 capability agent
4814 active focus manager library
4816 gesture
4818 sensor
4820 application user interface
4822 actuation subsystem
4824 visual feedback
4826 audible feedback
4828 haptic feedback
4900 block diagram
4902 AI virtual assistant
4904 content management system
4906 cloud data logs
4908 authentication
4910 speech generation
4912 runtime environment
4914 serverless cloud
4916 API gateway
4918 application
4920 TTS voice engine
4922 email client
4924 account analytics
4926 marketing analytics
4928 application analytics
4930 vocabulary
4932 user events
4934 customer relations management
4936 app store
5000 computing device
5002 system memory
5004 processing units
5006 Random access memory (RAM)
5008 I/O interfaces
5010 cache memory
5012 external devices
5014 display
5016 network adapter
5018 storage system
5020 program/utility
5022 program modules
5024 bus
5026 sensors
5028 actuators
5100 cloud computing system
5102 computing device
5104 BCI
5106 cloud servers
5108 wearable computing and biosignal sensing device
5200 cloud computing functional abstraction layers
5202 hardware and software layer
5204 virtualization layer
5206 management layer
5208 workloads layer

Various functional operations described herein may be implemented in logic that is referred to using a noun or noun phrase reflecting said operation or function. For example, an association operation may be carried out by an “associator” or “correlator”. Likewise, switching may be carried out by a “switch”, selection by a “selector”, and so on.

Within this disclosure, different entities (which may variously be referred to as “units,” “circuits,” other components, etc.) may be described or claimed as “configured” to perform one or more tasks or operations. This formulation-[entity] configured to [perform one or more tasks]—is used herein to refer to structure (i.e., something physical, such as an electronic circuit). More specifically, this formulation is used to indicate that this structure is arranged to perform the one or more tasks during operation. A structure may be said to be “configured to” perform some task even if the structure is not currently being operated. A “credit distribution circuit configured to distribute credits to a plurality of processor cores” is intended to cover, for example, an integrated circuit that has Circuitry that performs this function during operation, even if the integrated circuit in question is not currently being used (e.g., a power supply is not connected to it). Thus, an entity described or recited as “configured to” perform some task refers to something physical, such as a device, circuit, memory storing program instructions executable to implement the task, etc. This phrase is not used herein to refer to something intangible.

The term “configured to” is not intended to mean “configurable to.” An unprogrammed field programmable gate array (FPGA), for example, would not be considered to be “configured to” perform some specific function, although it may be “configurable to” perform that function after programming.

Reciting in the appended claims that a structure is “configured to” perform one or more tasks is expressly intended not to invoke 35 U.S.C. § 112 (f) for that claim element. Accordingly, claims in this application that do not otherwise include the “means for” [performing a function] construct should not be interpreted under 35 U.S.C § 112 (f).

As used herein, the term “based on” is used to describe one or more factors that affect a determination. This term does not foreclose the possibility that additional factors may affect the determination. That is, a determination may be solely based on specified factors or based on the specified factors as well as other, unspecified factors. Consider the phrase “determine A based on B.” This phrase specifies that B is a factor that is used to determine A or that affects the determination of A. This phrase does not foreclose that the determination of A may also be based on some other factor, such as C. This phrase is also intended to cover an embodiment in which A is determined based solely on B. As used herein, the phrase “based on” is synonymous with the phrase “based at least in part on.”

As used herein, the phrase “in response to” describes one or more factors that trigger an effect. This phrase does not foreclose the possibility that additional factors may affect or otherwise trigger the effect. That is, an effect may be solely in response to those factors or may be in response to the specified factors as well as other, unspecified factors. Consider the phrase “perform A in response to B.” This phrase specifies that B is a factor that triggers the performance of A. This phrase does not foreclose that performing A may also be in response to some other factor, such as C. This phrase is also intended to cover an embodiment in which A is performed solely in response to B.

As used herein, the terms “first,” “second,” etc. are used as labels for nouns that they precede and do not imply any type of ordering (e.g., spatial, temporal, logical, etc.), unless stated otherwise. For example, in a register file having eight registers, the terms “first register” and “second register” may be used to refer to any two of the eight registers, and not, for example, just logical registers 0 and 1.

When used in the claims, the term “or” is used as an inclusive or and not as an exclusive or. For example, the phrase “at least one of x, y, or z” means any one of x, y, and z, as well as any combination thereof.

Having thus described illustrative embodiments in detail, it will be apparent that modifications and variations are possible without departing from the scope of the disclosure as claimed. The scope of disclosed subject matter is not limited to the depicted embodiments but is rather set forth in the following Claims.

Terms used herein may be accorded their ordinary meaning in the relevant arts, or the meaning indicated by their use in context, but if an express definition is provided, that meaning controls.

Herein, references to “one embodiment” or “an embodiment” do not necessarily refer to the same embodiment, although they may. Unless the context clearly requires otherwise, throughout the description and the claims, the words “comprise,” “comprising.” and the like are to be construed in an inclusive sense as opposed to an exclusive or exhaustive sense; that is to say, in the sense of “including, but not limited to.” Words using the singular or plural number also include the plural or singular number respectively, unless expressly limited to a single one or multiple ones. Additionally, the words “herein,” “above,” “below” and words of similar import, when used in this application, refer to this application as a whole and not to any particular portions of this application. When the claims use the word “or” in reference to a list of two or more items, that word covers all of the following interpretations of the word: any of the items in the list, all of the items in the list and any combination of the items in the list, unless expressly limited to one or the other. Any terms not expressly defined herein have their conventional meaning as commonly understood by those having skill in the relevant art(s).

It is to be understood that the disclosed subject matter is not limited in its application to the details of construction and to the arrangements of the components set forth in the following description or illustrated in the drawings. The disclosed subject matter is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, systems, methods and media for carrying out the several purposes of the disclosed subject matter. It is important, therefore, that the claims be regarded as including such equivalent constructions insofar as they do not depart from the spirit and scope of the disclosed subject matter.

What is claimed is:

1. The system comprising:
a context subsystem configured to receive at least one of background material, sensor data, and other device data as information that is used in part to infer a context for a user;
a biosignals subsystem configured to receive at least one of a physically sensed signal and a neurologically sensed signal from the user;
a prompt composer configured to receive an input from at least one of the context subsystem and the biosignals subsystem to generate a prompt that identifies at least one of a requested output modality and a desired output modality;
a pre-trained Generative Artificial Intelligence (GenAI) model configured to utilize the prompt to generate a multimodal output;
an output stage configured to transform the multimodal output into at least one form of user agency, user capability augmentation, and combinations thereof; and
logic to:
tokenize the at least one of the background material, the sensor data, and the other device data into context tokens suitable to prompt the GenAI model;
tokenize the at least one of the physically sensed signal and the neurologically sensed signal into biosignal tokens suitable to prompt the GenAI model;
generate a context prompt from at least one of the context tokens and the biosignal tokens;
prompt the GenAI model with the context prompt and receive the multimodal output from the GenAI model; and
transform the multimodal output into the at least one form of the user agency, the user capability augmentation, and combinations thereof.

2. The system of claim 1, wherein the context subsystem receives the at least one of the sensor data and the other device data from at least one of a camera and a microphone array.

3. The system of claim 1, wherein the at least one form of the user agency includes neural stimulation to the user with Transcranial Direct Current Stimulation (tDCS).

4. The system of claim 1, wherein the biosignals subsystem receives data from biometric sensors for at least one of electroencephalography (EEG), electrocorticography (ECoG), electrocardiogram (ECG or EKG), electromyography (EMG), electrooculography (EOG), pulse determination, heart rate variability determination, blood sugar sensing, and dermal conductivity determination.

5. The system of claim 1, wherein the prompt composer constructs at least one of:
a single token;
a string of tokens;
a series of conditional or unconditional commands suitable to prompt the GenAI model;
tokens that identify at least one of the requested output modality and the desired output modality;
an embedding to be provided separately to the GenAI model for use in an intermediate layer of the GenAI model; and
multiple tokenized sequences at once that constitute a series of conditional commands.

6. The system of claim 1, wherein the pre-trained GenAI model is at least one of large language models (LLMs), Generative Pre-trained Transformer (GPT) models, text-to-image creators, visual art creators, and generalist agent models.

7. The system of claim 1, wherein the output stage is configured to receive an output mode selection signal from the user through biosignals, wherein the output mode selection signal at least one of:
instructs the output stage of a choice between the multimodal outputs; and
instructs the output stage to direct one or more of alternative multimodal outputs to alternate endpoints.

8. The system of claim 1, wherein the multimodal output is in the form of at least one of text-to-speech utterances, written text, multimodal artifacts, other user agency supportive outputs, and commands to a non-language user agency device.

9. The system of claim 1, the output stage including an output adequacy feedback system, including logic to:
detect an event related potential (ERP) in response to a multimodal output suggestion;
on condition the ERP is detected, performing at least one of:
provide feedback to at least one of:
the user; and
the prompt composer, wherein the prompt composer provides the feedback to the GenAI model;
wherein the feedback includes at least one of the ERP and a current context state;
record the ERP to the multimodal output suggestion;
automatically reject the multimodal output suggestion, generate new prompts with rejection feedback tokens, and send the rejection feedback tokens to the prompt composer; and
on condition no ERP is detected:
allow the multimodal output suggestion to proceed.

10. The system of claim 1, further comprising:
an encoder/parser framework for additionally encoding multimodal output; and
logic to:
provide control commands to control at least one of:
a non-language user agency device;
a robot system; and
smart AI-powered devices.

11. A method comprising:
receiving, by a context subsystem, at least one of background material, sensor data, and other device data as information that is used in part to infer a context for a user;
receiving, by a biosignals subsystem, at least one of a physically sensed signal and a neurologically sensed signal from the user;
receiving, by a prompt composer, an input from at least one of the context subsystem and the biosignals subsystem;
generating, by the prompt composer, a prompt that identifies at least one of a requested output modality and a desired output modality;
utilizing, by a pre-trained Generative Artificial Intelligence (GenAI) model, the prompt to generate a multimodal output;
transforming, by an output stage, the multimodal output into at least one form of user agency, user capability augmentation, and combinations thereof.

12. The method of claim 11, wherein receiving, by the context subsystem, includes receiving the at least one of the sensor data and the other device data from at least one of a camera and a microphone array.

13. The method of claim 11, wherein the at least one form of the user agency includes neural stimulation to the user with Transcranial Direct Current Stimulation (tDCS).

14. The method of claim 11, wherein receiving, by the biosignals subsystem, includes receiving data from biometric sensors for at least one of electroencephalography (EEG), electrocorticography (ECoG), electrocardiogram (ECG or EKG), electromyography (EMG), electrooculography (EOG), pulse determination, heart rate variability determination, blood sugar sensing, and dermal conductivity determination.

15. The method of claim 11, wherein generating, by the prompt composer, includes generating at least one of:
a single token;
a string of tokens;
a series of conditional or unconditional commands suitable to prompt the GenAI model;
tokens that identify at least one of the requested output modality and the desired output modality;
an embedding to be provided separately to the GenAI model for use in an intermediate layer of the GenAI model; and
multiple tokenized sequences at once that constitute a series of conditional commands.

16. The method of claim 11, wherein the pre-trained GenAI model is at least one of large language models (LLMs), Generative Pre-trained Transformer (GPT) models, text-to-image creators, visual art creators, and generalist agent models.

17. The method of claim 11, further comprising: receiving, by the output stage, an output mode selection signal from the user through biosignals, wherein the output mode selection signal at least one of:
instructs the output stage of a choice between the multimodal outputs; and
instructs the output stage to direct one or more of alternative multimodal outputs to alternate endpoints.

18. The method of claim 11, wherein the multimodal output is in the form of at least one of text-to-speech utterances, written text, multimodal artifacts, other user agency supportive outputs, and commands to a non-language user agency device.

19. The method of claim 11, the output stage further including an output adequacy feedback system, the method further comprising:
detecting, using the output adequacy feedback system, an event related potential (ERP) in response to a multimodal output suggestion;
on condition the ERP is detected, performing at least one of:
providing feedback to at least one of:
the user; and
the prompt composer, wherein the prompt composer provides the feedback to the GenAI model;
wherein the feedback includes at least one of the ERP and a current context state;
recording the ERP to the multimodal output suggestion;
automatically rejecting the multimodal output suggestion, generating new prompts with rejection feedback tokens, and sending the rejection feedback tokens to the prompt composer; and
on condition no ERP is detected:
allowing the multimodal output suggestion to proceed.

20. The method of claim 11, further comprising an encoder/parser framework, the method further comprising:
encoding the multimodal output using the encoder/parser framework to provide control commands to control at least one of:
a non-language user agency device;
a robot system; and
smart AI-powered devices.
